(19) United States
(12) Patent Application Publication
(10) Pub. No.: US 2010/0173835 A1
(43) Pub. Date: Jul. 8, 2010

(54) Peptides for Treatment of Obesity

(75) Inventors: Jesper Lau, Farum (DK); Ulrich Sensfuss, København V (DK); Kilian Waldemar Conde-Frieboes, Måløv (DK); Birgitte Schjellerup Wulff, Virum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 12/626,188

(22) Filed: Nov. 25, 2009

Related U.S. Application Data
(60) Provisional application No. 61/117,999, filed on Nov. 26, 2008.

(30) Foreign Application Priority Data
Nov. 25, 2008 (EP) 08169871.4

(51) Int. Cl. A61K 38/12; C07K 7/64; A61P 7/12; A61P 3/04; A61P 35/04; A61P 15/00

(52) U.S. Cl. 514/11; 530/317

(57) ABSTRACT

The present invention relates to novel peptide compounds which are effective in modulating one or more melanocortin receptor types, to the use of the compounds in therapy, to methods of treatment comprising administration of the compounds to patients in need thereof, and to the use of the compounds in the manufacture of medicaments. The compounds of the invention are of particular interest in relation to the treatment of obesity as well as a variety of diseases or conditions associated with obesity.

### FIELD OF THE INVENTION

The present invention relates to novel peptides which are specific to one or more melanocortin receptors with improved water solubility, to the use of said peptides in therapy, to methods of treatment comprising administration of said peptides to patients, and to the use of said peptides in the manufacture of medicaments.

### BACKGROUND OF THE INVENTION

Obesity is a well known risk factor for the development of common diseases such as atherosclerosis, hypertension, type 2 diabetes, dyslipidaemia, coronary heart disease, gallbladder disease, osteoarthritis, premature death, certain types of cancer and various other malignancies. It also causes considerable problems through reduced motility and decreased quality of life. In the industrialized western world the prevalence of obesity has increased significantly in the past few decades. Only a few pharmacological treatments are available to date, namely Sibutramine (Abbot, acting via serotonergic and noradrenaline mechanisms), Orlistat (Roche, reducing fat uptake from the gut). Because obesity represents a very high risk factor in serious and even fatal common diseases, its treatment should be a high public health priority and there is a need for pharmaceutical compounds useful in the treatment of obesity.

Pro-opiomelanocortin (POMC) is the precursor of the melanocortin family of peptides, which include α-, β- and γ-melanocyte stimulating hormone (MSH) peptides and adrenocorticotropic hormone (ACTH), as well as other peptides such as 3-endorphin. POMC is expressed in neurons of the central and peripheral nervous system and in the pituitary. Several of the melanocortin peptides, including ACTH and α-MSH, have been shown to have appetite-suppressing activity when administered to rats by intracerebroventricular (icy) injection [Vergoni et al, European Journal of Pharmacology 179, 347-355 (1990)]. An appetite-suppressing effect is also obtained with the artificial cyclic α-MSH analogue, MT-II.

Five melanocortin receptor subtypes, MC1-5 receptors have been identified. MC1, MC2 and MC5 receptors are mainly expressed in peripheral tissues, whereas MC3 and MC4 receptors are mainly centrally expressed. MC3 receptors are also expressed in several peripheral tissues. In addition to being involved in energy homeostasis, MC3 receptors have also been suggested to be involved in several inflammatory diseases. It has been suggested that MC5 receptors are involved in exocrine secretion and in inflammation. MC4 receptors have been shown to be involved in the regulation of body weight and feeding behavior, as MC4 knock-out mice develop obesity [Huzar et al., Cell 88, 131-141 (1997)] and common variants near MC4 receptor have been found to be associated with fat mass, weight and risk of obesity [Loos et al. Nat. Genet., 40(6):768-75 (2008)]. Furthermore, studies with mice showed that overexpression in the mouse brain of the melanocortin receptor antagonists agouti protein and agouti-related protein (AGRP), led to the development of obesity [Kleibig et al., PNAS 92, 4728-4732 (1995)]. Moreover, icy injection of a C-terminal fragment of AGRP increases feeding and antagonizes the inhibitory effect of α-MSH on food intake.

MC4 receptor agonists could serve as anorectic drugs and/or energy expenditure increasing drugs and be useful in the treatment of obesity or obesity-related diseases, as well as in the treatment of other diseases, disorders or conditions which may be ameliorated by activation of MC4 receptor. On the other hand, MC4 receptor antagonists may be useful in the treatment of cachexia or anorexia, of waisting in frail elderly patients, chronic pain, neuropathy and neurogenic inflammation.

A large number of patent applications disclose various classes of non-peptidic small molecules as melanocortin receptor modulators, of which examples hereof are WO 03/009850, WO 03/007949 and WO 02/081443. The use of peptides as melanocortin receptor modulators is disclosed in a number of patent documents, e.g. WO 03/006620, U.S. Pat. No. 5,731,408 and WO 98/27113. Hadley [Pigment Cell Res. (1991) 4:180-185] reported a prolonged effect of specific melanotropic peptides conjugated to fatty acids, the prolongation effected by a transformation of the modulators from being reversibly acting to being irreversibly acting being caused by the conjugated fatty acids.

### SUMMARY OF THE INVENTION

The present invention relates to novel peptides which are specific to one or more melanocortin receptors with improved water solubility at neutral pH, to the use of said peptides in therapy, to methods of treatment comprising administration of said peptides to patients, and to the use of said peptides in the manufacture of medicaments.

The present inventors have surprisingly found that specific peptide conjugates have a high modulating effect on one or more melanocortin receptors, i.e., the MC1, MC2, MC3, MC4 or MC5. Accordingly, in a first embodiment (embodiment 1), the invention relates to compounds (more particularly compounds acting as melanocortin receptor agonists or antagonists) of formula I:

R¹—R²—C(═O)—R³—S¹—Z¹—Z²—Z³—Z⁴—Z⁵—Z⁶-c[X¹—X²—X³—-Arg-X⁴—X⁵]—Z⁷—R⁴ [I]

wherein
R¹ represents tetrazol-5-yl or carboxy;
R² represents a straight-chain, branched and/or cyclic C₆₋₂₀alkylene, C₆₋₂₀alkenylene or C₆₋₂₀alkynylene which may optionally be substituted with one or more substituents selected from halogen, hydroxy and aryl;
R³ is absent or represents —NH—S(═O)₂—(CH₂)₃₋₅—C(═O)— or a peptide fragment comprising one or two amino acid residues derived from natural or unnatural amino acids and containing at least one carboxy group;
wherein the side chains of R³ must not contain amino, guanidino, imidazolyl or other basic groups positively charged at neutral pH;
S¹ is absent or represents a glycolether-based structure according to one of the formulas IIa-IIh;

—HN—CH₂—CH₂—O—CH₂—CH₂—O—CH₂—C(═O)— [IIa]

—[HN—CH₂—CH₂—O—CH₂—CH₂—O—CH₂—C(═O)]₂— [IIb]

—[HN—CH₂—CH₂—O—CH₂—CH₂—O—CH₂—C(═O)]₃₋₅— [IIc]

—[HN—CH₂—CH₂—O—CH₂—CH₂—O—CH₂—CH₂—NH—C(═O)—CH₂—CH₂—CH₂—C(═O)]₁₋₃— [IId]

—[HN—CH₂—CH₂—O—CH₂—CH₂—O—CH₂—CH₂—NH—C(═O)—CH₂—O—CH₂—C(═O)]₁₋₃— [IIe]

—[HN—CH₂—CH₂—O—CH₂—CH₂—O—CH₂—CH₂—O—CH₂—CH₂—O—CH₂—CH₂—C(═O)]₁₋₃— [IIf]

—HN—CH₂—CH₂—[O—CH₂—CH₂]₂₋₁₂—O—CH₂—C(═O)— [IIg]

—HN—CH₂—CH₂—[O—CH₂—CH₂]₄₋₁₂—O—CH₂—CH₂—C(═O)— [IIg]

Z¹ is absent or represents a peptide fragment comprising one to four amino acid residues derived from natural or unnatural amino acids;
wherein the side chains of Z¹ must not contain amino, guanidino, imidazolyl or other basic groups positively charged at neutral pH;
Z² represents Gly, β-Ala, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;
Z³ represents Gly, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;
Z⁴ represents Gly, Ala, β-Ala, D-Ala, Pro, D-Pro, Hyp, D-Hyp, Ser, D-Ser, homoSer, D-homoSer, Thr, D-Thr, Tyr, D-Tyr, Phe, D-Phe, Gln, D-Gln, Asn, D-Asn, 2-PyAla, D-2-PyAla, 3-PyAla, D-3-PyAla, 4-PyAla, D-4-PyAla, His or D-His;
with the proviso that not more than one of residues Z², Z³ and Z⁴ is His or D-His;
Z⁵ represents a structure according to one of the formulas IIIa, IVa, Va, VIa, VIIa, VIIIa, IXa, Xa, IIIb, IVb, Vb, VIb, VIIb, VIIIb, IXb, or Xb;

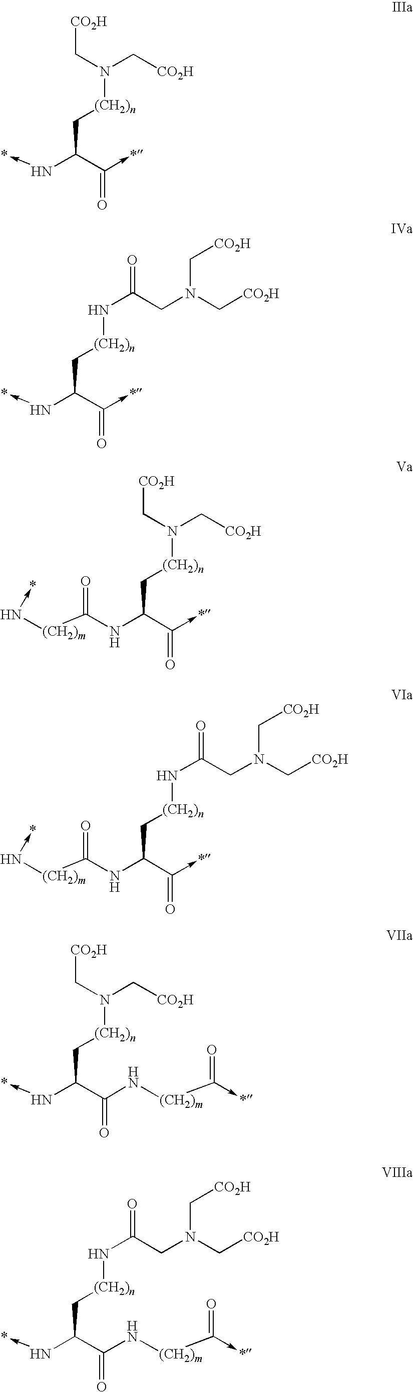

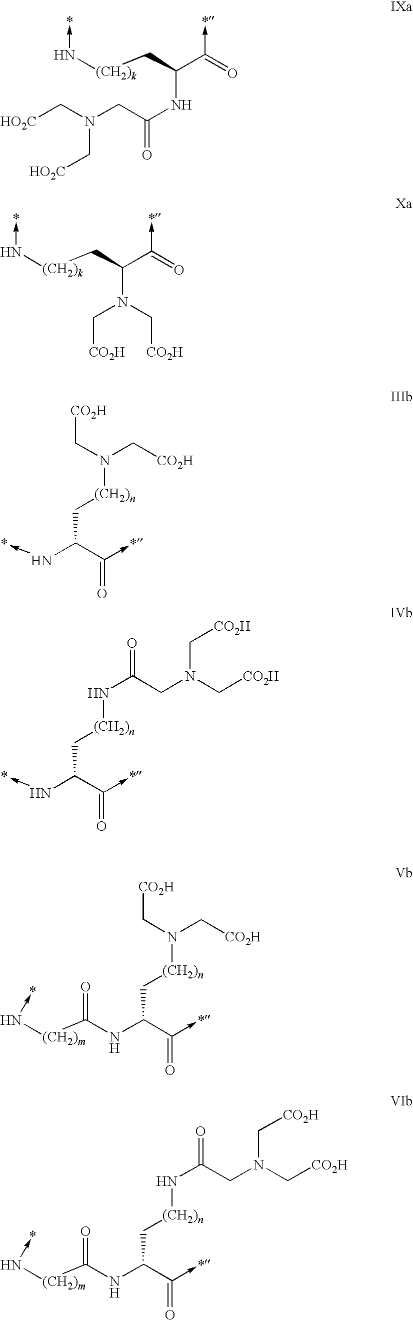

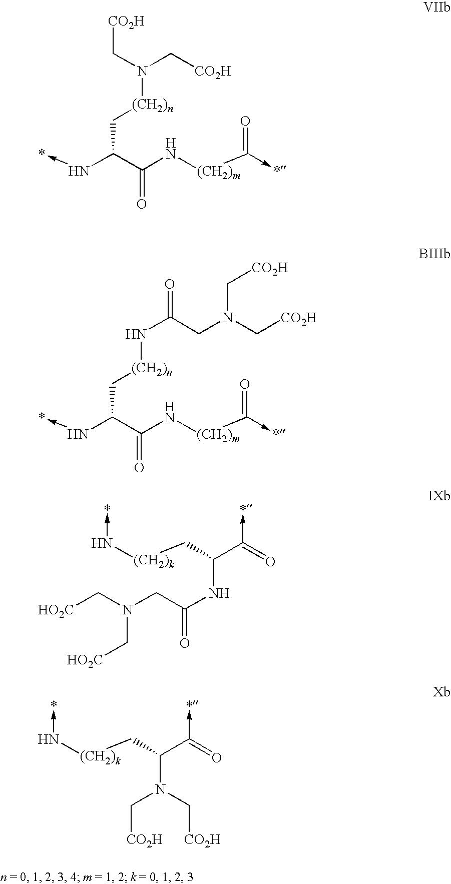

wherein n in formulas IIIa to VIIIa and IIIb to VIIIb is 0, 1, 2, 3 or 4,
m in formulas Va to VIIIa and Vb to VIIIb is 1 or 2,
k in formulas IXa, Xa, IXb and Xb is 0, 1, 2 or 3;
Z⁶ in formula I represents Ala, D-Ala, Val, D-Val, Leu, D-Leu, Ile, D-Ile, Met, D-Met, Nle, D-Nle, Phe, D-Phe, Tyr, D-Tyr, Trp or D-Trp;
X¹ represents Glu, Asp, Cys, homoCys, Lys, Orn, Dab or Dap;
X² represents His, Cit, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, Aze, Pip, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein one or more hydrogens on the phenyl moiety of said Phe may optionally and independently be substituted by a substituent selected among halogen, hydroxy, alkoxy, nitro, benzoyl, methyl, trifluoromethyl and cyano;
X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;
X⁴ represents Trp, 2-Nal, (3-benzo[b]thienyl)alanine or (S)-2,3,4,9-tetrahydro-1H-β-carboline-3-carboxylic acid;
X⁵ represents Glu, Asp, Cys, homoCys, Lys, Orn, Dab or Dap;
wherein X¹ and X⁵ are joined, rendering the compound of formula I cyclic, either via a disulfide bridge deriving from X¹ and X⁵ both independently being Cys or homoCys, or via an amide bond formed between a carboxylic acid in the side-chain of X¹ and an amino group in the side-chain of X⁵, or between a carboxylic acid in the side-chain of X⁵ and an amino group in the side-chain of X¹;
Z⁷ is absent or represents a peptide fragment comprising one to three amino acid residues derived from natural or unnatural amino acids;
wherein the side chains of Z⁷ must not contain amino, guanidino, imidazolyl or other basic groups positively charged at neutral pH;
R⁴ represents OR′ or N(R′)₂, wherein each R′ independently represents hydrogen or represents C₁₋₆alkyl, C₂₋₆alkenyl or C₂₋₆alkynyl which may optionally be substituted with one or more hydroxy;
and pharmaceutically acceptable salts, prodrugs and solvates thereof.

The invention further relates to the use of compounds of the invention in therapy, to pharmaceutical compositions comprising compounds of the invention, and to the use of compounds of the invention in the manufacture of medicaments.

### DESCRIPTION OF THE INVENTION

Among further embodiments of compounds of the present invention are the following:
2. A compound according to embodiment 1, wherein
   R² represents straight-chain α,ω-C₁₂₋₂₀alkylene, α,ω-C₁₂₋₂₀alkenylene or α,ω-C₁₂₋₂₀alkynylene which may optionally be substituted with one or more hydroxyl;
   R³ is absent or represents —NH—S(═O)₂—(CH₂)₃—C(═O)—, Glu, D-Glu, γ-Glu or D-γ-Glu;
   Z¹ is absent or represents a peptide fragment comprising one to four amino acid residues selected from Gly, β-Ala, Ser, D-Ser, Thr, D-Thr, Asn, D-Asn, Gln, D-Gln, Glu, D-Glu, Asp, D-Asp, Ala, D-Ala, Pro, D-Pro, Hyp or D-Hyp;
   Z² represents Gly, β-Ala, Ser, D-Ser, Thr, D-Thr, Asn, D-Asn, Gln, D-Gln, Glu, D-Glu, Asp, D-Asp, Ala, D-Ala, Pro, D-Pro, Hyp or D-Hyp;
   Z³ represents Gly, β-Ala, Ser, D-Ser, Thr, D-Thr, Asn, D-Asn, Gln, D-Gln, Glu, D-Glu, Asp, D-Asp, Ala, D-Ala, Pro, D-Pro, Hyp or D-Hyp;
   Z⁴ represents Gly, Ala, Ser, homoSer, Thr, Tyr, Phe, Gln, Asn, 2-PyAla, 3-PyAla, 4-PyAla or His;
   Z⁵ represents a structure according to one of the formulas IIIa, IVa, Va, VIa, VIIa, VIIIa, IXa or Xa;
   Z⁶ represents Ala, Val, Leu, Ile, Met or Nle;
   X¹ represents Glu or Asp;
   X² represents Hyp, Pro, Aze or Pip;
   X³ represents D-Phe;
   X⁴ represents Trp;
   X⁵ represents Lys or Orn;
   Z⁷ is absent;
   and R⁴ represents OR′ or N(R′)₂, wherein each R¹ independently represents hydrogen or C₁₋₃alkyl.
3. A compound according to any of embodiments 1-2, wherein R¹—R² represents 13-(tetrazol-5-yl)tridecyl, 14-(tetrazol-5-yl)tetradecyl, 15-(tetrazol-5-yl)pentadecyl, 16-(tetrazol-5-yl)hexadecyl, 17-(tetrazol-5-yl)heptadecyl or 18-(tetrazol-5-yl)octadecyl.
4. A compound according to any of embodiments 1-2, wherein R¹—R² represents 15-(tetrazol-5-yl)pentadecyl.
5. A compound according to any of embodiments 1-2, wherein R¹—R² represents 16-(tetrazol-5-yl)hexadecyl.
6. A compound according to any of embodiments 1-2, wherein R¹—R² represents 13-carboxytridecyl, 14-carboxytetradecyl, 15-carboxypentadecyl, 16-carboxyhexadecyl, 17-carboxyheptadecyl, 18-carboxyoctadecyl or 19-carboxynonadecyl.
7. A compound according to any of embodiments 1-2, wherein R¹—R² represents 14-carboxytetradecyl, 16-carboxyhexadecyl or 18-carboxyoctadecyl.
8. A compound according to any of embodiments 1-2, wherein R¹—R² represents 14-carboxytetradecyl.
9. A compound according to any of embodiments 1-2, wherein R¹—R² represents 16-carboxyhexadecyl
10. A compound according to any of embodiments 1-2, wherein R¹—R² represents 18-carboxyoctadecyl.
11. A compound according to any of embodiments 1-10, wherein R³ is absent.
12. A compound according to any of embodiments 1-10, wherein R³ represents —NH—S(═O)₂—(CH₂)₃—C(═O)—.
13. A compound according to any of embodiments 1-10, wherein R³ represents γ-Glu.
14. A compound according to any of embodiments 1-13, wherein S¹ is absent.
15. A compound according to any of embodiments 1-13, wherein S¹ represents a structure according to formulas IIa, IIb or IIc.
16. A compound according to any of embodiments 1-13, wherein S¹ represents a structure according to formula IIa.
17. A compound according to any of embodiments 1-13, wherein S¹ represents a structure according to formula IIb.
18. A compound according to any of embodiments 1-13, wherein S¹ represents a structure according to formula IIc.
19. A compound according to any of embodiments 1-18, wherein Z¹ represents a peptide fragment comprising one to four amino acid residues selected from Gly, β-Ala, Ser, D-Ser, Thr, D-Thr, Asn, D-Asn, Gln, D-Gln, Glu, D-Glu, Asp, D-Asp, Ala, D-Ala, Pro, D-Pro, Hyp or D-Hyp;
20. A compound according to any of embodiments 1-18, wherein Z¹ represents a peptide fragment comprising one to four amino acid residues selected from Gly, Ser, D-Ser, Gln or Glu;
21. A compound according to any of embodiments 1-18, wherein Z¹ represents Gly.
22. A compound according to any of embodiments 1-18, wherein Z¹ represents Glu or Asp.
23. A compound according to any of embodiments 1-18, wherein Z¹ represents Glu.
24. A compound according to any of embodiments 1-18, wherein Z¹ represents Gly-D-Ser-Gln-Ser.
25. A compound according to any of embodiments 1-24, wherein Z² represents Ser, Thr, Gln or Gly.
26. A compound according to any of embodiments 1-24, wherein Z² represents Ser.
27. A compound according to any of embodiments 1-26, wherein Z³ represents Gln, Asn or Ser.
28. A compound according to any of embodiments 1-26, wherein Z³ represents Gln.
29. A compound according to any of embodiments 1-28, wherein Z⁴ represents His, Tyr or Phe.
30. A compound according to any of embodiments 1-28, wherein Z⁴ represents His, Ser or Tyr.
31. A compound according to any of embodiments 1-28, wherein Z⁴ represents His.
32. A compound according to any of embodiments 1-28, wherein Z⁴ represents Ser, Thr, Gln or Asn.
33. A compound according to any of embodiments 1-28, wherein Z⁴ represents Ser.
34. A compound according to any of embodiments 1-28, wherein Z⁴ represents Tyr.
35. A compound according to any of embodiments 1-34, wherein Z⁵ represents Dap(biscarboxymethyl), Dab(biscarboxymethyl), Orn(biscarboxymethyl), Lys(biscarboxymethyl) or homoLys(biscarboxymethyl).
36. A compound according to any of embodiments 1-34, wherein Z⁵ represents Dap(biscarboxymethyl) or Lys(biscarboxymethyl).
37. A compound according to any of embodiments 1-34, wherein Z⁵ represents Dap(BCMA), Dab(BCMA), Orn(BCMA), Lys(BCMA) or homoLys(BCMA).
38. A compound according to any of embodiments 1-34, wherein Z⁵ represents Dap(BCMA), β-Dap(BCMA), Dab(BCMA), Orn(BCMA) or Lys(BCMA).
39. A compound according to any of embodiments 1-34, wherein Z⁵ represents a structure according to one of formulas Va, VIa, VIIa or VIIIa, wherein m is 2.
40. A compound according to any of embodiments 1-34, wherein Z⁵ represents β-Dap(BCMA).
41. A compound according to any of embodiments 1-34, wherein Z⁵ represents Dap(biscarboxymethyl).
42. A compound according to any of embodiments 1-34, wherein Z⁵ represents Lys(biscarboxymethyl).
43. A compound according to any of embodiments 1-34, wherein Z⁵ represents Dap(BCMA).
44. A compound according to any of embodiments 1-34, wherein Z⁵ represents Dab(BCMA).
45. A compound according to any of embodiments 1-34, wherein Z⁵ represents Lys(BCMA).
46. A compound according to any of embodiments 1-34, wherein Z⁵ represents Orn(BCMA).
47. A compound according to any of embodiments 1-34, wherein Z⁵ represents β-Ala-Lys(biscarboxymethyl) or Lys(biscarboxymethyl)-β-Ala.
48. A compound according to any of embodiments 1-34, wherein Z⁵ represents β-Ala-Lys(biscarboxymethyl).
49. A compound according to any of embodiments 1-48, wherein Z⁶ represents Leu, Ile, Nle or Met.
50. A compound according to any of embodiments 1-48, wherein Z⁶ represents Nle.
51. A compound according to any of embodiments 1-50, wherein X¹ is Glu.
52. A compound according to any of embodiments 1-51, wherein X² is Hyp.
53. A compound according to any of embodiments 1-51, wherein X² is Pro.
54. A compound according to any of embodiments 1-52, wherein X⁵ is Lys.
55. A compound according to any of embodiments 1-54, wherein Z⁷ is absent.
56. A compound according to any of embodiments 1-55, wherein R⁴ is NH₂.
57. A compound according to any of embodiments 1-55, wherein R⁴ is OH.
58. A compound according to any of embodiments 1-57, with increased solubility at neutral to weakly basic pH.
59. A compound according to any of embodiments 1-57, with increased solubility at pH from about 6 to about 10.
60. A compound according to any of embodiments 1-57, with increased solubility at pH from about 6 to about 9.
61. A compound according to any of embodiments 1-57, with increased solubility at pH from about 6.5 to about 8.5.
62. A compound according to any of embodiments 1-57, with increased solubility at pH from about 6.5 to about 7.5.
63. A compound according to any of embodiments 1-57, with increased solubility where the pH is about 7.
64. A compound according to embodiment 1, selected from the group consisting of:
   (2-{2-[16-(Tetrazol-5-yl)hexadecanoylamino]ethoxy}ethoxy)acetyl-Gly-Ser-Gln-His-Lys(BCMA)-Nle-c[Glu-Hyp-D-Phe-Arg-Trp-Lys]-NH₂

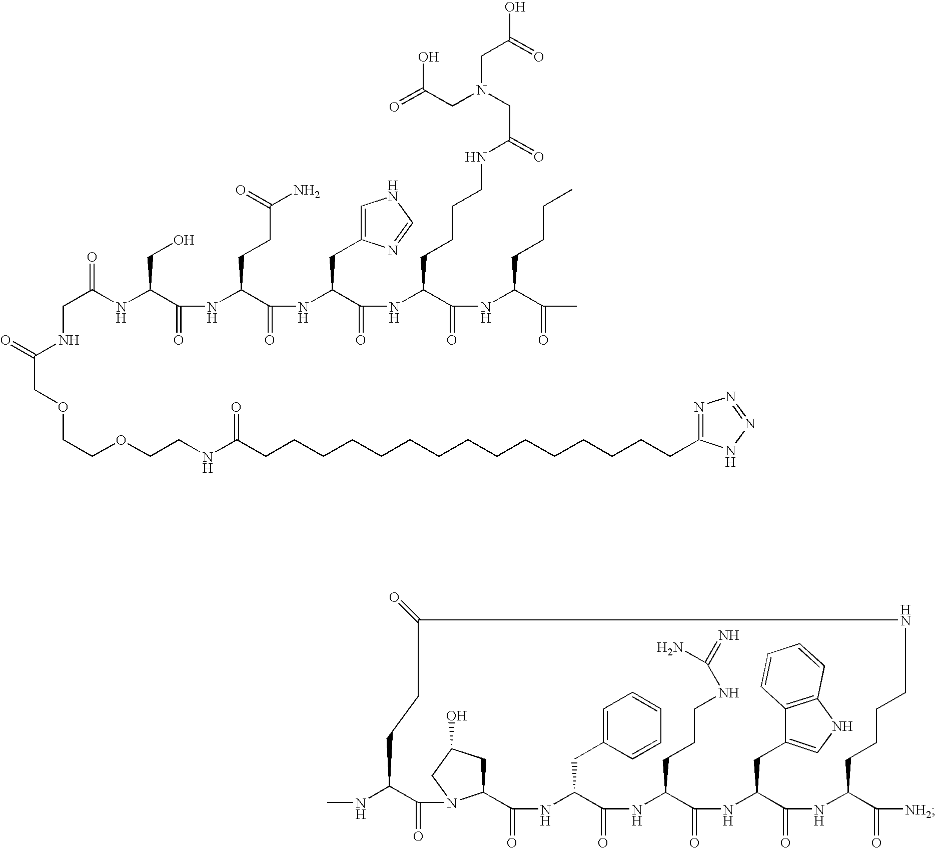

(2-{2-[16-(Tetrazol-5-yl)hexadecanoylamino]ethoxy}ethoxy)acetyl-Gly-Ser-Gln-His-β-Dap(BCMA)-Nle-c[Glu-Hyp-D-Phe-Arg-Trp-Lys]-NH₂

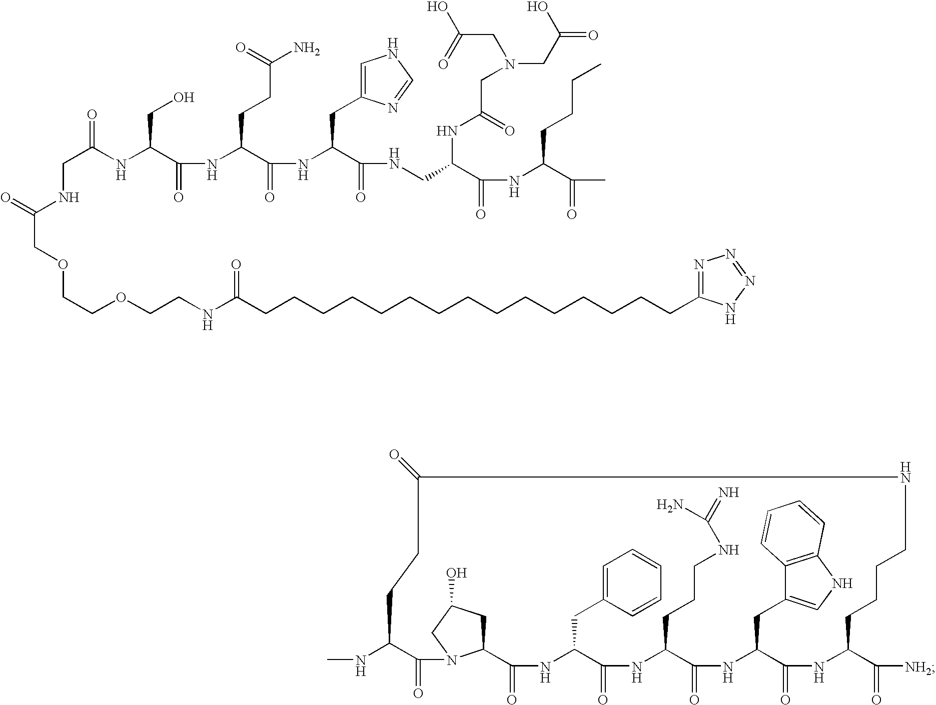

(2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-Lys(biscarboxymethyl)-Nle-c[Glu-Hyp-D-Phe-Arg-Trp-Lys]-NH₂

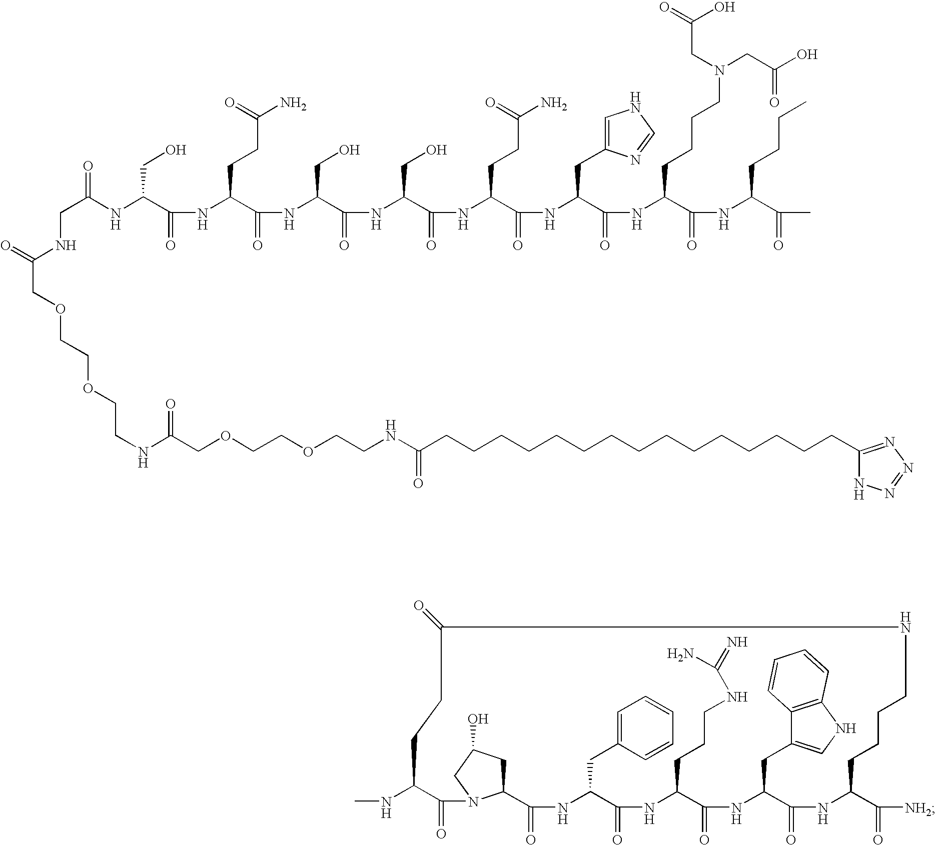

(2-{2-[16-(Tetrazol-5-yl)hexadecanoylamino]ethoxy}ethoxy)acetyl-Gly-Ser-Gln-Ser-Dap(BCMA)-Nle-c[Glu-Hyp-D-Phe-Arg-Trp-Lys]-NH₂

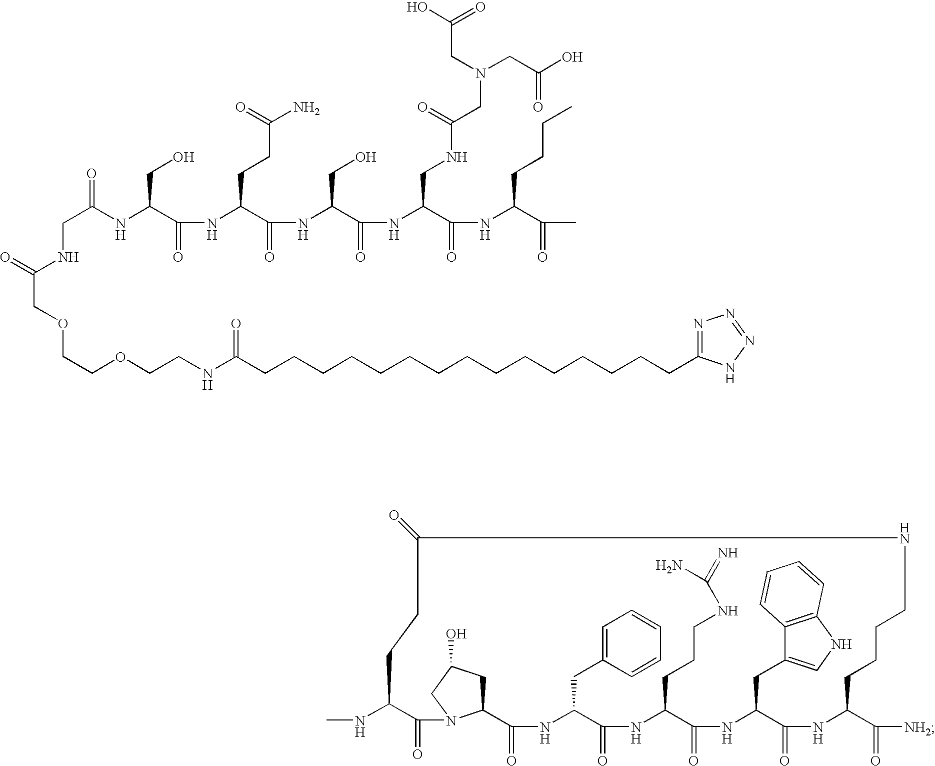

(2-{2-[16-(Tetrazol-5-yl)hexadecanoylamino]ethoxy}ethoxy)acetyl-Gly-Ser-Gln-Ser-Lys(biscarboxymethyl)-Nle-c[Glu-Hyp-D-Phe-Arg-Trp-Lys]-NH₂

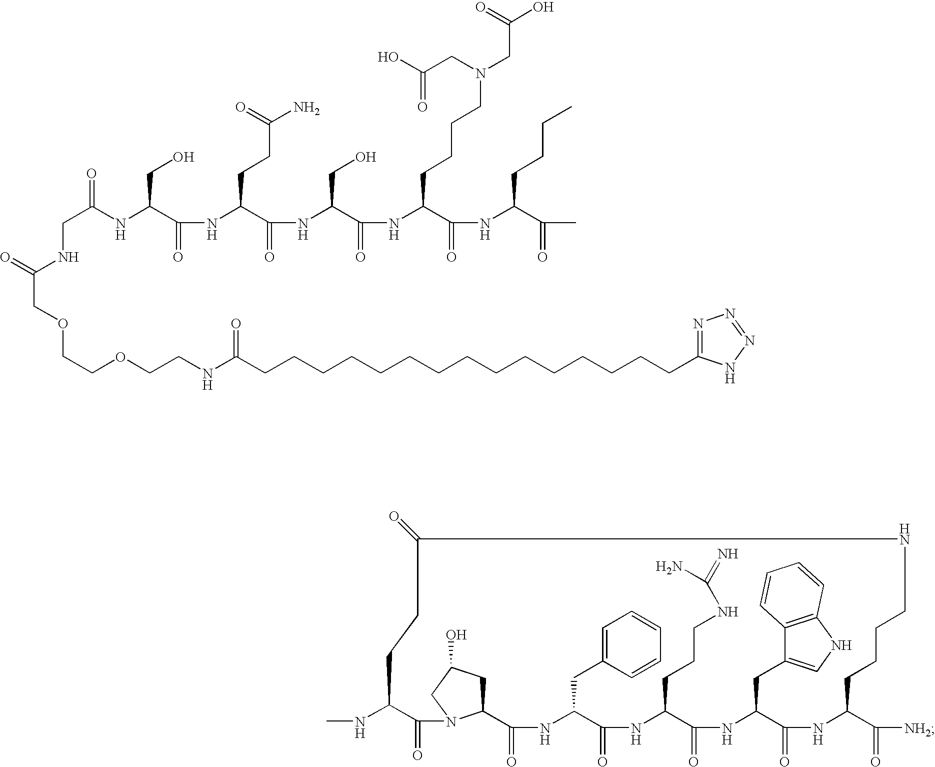

(2-{2-[16-(Tetrazol-5-yl)hexadecanoylamino]ethoxy}ethoxy)acetyl-Gly-Ser-Gln-His-Dap(biscarboxymethyl)-Nle-c[Glu-Hyp-D-Phe-Arg-Trp-Lys]-NH₂

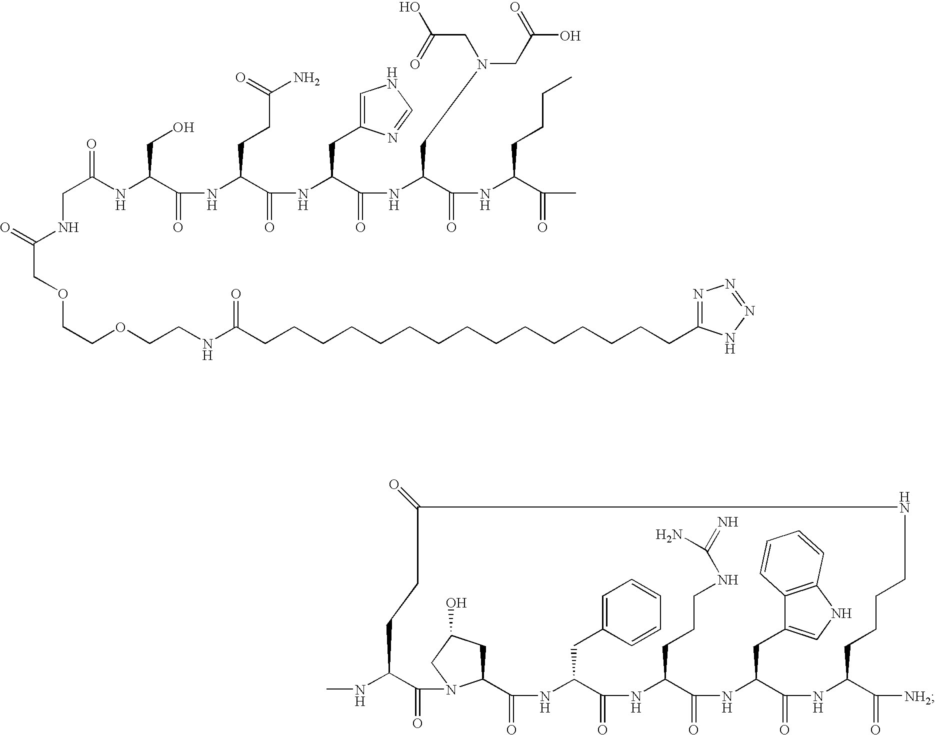

(2-{2-[16-(Tetrazol-5-yl)hexadecanoylamino]ethoxy}ethoxy)acetyl-Gly-Ser-Gln-His-Lys(biscarboxymethyl)-Nle-c[Glu-Hyp-D-Phe-Arg-Trp-Lys]-NH₂

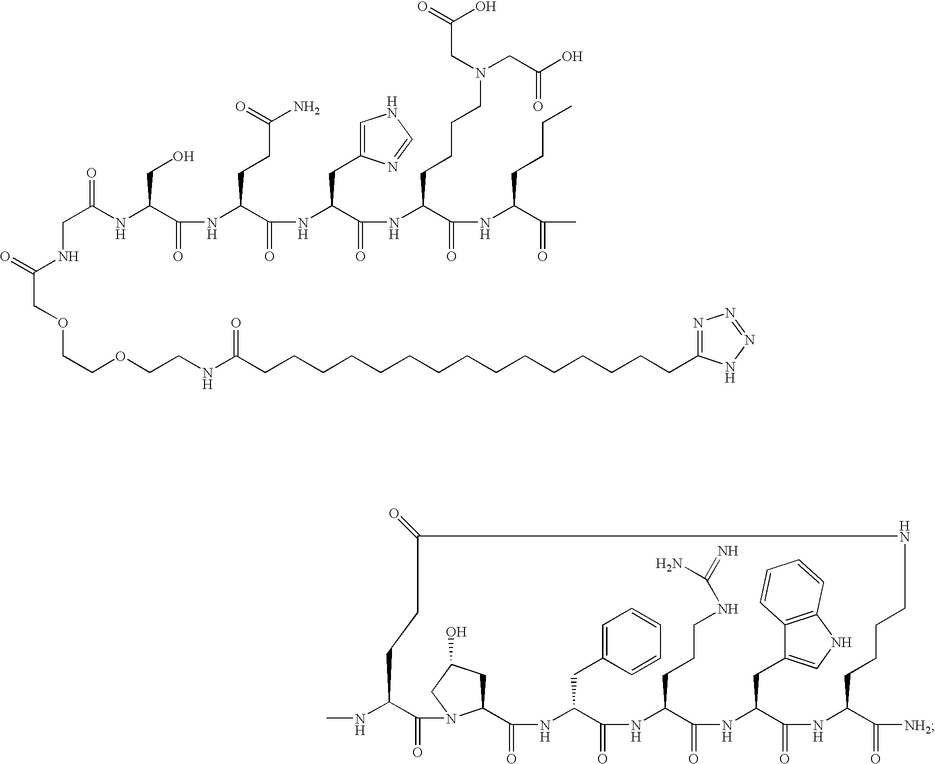

(2-{2-[16-(Tetrazol-5-yl)hexadecanoylamino]ethoxy}ethoxy)acetyl-Gly-Ser-Gln-His-Orn(BCMA)-Nle-c[Glu-Hyp-D-Phe-Arg-Trp-Lys]-NH₂

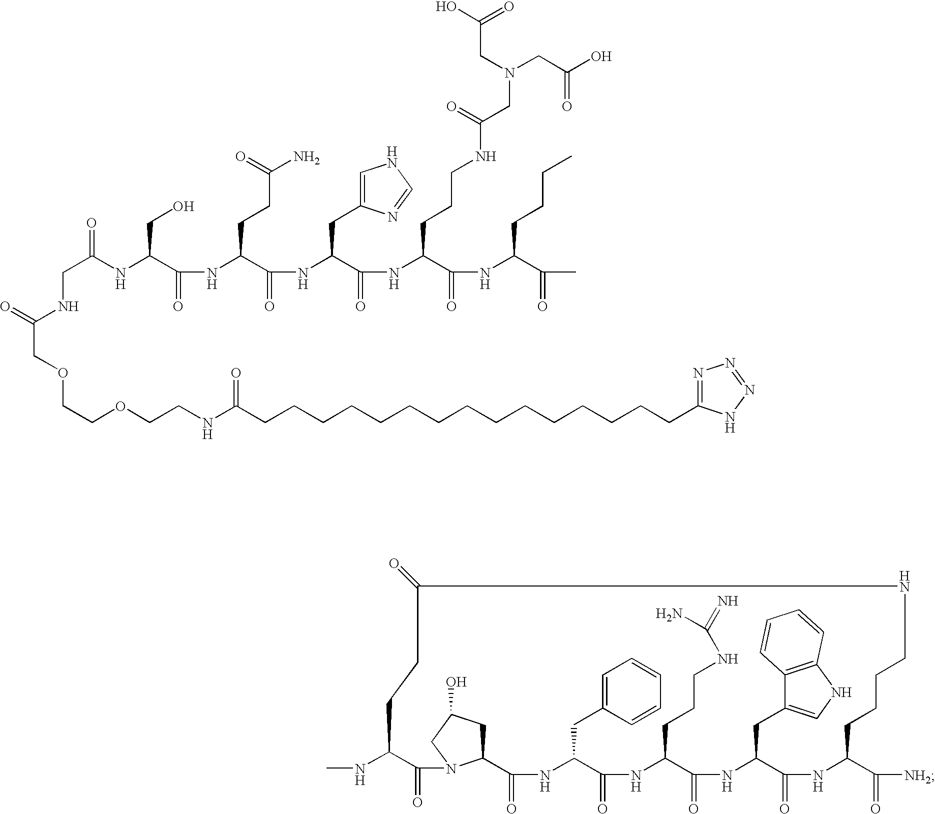

(2-{2-[16-(Tetrazol-5-yl)hexadecanoylamino]ethoxy}ethoxy)acetyl-Gly-Ser-Gln-His-Dap(BCMA)-Nle-c[Glu-Hyp-D-Phe-Arg-Trp-Lys]-NH₂

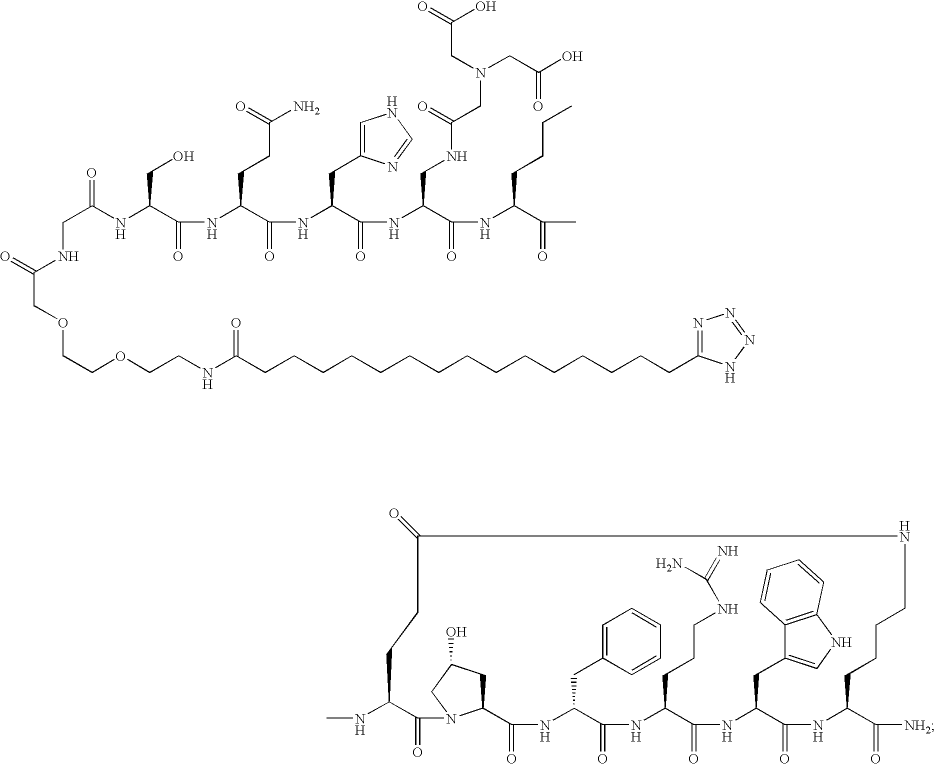

(2-{2-[16-(Tetrazol-5-yl)hexadecanoylamino]ethoxy}ethoxy)acetyl-Gly-Ser-Gln-His-Dab(BCMA)-Nle-c[Glu-Hyp-D-Phe-Arg-Trp-Lys]-NH₂

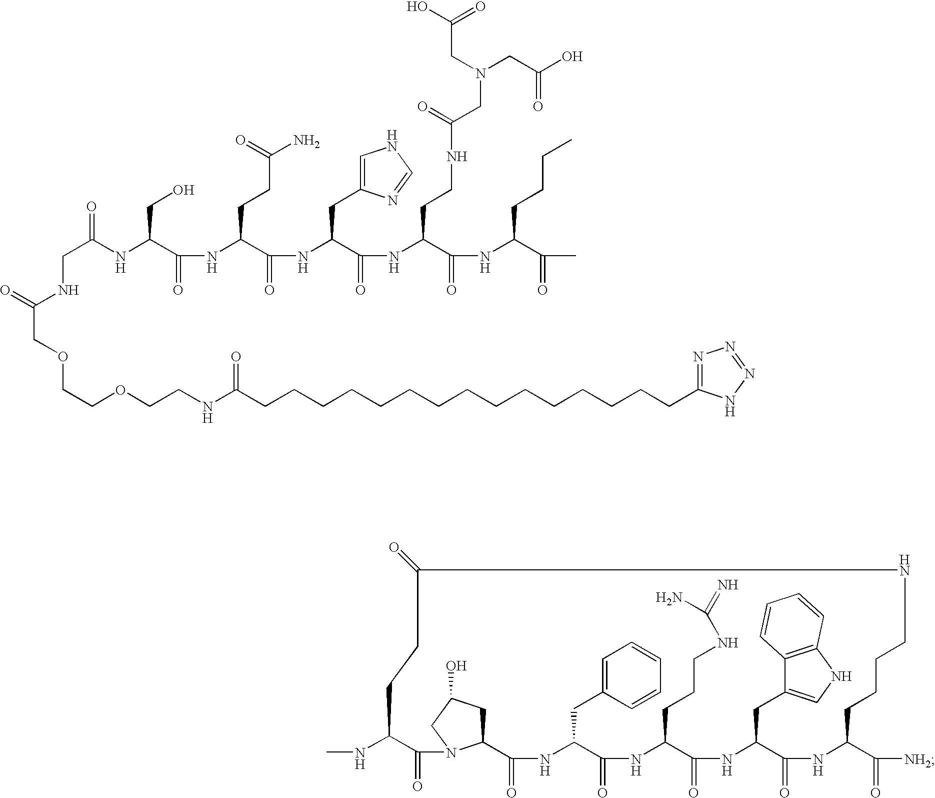

[2-(2-{4-[16-(Tetrazol-5-yl)hexadecanoylsulfamoyl]butanoylamino}ethoxy)ethoxy]acetyl-Gly-Ser-Gln-His-Dap(BCMA)-Nle-c[Glu-Hyp-D-Phe-Arg-Trp-Lys]-NH2

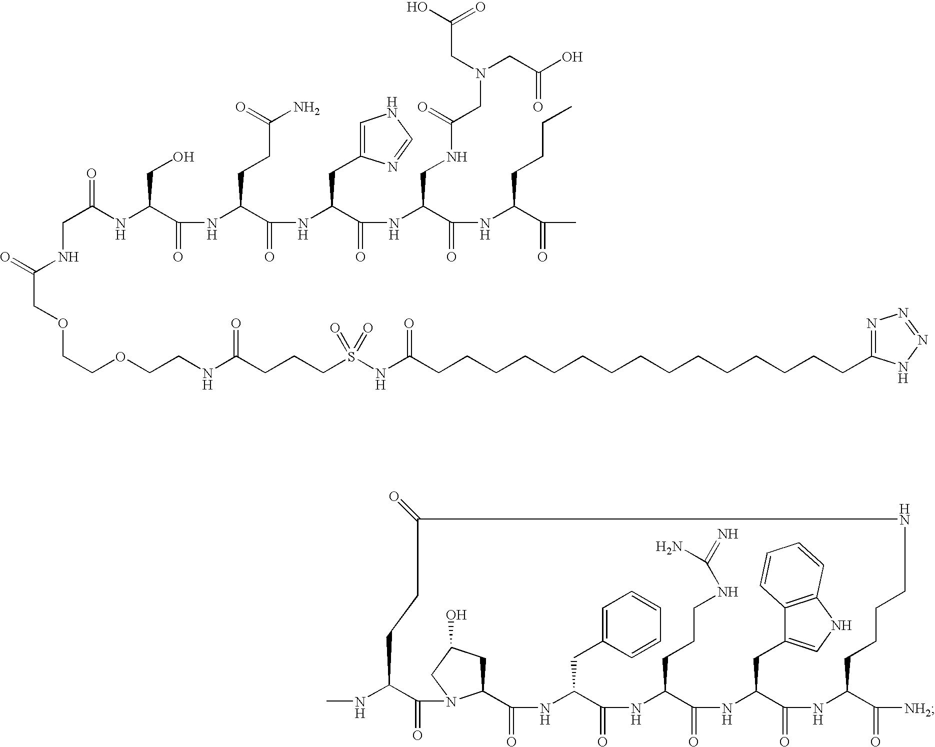

(2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-β-Ala-Lys(biscarboxymethyl)-Nle-c[Glu-Hyp-D-Phe-Arg-Trp-Lys]-NH₂

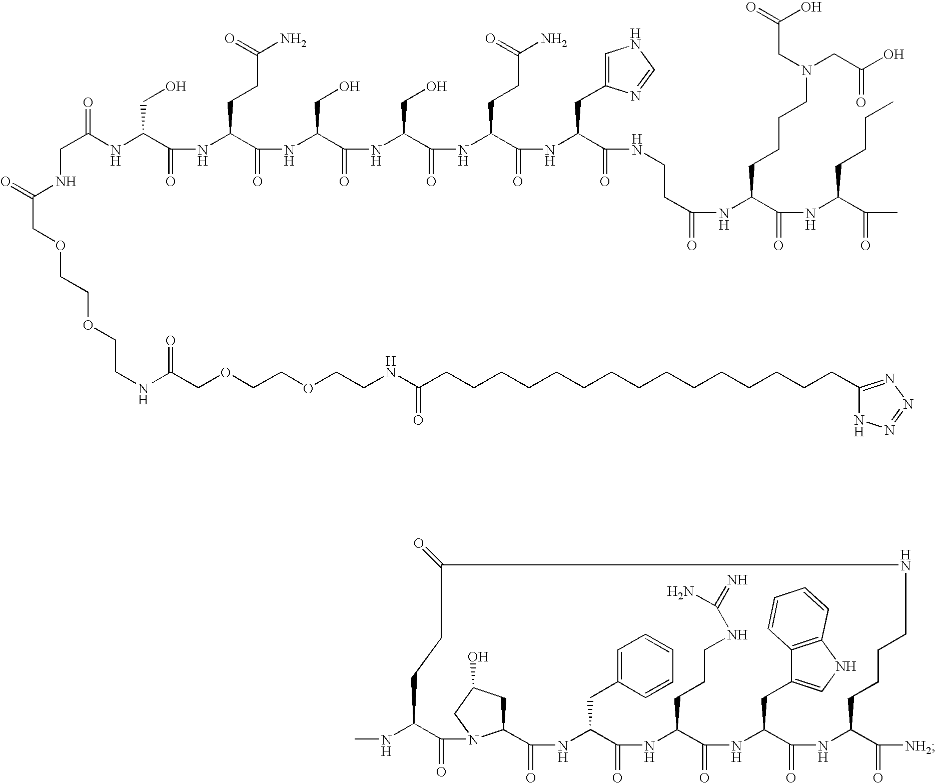

{2-[2-(15-Carboxypentadecanoylamino)ethoxy]ethoxy}acetyl-Gly-Ser-Gln-His-Dap(BCMA)-Nle-c[Glu-Hyp-D-Phe-Arg-Trp-Lys]-NH2

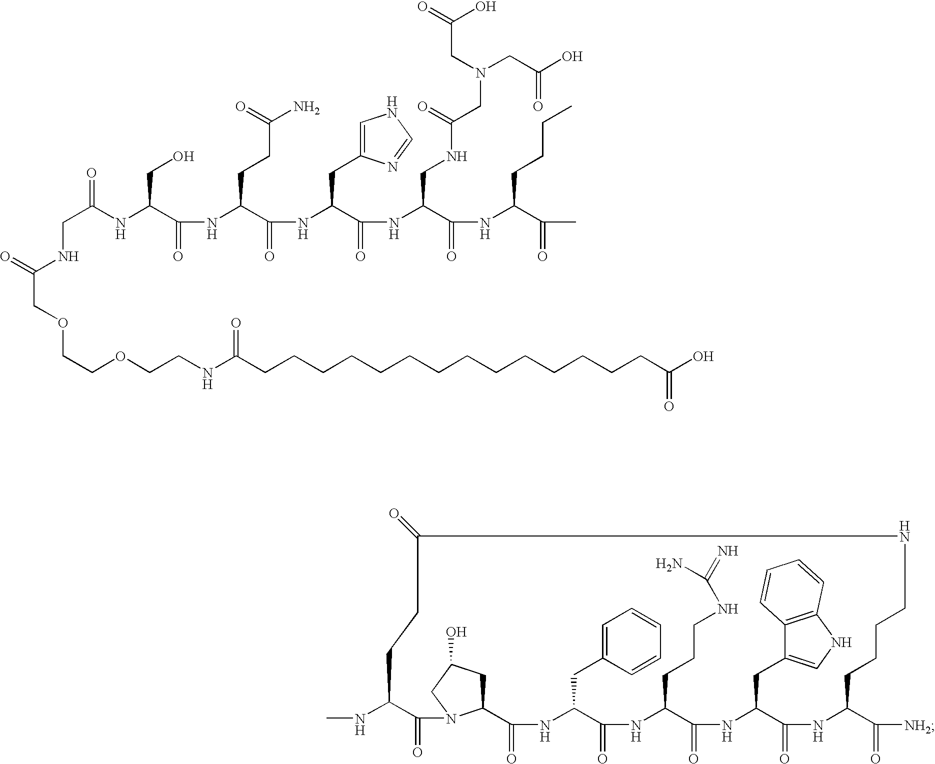

(2-{2-[2-(2-{2-[2-(2-{2-[2-(2-{2-β6-(Tetrazol-5-yl)hexadecanoylamino]ethoxy}ethoxy)acetylamino]ethoxy}ethoxy)acetylamino]ethoxy}ethoxy)acetylamino]ethoxy}ethoxy)acetyl-Gly-Ser-Gln-Ser-Dap(BCMA)-Nle-c[Glu-Hyp-D-Phe-Arg-Trp-Lys]-NH₂

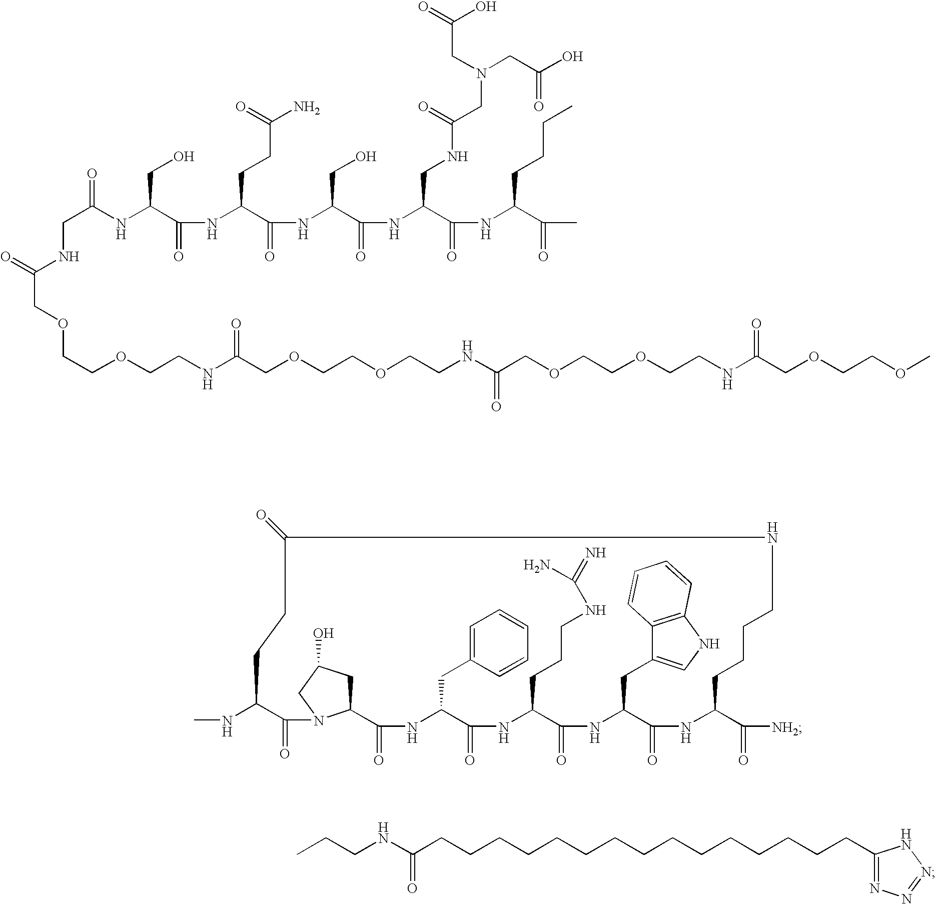

(2-{2-[16-(Tetrazol-5-yl)hexadecanoylamino]ethoxy}ethoxy)acetyl-Gly-Ser-Gln-Ser-Lys(BCMA)-Nle-c[Glu-Hyp-D-Phe-Arg-Trp-Lys]-NH₂

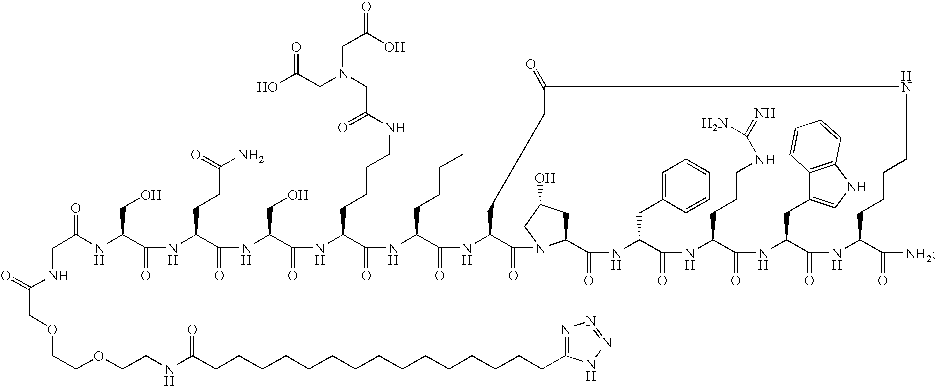

(2-{2-[2-(2-{2-[2-(2-{2-[2-(2-{2-[16-(Tetrazol-5-yl)hexadecanoylamino]ethoxy}ethoxy)acetylamino]ethoxy}ethoxy)acetylamino]ethoxy}ethoxy)acetylamino]ethoxy}ethoxy)acetyl-Gly-Ser-Gln-His-Dap(BCMA)-Nle-c[Glu-Hyp-D-Phe-Arg-Trp-Lys]-NH₂

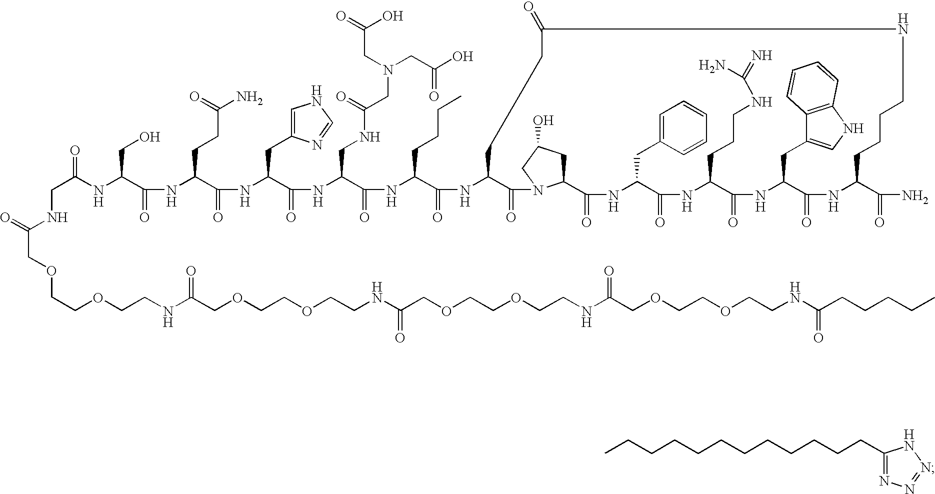

(2-{2-[16-(Tetrazol-5-yl)hexadecanoylamino]ethoxy}ethoxy)acetyl-Glu-Ser-Gln-His-Dap(BCMA)-Nle-c[Glu-Hyp-D-Phe-Arg-Trp-Lys]-NH₂

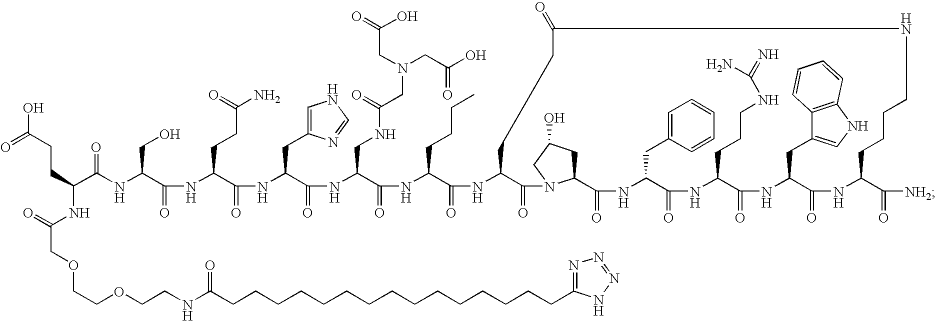

(2-{2-[2-(2-{2-[(S)-4-Carboxy-4-(17-carboxyheptadecanoylamino)butanoylamino]ethoxy}ethoxy)acetylamino]ethoxy}ethoxy)acetyl-Gly-Ser-Gln-His-Dap(BCMA)-Nle-c[Glu-Hyp-D-Phe-Arg-Trp-Lys]-NH₂

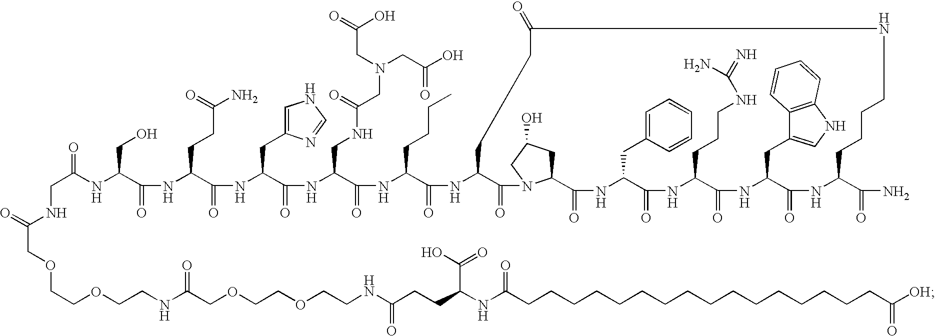

(2-{2-[16-(Tetrazol-5-yl)hexadecanoylamino]ethoxy}ethoxy)acetyl-Gly-Ser-Gln-Tyr-Dap(BCMA)-Nle-c[Glu-Hyp-D-Phe-Arg-Trp-Lys]-NH₂

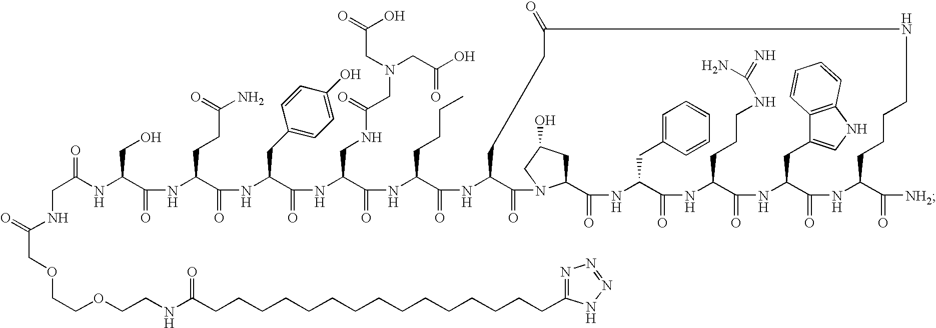

(2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-β-Ala-Lys(biscarboxymethyl)-Nle-c[Glu-Hyp-D-Phe-Arg-Trp-Lys]-NH₂

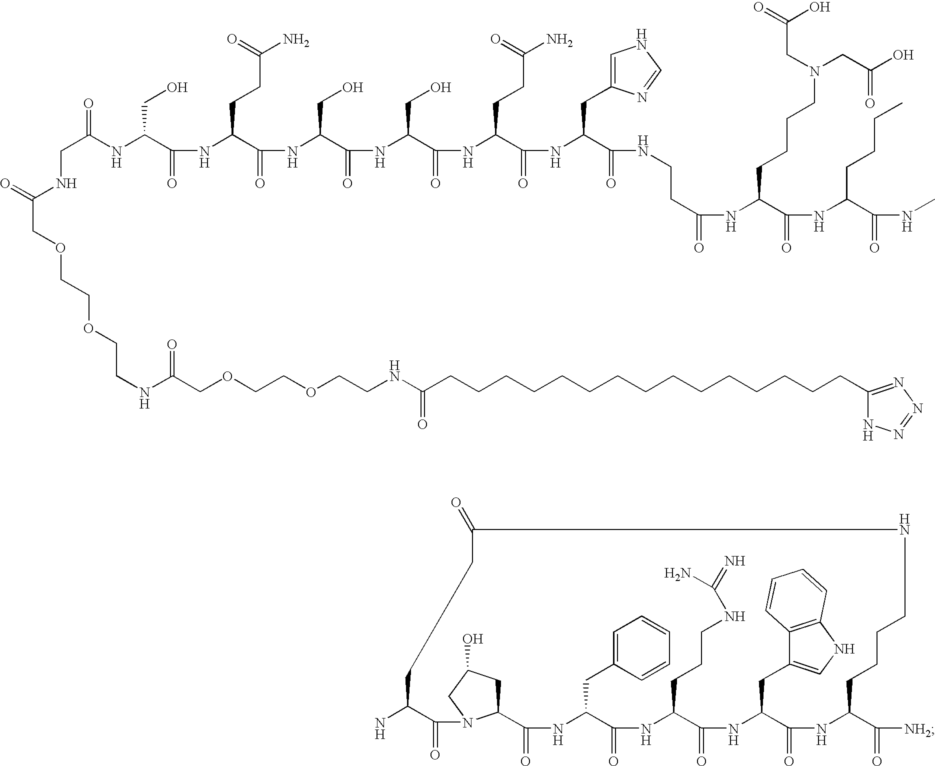

{2-[2-(15-Carboxypentadecanoylamino)ethoxy]ethoxy}acetyl-Gly-Ser-Gln-Ser-Lys(BCMA)-Nle-c[Glu-Hyp-D-Phe-Arg-Trp-Lys]-NH₂

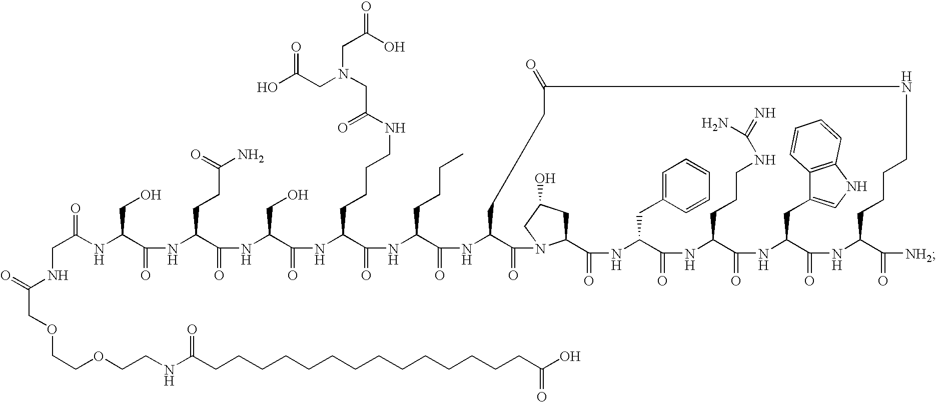

{2-[2-(2-{2-[2-(19-Carboxynonadecanoylamino)ethoxy]ethoxy}acetylamino)ethoxy]ethoxy}acetyl-Gly-D-Ser-Gln-Ser-Ser-Gln-His-Lys(biscarboxymethyl)-β-Ala-Nle-c[Glu-Hyp-D-Phe-Arg-Trp-Lys]-NH₂

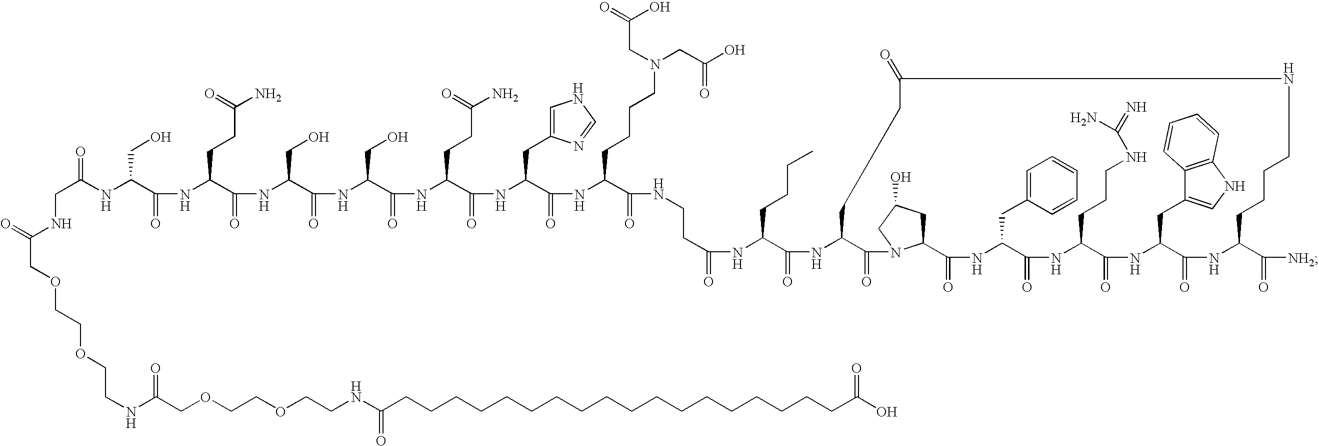

and
(2-{2-[16-(Tetrazol-5-yl)hexadecanoylamino]ethoxy}ethoxy)acetyl-Gly-Ser-Gln-Tyr-Dap(biscarboxymethyl)-Nle-c[Glu-Hyp-D-Phe-Arg-Trp-Lys]-NH₂

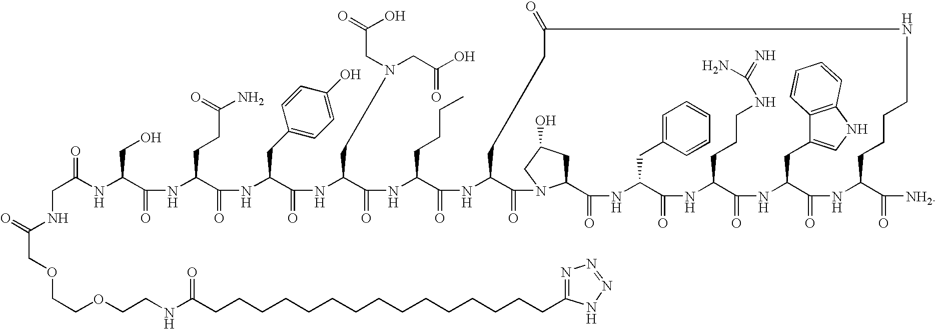

The present invention also encompasses combinations of two or more embodiments of compounds of the invention as outlined above.

In one aspect of the present invention, the compound of the invention is an agonist of a melanocortin receptor, notably an agonist of MC4. In another aspect of the invention, the compound is a selective agonist of MC4. In this context, selectivity is to be understood in relation to the activity of the compound with respect to MC1, MC3 and/or MC5. If a compound is a significantly more potent as a MC4 agonist than as a MC1, MC3 and/or MC5 agonist, it is deemed to be a selective MC4 agonist. The binding affinity of a compound with respect to MC1, MC3, MC5 and MC4 may be determined by comparing the Ki from an MC1, MC3 or MC5 binding assay as described below under “Assay IV” (MC1), “Assay VIII” (MC3) and “Assay IX” (MC5), respectively, with Ki from an MC4 binding assay as described below under “Assay V” (MC4). If a compound is more than 10 times, such as more than 50 times, e.g. more than 100 times more potent with respect to MC4 than with respect to MC1, it is deemed to be a selective MC4 agonist with respect to MC1. If a compound is more than 10 times, such as more than 50 times, e.g. more than 100 times more potent with respect to MC4 than with respect to MC3, it is deemed to be a selective MC4 agonist with respect to MC3. If a compound is more than 10 times, such as more than 50 times, e.g. more than 100 times more potent with respect to MC4 than with respect to MC5, it is deemed to be a selective MC4 agonist with respect to MC5. The agonistic potency of a compound with respect to MC3, MC4 and MC5 may be determined in functional assays as described in “Assay II” (MC3 and MC5), “Assay X” (MC3) and “Assay III” (MC4). If a compound is more than 10 times, such as more than 50 times, e.g. more than 100 times more potent with respect to MC4 than with respect to MC3, it is deemed to be a selective MC4 agonist with respect to MC3. If a compound is more than 10 times, such as more than 50 times, e.g. more than 100 times more potent with respect to MC4 than with respect to MC5, it is deemed to be a selective MC4 agonist with respect to MC5. In a particular aspect, the compound of the present invention is a selective MC4 agonist with respect to MC1, with respect to MC3, with respect to MC5, with respect to MC1 and MC3, with respect to MC1 and MC5, with respect to MC3 and MC5 or with respect to MC1, MC3 and MC5.

In a further aspect of the present invention, the compound of the present invention is both a selective MC3 agonist and a selective MC4 agonist. In this context, a compound is deemed to be a selective MC3 and MC4 agonist if it is significantly more potent as an agonist towards MC3 and MC4 than as an agonist toward MC1 and MC5. The selectivity of a compound with respect to MC1 and MC3 may be determined by comparing the binding affinity determined for MC1 as described in “Assay IV” with the binding affinity for MC3 determined as described in “Assay VIII”. If the binding affinity of a compound is more than 10 times, such as more than 50 times, e.g. more than 100 times greater with respect to MC3 than with respect to MC1, it is deemed to be a selective MC3 agonist with respect to MC1. The selectivity of a compound with respect to MC3 and MC5 may be determined by comparing the affinity determined as described in “Assay VIII and IX”. If the binding affinity of a compound is more than 10 times, such as more the 50 times, e.g. more than 100 times greater with respect to MC3 than with respect to MC5, it is deemed to be a selective MC3 agonist with respect to MC5. The MC4 selectivity of a compound with respect to MC3 and MC5 is determined as discussed above.

Compounds of the present invention may exert a protracted effect, i.e. the period of time in which they exert a biological activity is prolonged. Effect is defined as being protracted when a compound significantly reduces food intake in the period from 24 hours to 48 hours in test animals compared to the food intake in the same time period in the vehicle-treated control group of animals in “Assay I”. Alternatively, a protracting effect may be evaluated in an indirect albumin-binding assay, in which Ki determined for binding in the presence of ovalbumin is compared with the EC₅₀ value determined in the presence of HSA [see Assay VII in the “Pharmacological methods” section (vide infra) for a description of a suitable assay procedure].

Compounds of the present invention modulate melanocortin receptors, and they are therefore believed to be particularly suited for the treatment of diseases or states which can be treated by a modulation of melanocortin receptor activity. In particular, compounds of the present invention are believed to be suited for the treatment of diseases or states via activation of MC4.

Among further aspects or embodiments of the present invention are the following:
65. A method of delaying the progression from IGT to type 2 diabetes, comprising administering to a patient in need thereof an effective amount of a compound according to any of embodiments 1-64 (vide supra), optionally in combination with one or more additional therapeutically active compounds.
66. A method of delaying the progression from non-insulin-requiring type 2 diabetes to insulin-requiring type 2 diabetes, comprising administering to a patient in need thereof an effective amount of a compound according to any of embodiments 1-64, optionally in combination with one or more additional therapeutically active compounds.
67. A method of treating obesity or preventing overweight, comprising administering to a patient in need thereof an effective amount of a compound according to any of embodiments 1-64, optionally in combination with one or more additional therapeutically active compounds.
68. A method of regulating appetite, comprising administering to a patient in need thereof an effective amount of a compound according to any of embodiments 1-64, optionally in combination with one or more additional therapeutically active compounds.
69. A method of inducing satiety, comprising administering to a patient in need thereof an effective amount of a compound according to any of embodiments 1-64, optionally in combination with one or more additional therapeutically active compounds.
70. A method of preventing weight gain after successfully having lost weight, comprising administering to a patient in need thereof an effective amount of a compound according to any of embodiments 1-64, optionally in combination with one or more additional therapeutically active compounds.
71. A method of increasing energy expenditure, comprising administering to a patient in need thereof an effective amount of a compound according to any of embodiments 1-64, optionally in combination with one or more additional therapeutically active compounds.

Among yet further aspects or embodiments of the present invention are the following:
72. A method of treating a disease or state related to overweight or obesity, comprising administering to a patient in need thereof an effective amount of a compound according to any of embodiments 1-64, optionally in combination with one or more additional therapeutically active compounds.
73. A method of treating bulimia, comprising administering to a patient in need thereof an effective amount of a compound according to any of embodiments 1-64, optionally in combination with one or more additional therapeutically active compounds.
74. A method of treating a disease or state selected from atherosclerosis, hypertension, type 2 diabetes, impaired glucose tolerance (IGT), dyslipidemia, coronary heart disease, gallbladder disease, gall stone, osteoarthritis, cancer, sexual dysfunction and risk of premature death, comprising administering to a patient in need thereof an effective amount of a compound according to any of embodiments 1-64, optionally in combination with one or more additional therapeutically active compounds.

Compounds of the present invention may be suited for the treatment of diseases in obese or overweight patients. Accordingly, a yet further aspect or embodiment of the invention relates to the following:
75. A method of treating, in an obese patient, a disease or state selected from type 2 diabetes, IGT, dyslipidemia, coronary heart disease, gallbladder disease, gall stone, osteoarthritis, cancer, sexual dysfunction, risk of premature death, neuronal protection, effect in ischemic heart disease or anti-inflammatory effects comprising administering to an obese patient in need thereof an effective amount of a compound according to any of embodiments 1-64, optionally in combination with one or more additional therapeutically active compounds.

Yet further aspects or embodiments of the invention relate to:
76. A method according to any of embodiments 65-75 (vide supra), wherein said additional therapeutically active compound is selected from antidiabetic agents, antihyperlipidemic agents, antiobesity agents, antihypertensive agents and agents for the treatment of complications resulting from, or associated with, diabetes.
77. A method according to any of embodiments 65-76, wherein said compound according to any of embodiments 1-64 is administered to said patient in a unit dosage form comprising from about 0.05 mg to about 1000 mg of said compound.
78. A method according to any of embodiments 65-77, wherein said compound according to any of embodiments 1-64 is administered to said patient, once daily.
79. A method according to any of embodiments 65-77, wherein said compound according to any of embodiments 1-64 is administered to said patient once weekly.
80. A method of activating MC4 in a subject, the method comprising administering to said subject an effective amount of a compound according to any of embodiments 1-64.
81. A method according to any of embodiments 65-75, wherein said compound according to any of embodiments 1-64 is administered parenterally, orally, nasally, buccally or sublingually.
82. A method according to any of embodiments 65-75, wherein said compound according to any of embodiments 1-64 is administered parenterally or sublingually.

Another aspect or embodiment of the invention relates to:
83. A pharmaceutical composition comprising a compound according to any of embodiments 1-64 and one or more excipients. The compound of the invention in such a pharmaceutical composition may optionally be present in combination with one or more additional therapeutically active compounds or substances and/or together with one or more pharmaceutically acceptable carriers or excipients. A pharmaceutical composition of the invention may suitably be in unit dosage form comprising from about 0.05 mg to about 1000 mg, such as from about 0.1 mg to about 500 mg, e.g. from about 0.5 mg to about 200 mg, of a compound of the invention.

Yet another aspect or embodiment of the invention relates to the following:
84. A compound according to any of embodiments 1-64 for use in therapy.
85. The use of a compound according to any of embodiments 1-64 in the manufacture of a medicament for delaying the progression from impaired glucose tolerance (IGT) to type 2 diabetes; delaying the progression from type 2 diabetes to insulin-requiring diabetes; treating obesity or preventing overweight; regulating appetite; inducing satiety; preventing weight regain after successful weight loss; increasing energy expenditure; treating a disease or state related to overweight or obesity; treating bulimia; treating binge-eating; treating atherosclerosis, hypertension, type 2 diabetes, IGT, dyslipidemia, coronary heart disease, gallbladder disease, gall stone, osteoarthritis, cancer, sexual dysfunction, hypthalamic amenorrhea or risk of premature death; or treating, in an obese patient, a disease or state selected from type 2 diabetes, IGT, dyspilidemia, coronary heart disease, gallbladder disease, gall stone, osteoarthritis, cancer, sexual dysfunction, risk of premature death; for providing neuronal protection, for having an effect on ischemic heart disease or anti-inflammatory effects and for the treatment of autoimmune diseases, e.g. multiple sclerosis.

Compounds of the invention that act as MC4 agonists could have a positive effect on insulin sensitivity, on drug abuse (by modulating the reward system) and on hemorrhagic shock. Furthermore, MC3 and MC4 agonists have antipyretic effects, and both have been suggested to be involved in peripheral nerve regeneration. MC4 agonists are also known to reduce stress response. In addition to treating drug abuse, treating or preventing hemorrhagic shock, and reducing stress response, compounds of the invention may also be of value in treating alcohol abuse, treating stroke, treating ischemia and protecting against neuronal damage.

As already indicated, in all of the therapeutic methods or indications disclosed above, the compound of the present invention may be administered alone. However, it may also be administered in combination with one or more additional therapeutically active agents, substances or compounds, either sequentially or concomitantly.

A typical dosage of a compound of the invention when employed in a method according to the present invention is in the range of from about 0.001 to about 100 mg/kg body weight per day, preferably from about 0.01 to about 10 mg/kg body weight, more preferably from about 0.01 to about 5 mg/kg body weight per day, e.g. from about 0.05 to about 10 mg/kg body weight per day or from about 0.03 to about 5 mg/kg body weight per day administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the invention may conveniently be formulated in unit dosage form using techniques well known to those skilled in the art. A typical unit dosage form intended for oral administration one or more times per day, such as from one to three times per day, may suitably contain from about 0.05 to about 1000 mg, preferably from about 0.1 to about 500 mg, such as from about 0.5 to about 200 mg of a compound of the invention.

Compounds of the invention comprise compounds that are believed to be well-suited to administration with longer intervals than, for example, once daily, thus, appropriately formulated compounds of the invention may be suitable for, e.g., twice-weekly or once-weekly administration by a suitable route of administration, such as one of the routes disclosed herein.

As described above, compounds of the present invention may be administered or applied in combination with one or more additional therapeutically active compounds or substances, and suitable additional compounds or substances may be selected, for example, from antidiabetic agents, antihyperlipidemic agents, antiobesity agents, antihypertensive agents and agents for the treatment of complications resulting from, or associated with, diabetes.

Suitable antidiabetic agents include insulin, insulin derivatives or analogues, GLP-1 (glucagon like peptide-1) derivatives or analogues [such as those disclosed in WO 98/08871 (Novo Nordisk A/S), which is incorporated herein by reference, or other GLP-1 analogues such as exenatide (Byetta, Eli Lilly/Amylin; AVE0010, Sanofi-Aventis), taspoglutide (Roche), albiglutide (Syncria, GlaxoSmithKline), amylin, amylin analogues (e.g. Symlin™/Pramlintide) as well as orally active hypoglycemic agents.

Suitable orally active hypoglycemic agents include: metformin, imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk A/S) which are incorporated herein by reference; potassium channel openers such as ormitiglinide; potassium channel blockers such as nateglinide or BTS-67582; glucagon receptor antagonists such as those disclosed in WO 99/01423 and WO 00/39088 (Novo Nordisk A/S and Agouron Pharmaceuticals, Inc.), all of which are incorporated herein by reference; GLP-1 receptor agonists such as those disclosed in WO 00/42026 (Novo Nordisk A/S and Agouron Pharmaceuticals, Inc.), which are incorporated herein by reference; amylin analogues (agonists on the amylin receptor); DPP-IV (dipeptidyl peptidase-IV) inhibitors; PTPase (protein tyrosine phosphatase) inhibitors; glucokinase activators, such as those described in WO 02/08209 to Hoffmann La Roche; inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as anti-hyperlipidemic agents and antilipidemic agents; compounds lowering food intake; as well as PPAR (peroxisome proliferator-activated receptor) agonists and RXR (retinoid X receptor) agonists such as ALRT-268, LG-1268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-1037 or T 174, or the compounds disclosed in WO 97/41097 (DRF-2344), WO 97/41119, WO 97/41120, WO 00/41121 and WO 98/45292 (Dr. Reddy's Research Foundation), the contents of all of which are incorporated herein by reference.

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), the contents of all of which are incorporated herein by reference.

Still further examples of suitable additional therapeutically active substances include: α-glucosidase inhibitors, e.g. voglibose, emiglitate, miglitol or acarbose; glycogen phosphorylase inhibitors, e.g. the compounds described in WO 97/09040 (Novo Nordisk A/S); glucokinase activators; agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y receptor 1 and/or 5) antagonists, MC3 (melanocortin receptor 3) agonists, MC3 antagonists, MC4 (melanocortin receptor 4) agonists, orexin receptor antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MC1 (melanocortin receptor 1) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, 5HT6 agonists, 5HT2c agonists, bombesin agonists, galanin antagonists, growth hormone, growth factors such as prolactin or placental lactogen, growth hormone releasing compounds, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, the contents of all of which are incorporated herein by reference, exendin-4 analogues, GLP-1 analogues, ciliary neurotrophic factor, amylin analogues, peptide YY₃₋₃₆ (PYY3-36) (Batterham et al, Nature 418, 650-654 (2002)), PYY3-36 analogues, NPY Y2 receptor agonists, NPY Y4 receptor agonists and substances acting as combined NPY Y2 and NPY Y4 agonists, FGF21 and analogues thereof, μ-opioid receptor antagonists, oxyntomodulin or analogues thereof.

Further suitable antiobesity agents are bupropion (antidepressant), topiramate (anticonvulsant), ecopipam (dopamine D1/D5 antagonist) and naltrexone (opioid antagonist), and combinations thereof.

Among embodiments of suitable antiobesity agents for use in a method of the invention as additional therapeutically active substances in combination with a compound of the invention are leptin and analogues or derivatives of leptin.

Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include anti-hypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

In certain embodiments of the uses and methods of the present invention, the compound of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In the case, in particular, of administration of a compound of the invention, optionally in combination with one or more additional therapeutically active compounds or substances as disclosed above, for a purpose related to treatment or prevention of obesity or overweight, i.e. related to reduction or prevention of excess adiposity, it may be of relevance to employ such administration in combination with surgical intervention for the purpose of achieving weight loss or preventing weight gain, e.g. in combination with bariatric surgical intervention. Examples of frequently used bariatric surgical techniques include, but are not limited to, the following: vertical banded gastroplasty (also known as “stomach stapling”), wherein a part of the stomach is stapled to create a smaller pre-stomach pouch which serves as a new stomach; gastric banding, e.g. using an adjustable gastric band system (such as the Swedish Adjustable Gastric Band (SAGB), the LAP-BAND™ or the MIDband™), wherein a small pre-stomach pouch which is to serve as a new stomach is created using an elastomeric (e.g. silicone) band which can be adjusted in size by the patient; and gastric bypass surgery, e.g. “Roux-en-Y” bypass wherein a small stomach pouch is created using a stapler device and is connected to the distal small intestine, the upper part of the small intestine being reattached in a Y-shaped configuration.

Another technique which is within the scope of the term “bariatric surgery” and variants thereof (e.g. “weight-loss surgery”, “weight-loss surgical intervention” “weight-loss surgical procedure”, “bariatric surgical intervention”, “bariatric surgical procedure” and the like) as employed in the context of the present invention is gastric balloon surgery, wherein an inflatable device resembling a balloon is introduced into the stomach and then inflated, the purpose being to reduce the accessible volume within the stomach to create a sensation of satiety in the patient at an earlier stage than normal during food intake, and thereby cause a reduction in food intake by the patient.

All of the above-mentioned techniques are in principle reversible. Non-limiting examples of additional, irreversible and consequently generally less frequently employed techniques of relevance in the present context include biliopancreatic diversion and sleeve gastrectomy (the latter of which may also be employed in conjunction with duodenal switch), both of which entail surgical resection of a substantial portion of the stomach.

The administration of a compound of the invention (optionally in combination with one or more additional therapeutically active compounds or substances as disclosed above) may take place for a period prior to carrying out the bariatric surgical intervention in question and/or for a period of time subsequent thereto. In many cases it may be preferable to begin administration of a compound of the invention after bariatric surgical intervention has taken place.

The treatment of obesity might be possible by using long-acting melanocortin 4 receptor agonists (MC4 agonists) comprising a peptide part and an albumin binding fatty acid or alkyltetrazole chain as described in e.g. WO2007/009894, WO2008/087186 and WO2008/087187. These compounds have more basic than acidic residues, resulting in good water solubility at acidic pH, but poor solubility at neutral or weakly basic pH. Water solubility at pH from 6 to 9 is considered to be an advantage, since this could improve local tolerance and make it possible to combine the MC4 agonist with other drugs, soluble only at neutral to weakly basic pH.

The problem of solubility at neutral to weakly basic pH could not just be solved by incorporating several negatively charged residues into the peptide (for example three Glu residues in the N-terminal part), since this resulted in reduced MC4 receptor activity and poor reduction of food-intake in vivo. Surprisingly, the problem was solved by incorporating one of several novel synthetic amino acid residues containing a (bis-carboxymethyl)amino group at one certain position in the peptide. This group has both acidic and basic properties, thus making the compound more soluble at pH 7-8, but also sufficiently potent at the MC4 receptor. The compounds of the present invention are negatively charged and sufficiently water-soluble at neutral pH. The (bis-carboxymethyl)amino group is negatively charged at neutral pH and thus significantly contributes to the water-solubility of the compounds of the present invention.

The compounds of the present invention can be a water-soluble MC4 receptor agonist, for example with water-solubility of at least 0.2 mmol/l, at least 0.5 mmol/l, at least 2 mmol/l, at least 4 mmol/l, at least 8 mmol/l, at least 10 mmol/l, or at least 15 mmol/l, at pH 7.5.

The term “obesity” implies an excess of adipose tissue. When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity. In this context, obesity is best viewed as any degree of excess adipose tissue that imparts a health risk. The distinction between normal and obese individuals can only be approximated, but the health risk imparted by obesity is probably a continuum with increasing adipose tissue. However, in the context of the present invention, individuals with a body mass index (BMI=body weight in kilograms divided by the square of the height in meters) above 25 are to be regarded as obese.

The use of a prefix of the type “C_{x-y}” preceding the name of a radical, such as in C_{x-y}alkyl (e.g. C₆₋₂₀alkyl) is intended to indicate a radical of the designated type having from x to y carbon atoms.

The term “alkyl” as used herein refers to a straight-chain, branched and/or cyclic, saturated monovalent hydrocarbon radical.

The term “alkenyl” as used herein refers to a straight-chain, branched and/or cyclic, monovalent hydrocarbon radical comprising at least one carbon-carbon double bond. The term “alkynyl” as used herein refers to a straight-chain, branched and/or cyclic, monovalent hydrocarbon radical comprising at least one carbon-carbon triple bond, and it may optionally also comprise one or more carbon-carbon double bonds.

The term “alkylene” as used herein refers to a straight-chain, branched and/or cyclic, saturated bivalent hydrocarbon radical.

The term “alkenylene” as used herein refers to a straight-chain, branched and/or cyclic, bivalent hydrocarbon radical comprising at least one carbon-carbon double bond.

The term “alkynylene” as used herein refers to a straight-chain, branched and/or cyclic, bivalent hydrocarbon radical comprising at least one carbon-carbon triple bond, and it may optionally also comprise one or more carbon-carbon double bonds.

The term “alkoxy” as used herein is intended to indicate a radical of the formula —OR′, wherein R′ is alkyl as indicated above.

In the present context, the term “aryl” is intended to indicate a carbocyclic aromatic ring radical or a fused aromatic ring system radical wherein at least one of the rings is aromatic. Typical aryl groups include phenyl, biphenylyl, naphthyl, and the like. The term “halogen” is intended to indicate members of the 7^{th} main group of the periodic table of the elements, which includes fluorine, chlorine, bromine and iodine (corresponding to fluoro, chloro, bromo and iodo substituents, respectively).

The term “tetrazol-5-yl” is intended to indicate 1H-tetrazol-5-yl or 2H-tetrazol-5-yl.

In the present context, common rules for peptide nomenclature based on the three letter amino acid code apply, unless exceptions are specifically indicated. Briefly, the central portion of the amino acid structure is represented by the three letter code (e.g. Ala, Lys) and L-configuration is assumed, unless D-configuration is specifically indicated by “D-” followed by the three letter code (e.g. D-Ala, D-Lys). A substituent at the amino group replaces one hydrogen atom and its name is placed before the three letter code, whereas a C-terminal substituent replaces the carboxylic hydroxy group and its name appears after the three letter code. For example, “acetyl-Gly-Gly-NH₂” represents CH₃—C(═O)—NH—CH₂—C(═O)—NH—CH₂—C(═O)—NH₂. Unless indicated otherwise, amino acids with additional amino or carboxy groups in the side chains (such as Lys, Orn, Dap, Glu, Asp and others) are connected to their neighboring groups by amide bonds formed at the N-2 (α-nitrogen) atom and the C-1 (C═O) carbon atom.

When two amino acids are said to be bridged, it is intended to indicate that functional groups in the side chains of the two respective amino acids have reacted to form a covalent bond.

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist.

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As use herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid.

The amino acid abbreviations used in the present context have the following meanings:

| | |
|---|---|
| Ala | Alanine |
| β-Ala | 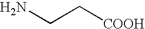 |
| Asn | Asparagine |
| Asn (alkyl) | 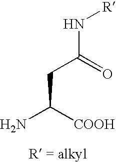 |
| Asn (aryl) | 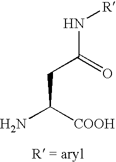 |
| Asp | aspartic acid |
| β-Asp | 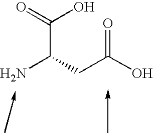 α-nitrogen and β-carboxy group form the amide bonds to the two neighboring residues |
| Arg | Arginine |
| Aze | (S)-Azetidine-2-carboxylic acid |
| Cha | 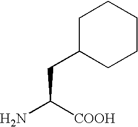 cyclohexylalanine |
| Cgl | 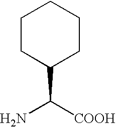 cyclohexylglycine |
| Cit | Citrulline |
| Cys | Cysteine |
| Dab | (S)-2,4-diaminobutyric acid |
| Dab (biscarboxymethyl) | 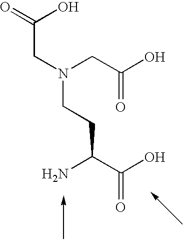 α-nitrogen and carboxy-carbon atom C-1 form the amide bonds to the two neighboring residues |
| Dab (BCMA) | 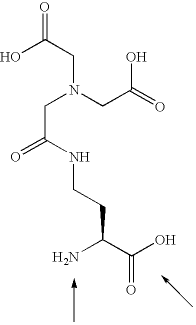 α-nitrogen and carboxy-carbon atom C-1 form the amide bonds to the two neighboring residues |
| Dap | (S)-2,3-diaminopropionic acid |
| β-Dap | 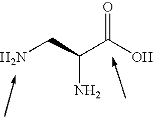 β-nitrogen and carboxy group form the amide bonds to the two neighboring residues |
| Dap (biscarboxymethyl) | 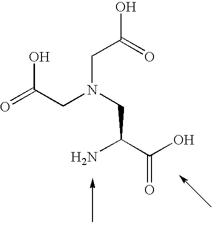 α-nitrogen and carboxy-carbon atom C-1 form the amide bonds to the two neighboring residues |
| Dap (BCMA) | 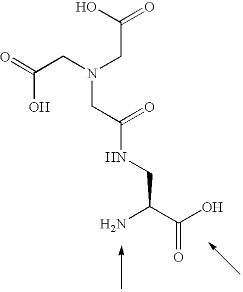 α-nitrogen and carboxy-carbon atom C-1 form the amide bond to the two neighboring residues |
| β-Dap (BCMA) | 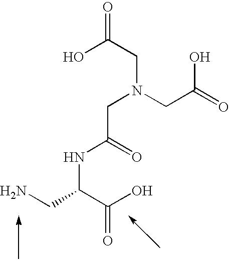 β-nitrogen and carboxy-carbon atom C-1 form the amide bonds to the two neighboring residues |
| D-β-Asp | 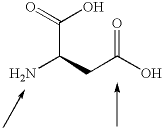 α-nitrogen and β-carboxy group form the amide bonds to the two neighboring residues |
| D-Dap | (R)-2,3-diaminopropionic acid |
| D-γ-Glu | 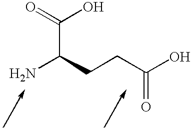 α-nitrogen and γ-carboxy group form the amide bonds to the two neighboring residues |
| D-Phe | 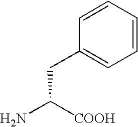 |
| Gln | Glutamine |
| Gln (alkyl) | 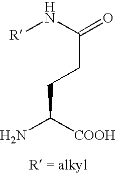 |
| Gln (aryl) | 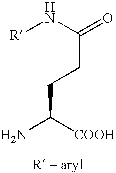 |
| Glu | glutamic acid |
| γ-Glu | 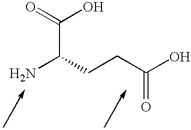 α-nitrogen and γ-carboxy group form the amide bonds to the two neighboring residues |
| Gly | Glycine |
| His | Histidine |
| homoArg | 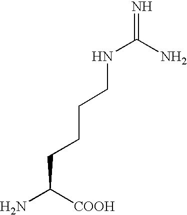 homo-arginine |
| homoCys | 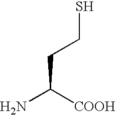 homo-cysteine |
| homoLys | 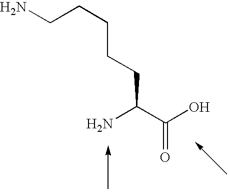 α-nitrogen and carboxy group form the amide bonds to the two neighboring residues |
| homoLys (biscarboxymethyl) | 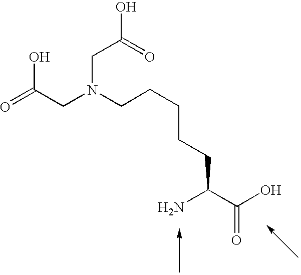 α-nitrogen and carboxy-carbon atom C-1 form the amide to the two neighboring residues |
| homoLys (BCMA) | 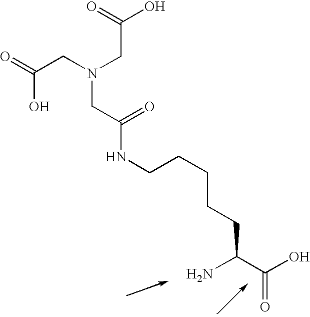 α-nitrogen and carboxy-carbon atom C-1 form the amide bonds to the two neighboring residues |
| homoSer | 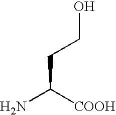 homo-serine |
| Hyp | 4-hydroxyproline |
| Ile | Isoleucine |
| Leu | Leucine |
| Lys | Lysine |
| Lys (biscarboxymethyl) | 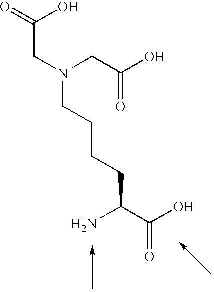 α-nitrogen and carboxy-carbon atom C-1 form the amide bonds to the two neighboring residues |
| Lys (BCMA) | 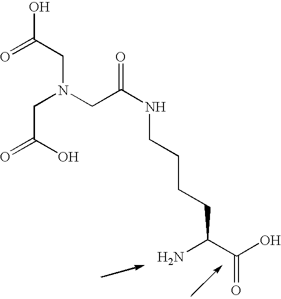 α-nitrogen and carboxy-carbon atom C-1 form the amide bonds to the two neighboring residues |
| Met | Methionine |
| Met (O) | 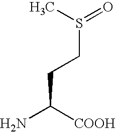 |
| Met (O₂) | 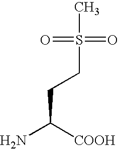 |
| 2-Nal | 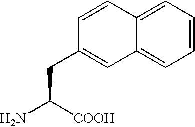 |
| Nle | 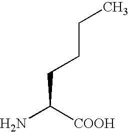 norleucine |
| Orn | Ornithine |
| Orn (biscarboxymethyl) | 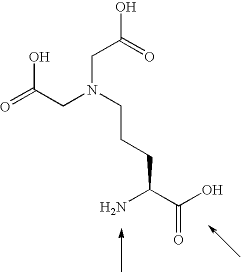 α-nitrogen and carboxy-carbon atom C-1 form the amide bond to the two neighboring residues |
| Orn (BCMA) | 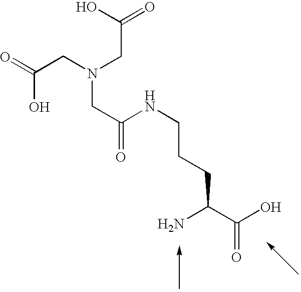 α-nitrogen and carboxy-carbon atom C-1 form the amide to the two neighboring residues |
| Phe | Phenylalanine |
| Pip | 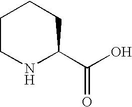 L-pipecolic acid |
| Pro | Proline |
| 2-PyAla | 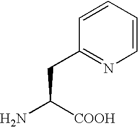 |
| 3-PyAla | 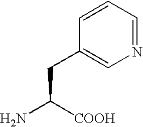 |
| 4-PyAla | 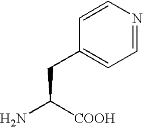 |
| Ser | Serine |
| tBuGly | 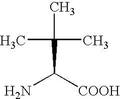 tert-butylglycine |
| Thr | Threonine |
| (4-thiazolyl) Ala | 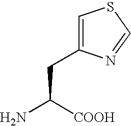 |
| Tic | 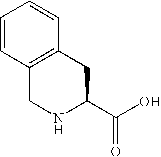 |
| Tyr | Tyrosine |
| Trp | Tryptophan |
| Val | Valine |

Amino acid abbreviations beginning with D- followed by a three letter code, such as D-Ser, D-His and so on, refer to the D-enantiomer of the corresponding amino acid, for example D-serine, D-histidine and so on.

### Pharmaceutical Compositions

As already mentioned, one aspect of the present invention provides a pharmaceutical composition (formulation) comprising a compound of the present invention. Appropriate embodiments of such formulations will often contain a compound of the invention in a concentration of from 10⁻³ mg/ml to 200 mg/ml, such as, e.g., from 10⁻¹ mg/ml to 100 mg/ml. The pH in such a formulation of the invention will typically be in the range of 2.0 to 10.0. The formulation may further comprise a buffer system, preservative(s), tonicity agent(s), chelating agent(s), stabilizer(s) and/or surfactant(s). In one embodiment of the invention the pharmaceutical formulation is an aqueous formulation, i.e. formulation comprising water, and the term “aqueous formulation” in the present context may normally be taken to indicate a formulation comprising at least 50% by weight (w/w) of water. Such a formulation is typically a solution or a suspension. An aqueous formulation of the invention in the form of an aqueous solution will normally comprise at least 50% (w/w) of water. Likewise, an aqueous formulation of the invention in the form of an aqueous suspension will normally comprise at least 50% (w/w) of water.

In another embodiment, a pharmaceutical composition (formulation) of the invention may be a freeze-dried (i.e. lyophilized) formulation intended for reconstitution by the physician or the patient via addition of solvents and/or diluents prior to use.

In a further embodiment, a pharmaceutical composition (formulation) of the invention may be a dried formulation (e.g. freeze-dried or spray-dried) ready for use without any prior dissolution.

In a further aspect, the invention relates to a pharmaceutical composition (formulation) comprising an aqueous solution of a compound of the present invention, and a buffer, wherein the compound of the invention is present in a concentration of 0.1-100 mg/ml or above, and wherein the formulation has a pH from about 2.0 to about 10.0.

In another embodiment of the invention, the pH of the formulation has a value selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0.

In a further embodiment, the buffer in a buffered pharmaceutical composition of the invention may comprise one or more buffer substances selected from the group consisting of sodium acetate, sodium carbonate, citrates, glycylglycine, histidine, glycine, lysine, arginine, sodium dihydrogen phosphate, disodium hydrogen phosphate, sodium phosphate, tris(hydroxymethyl)aminomethane (TRIS), bicine, tricine, malic acid, succinates, maleic acid, fumaric acid, tartaric acid and aspartic acid. Each one of these specific buffers constitutes an alternative embodiment of the invention.

In another embodiment, a pharmaceutical composition of the invention may comprise a pharmaceutically acceptable preservative, e.g. one or more preservatives selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, thiomerosal, bronopol, benzoic acid, imidurea, chlorohexidine, sodium dehydroacetate, chlorocresol, ethyl p-hydroxybenzoate, benzethonium chloride and chlorphenesine (3p-chlorphenoxypropane-1,2-diol). Each one of these specific preservatives constitutes an alternative embodiment of the invention. In a further embodiment of the invention the preservative is present in a concentration from 0.1 mg/ml to 20 mg/ml. In still further embodiments of such a pharmaceutical composition of the invention, the preservative is present in a concentration in the range of 0.1 mg/ml to 5 mg/ml, a concentration in the range of 5 mg/ml to 10 mg/ml, or a concentration in the range of 10 mg/ml to 20 mg/ml. The use of a preservative in pharmaceutical compositions is well known to the skilled person. For convenience, reference is made in this respect to Remington: The Science and Practice of Pharmacy, 20^{th} edition, 2000.

In a further embodiment of the invention the formulation further comprises a tonicity-adjusting agent, i.e. a substance added for the purpose of adjusting the tonicity (osmotic pressure) of a liquid formulation (notably an aqueous formulation) or a reconstituted freeze-dried formulation of the invention to a desired level, normally such that the resulting, final liquid formulation is isotonic or substantially isotonic. Suitable tonicity-adjusting agents may be selected from the group consisting of salts (e.g. sodium chloride), sugars and sugar alcohols (e.g. mannitol), amino acids (e.g. glycine, histidine, arginine, lysine, isoleucine, aspartic acid, tryptophan or threonine), alditols [e.g. glycerol (glycerine), 1,2-propanediol (propyleneglycol), 1,3-propanediol or 1,3-butanediol], polyethyleneglycols (e.g. PEG 400) and mixtures thereof.

Any sugar, such as a mono-, di- or polysaccharide, or a water-soluble glucan, including for example fructose, glucose, mannose, sorbose, xylose, maltose, lactose, sucrose, trehalose, dextran, pullulan, dextrin, cyclodextrin, soluble starch, hydroxyethyl starch or carboxymethylcellulose-sodium, may be used; in one embodiment, sucrose may be employed. Sugar alcohols (polyols derived from mono-, di-, oligo- or polysaccharides) include, for example, mannitol, sorbitol, inositol, galactitol, dulcitol, xylitol, and arabitol. In one embodiment, the sugar alcohol employed is mannitol. Sugars or sugar alcohols mentioned above may be used individually or in combination. There is no fixed limit to the amount used, as long as the sugar or sugar alcohol is soluble in the liquid composition (formulation) and does not adversely effect the stabilizing effects achieved using the methods of the invention. In one embodiment, the concentration of sugar or sugar alcohol is between about 1 mg/ml and about 150 mg/ml.

In further embodiments, the tonicity-adjusting agent is present in a concentration of from 1 mg/ml to 50 mg/ml, such as from 1 mg/ml to 7 mg/ml, from 8 mg/ml to 24 mg/ml, or from 25 mg/ml to 50 mg/ml. A pharmaceutical composition of the invention containing any of the tonicity-adjusting agents specifically mentioned above constitutes an embodiment of the invention. The use of a tonicity-adjusting agent in pharmaceutical compositions is well known to the skilled person. For convenience, reference is made to Remington: The Science and Practice of Pharmacy, 20^{th} edition, 2000.

In a still further embodiment of a pharmaceutical composition (formulation) of the invention, the formulation further comprises a chelating agent. Suitable chelating agents may be selected, for example, from salts of ethylenediaminetetraacetic acid (EDTA), citric acid, and aspartic acid, and mixtures thereof. The concentration of chelating agent will suitably be in the range from 0.1 mg/ml to 5 mg/ml, such as from 0.1 mg/ml to 2 mg/ml or from 2 mg/ml to 5 mg/ml. A pharmaceutical composition of the invention containing any of the chelating agents specifically mentioned above constitutes an embodiment of the invention. The use of a chelating agent in pharmaceutical compositions is well known to the skilled person. For convenience, reference is made to Remington: The Science and Practice of Pharmacy, 20^{th} edition, 2000.

In another embodiment of a pharmaceutical composition (formulation) of the invention, the formulation further comprises a stabilizer. The use of a stabilizer in pharmaceutical compositions is well known to the skilled person. For convenience, reference is made to Remington: The Science and Practice of Pharmacy, 20^{th} edition, 2000.

More particularly, particularly useful compositions of the invention include stabilized liquid pharmaceutical compositions whose therapeutically active components include an oligo- or polypeptide that possibly exhibits aggregate formation during storage in liquid pharmaceutical formulations. By “aggregate formation” is meant the formation of oligomers, which may remain soluble, or large visible aggregates that precipitate from the solution, as the result of a physical interaction between the oligo- or polypeptide molecules. The term “during storage” I refers to the fact that a liquid pharmaceutical composition or formulation, once prepared, is not normally administered to a subject immediately. Rather, following preparation, it is packaged for storage, whether in a liquid form, in a frozen state, or in a dried form for later reconstitution into a liquid form or other form suitable for administration to a subject. By “dried form” is meant the product obtained when a liquid pharmaceutical composition or formulation is dried by freeze-drying (i.e., lyophilization; see, for example, Williams and Polli (1984) J. Parenteral Sci. Technol. 38: 48-59), by spray-drying [see, e.g., Masters (1991) in Spray-Drying Handbook (5th edn.; Longman Scientific and Technical, Essex, U.K.), pp. 491-676; Broadhead et al. (1992) Drug Devel. Ind. Pharm. 18: 1169-1206; and Mumenthaler et al. (1994) Pharm. Res. 11: 12-20], or by air-drying [see, e.g., Carpenter and Crowe (1988) Cryobiology 25: 459-470; and Roser (1991) Biopharm. 4: 47-53]. Aggregate formation by an oligo- or polypeptide during storage of a liquid pharmaceutical composition can adversely affect biological activity of that peptide, resulting in loss of therapeutic efficacy of the pharmaceutical composition. Furthermore, aggregate formation may cause other problems, such as blockage of tubing, membranes or pumps when the oligo- or polypeptide-containing pharmaceutical composition is administered using an infusion system.

A pharmaceutical composition of the invention may further comprise an amount of an amino acid base sufficient to decrease aggregate formation by the oligo- or polypeptide during storage of the composition. By “amino acid base” is meant an amino acid, or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids for use in preparing a composition of the invention are those carrying a charged side chain, such as arginine, lysine, aspartic acid and glutamic acid. Any stereoisomer (i.e., L, D, or mixtures thereof) of a particular amino acid (e.g. methionine, histidine, arginine, lysine, isoleucine, aspartic acid, tryptophan or threonine, and mixtures thereof) or combinations of these stereoisomers, may be present in the pharmaceutical compositions of the invention so long as the particular amino acid is present either in its free base form or its salt form. In one embodiment, the L-stereoisomer of an amino acid is used. Compositions of the invention may also be formulated with analogues of these amino acids. By “amino acid analogue” is meant a derivative of a naturally occurring amino acid that brings about the desired effect of decreasing aggregate formation by the oligo- or polypeptide during storage of liquid pharmaceutical compositions of the invention. Suitable arginine analogues include, for example, aminoguanidine, ornithine and N-monoethyl-L-arginine. Suitable methionine analogues include ethionine and buthionine, and suitable cysteine analogues include S-methyl-L-cysteine. As with the amino acids per se, amino acid analogues are incorporated into compositions of the invention in either their free base form or their salt form. In a further embodiment of the invention, the amino acids or amino acid analogues are incorporated in a concentration which is sufficient to prevent or delay aggregation of the oligo- or polypeptide.

In a particular embodiment of the invention, methionine (or another sulfur-containing amino acid or amino acid analogue) may be incorporated in a composition of the invention to inhibit oxidation of methionine residues to methionine sulfoxide when the oligo- or polypeptide acting as the therapeutic agent is a peptide comprising at least one methionine residue susceptible to such oxidation. The term “inhibit” in this context refers to minimization of accumulation of methionine-oxidized species over time. Inhibition of methionine oxidation results in increased retention of the oligo- or polypeptide in its proper molecular form. Any stereoisomer of methionine (L or D) or combinations thereof can be used. The amount to be added should be an amount sufficient to inhibit oxidation of methionine residues such that the amount of methionine sulfoxide is acceptable to regulatory agencies. Typically, this means that no more than from about 10% to about 30% of forms of the oligo- or polypeptide wherein methionine is sulfoxidated are present. In general, this can be achieved by incorporating methionine in the composition such that the ratio of added methionine to methionine residues ranges from about 1:1 to about 1000:1, such as from about 10:1 to about 100:1.

In a further embodiment of the invention the formulation further comprises a stabilizer selected from high-molecular-weight polymers and low-molecular-weight compounds. Thus, for example, the stabilizer may be selected from substances such as polyethylene glycol (e.g. PEG 3350), polyvinyl alcohol (PVA), polyvinylpyrrolidone, carboxy-/hydroxycellulose and derivatives thereof (e.g. HPC, HPC-SL, HPC-L or HPMC), cyclodextrins, sulfur-containing substances such as monothioglycerol, thioglycolic acid and 2-methylthioethanol, and various salts (e.g. sodium chloride). A pharmaceutical composition of the invention containing any of the stabilizers specifically mentioned above constitutes an embodiment of the invention.

Pharmaceutical compositions of the present invention may also comprise additional stabilizing agents which further enhance stability of a therapeutically active oligo- or polypeptide therein. Stabilizing agents of particular interest in the context of the present invention include, but are not limited to: methionine and EDTA, which protect the peptide against methionine oxidation; and surfactants, notably nonionic surfactants which protect the polypeptide against aggregation or degradation associated with freeze-thawing or mechanical shearing.

Thus, in a further embodiment of the invention, the pharmaceutical formulation comprises a surfactant, particularly a nonionic surfactant. Examples thereof include ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (Tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl-serine, phosphatidyl-choline, phosphatidyl-ethanolamine, phosphatidyl-inositol, diphosphatidyl-glycerol and sphingomyelin), derivatives of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkyl ester and alkyl ether derivatives of lysophosphatidyl and phosphatidylcholines, e.g. lauroyl and myristoyl derivatives of lysophosphatidylcholine, dipalmitoylphosphatidylcholine, and modifications of the polar head group, i.e. cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (e.g. galactopyranoside), sphingoglycolipids (e.g. ceramides, gangliosides), dodecylphosphocholine, hen egg lysolecithin, fusidic acid derivatives (e.g. sodium tauro-dihydrofusidate, etc.), long-chain fatty acids (e.g. oleic acid or caprylic acid) and salts thereof, acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no. [577-11-7]), docusate calcium, CAS registry no. [128-49-4]), docusate potassium, CAS registry no. [749]-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulfonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (e.g. Tetronic's), which are tetrafunctional block copolymers derived from sequential addition of propylene oxide and ethylene oxide to ethylenediamine. The surfactant may also be selected from imidazoline derivatives and mixtures thereof. A pharmaceutical composition of the invention containing any of the surfactants specifically mentioned above constitutes an embodiment of the invention.

The use of a surfactant in pharmaceutical compositions is well-known to the skilled person. For convenience, reference is made to Remington: The Science and Practice of Pharmacy, 20^{th} edition, 2000.

Additional ingredients may also be present in a pharmaceutical composition (formulation) of the present invention. Such additional ingredients may include, for example, wetting agents, emulsifiers, antioxidants, bulking agents, metal ions, oleaginous vehicles, proteins (e.g. human serum albumin, gelatine or other proteins) and a zwitterionic species (e.g. an amino acid such as betaine, taurine, arginine, glycine, lysine or histidine). Such additional ingredients should, of course, not adversely affect the overall stability of the pharmaceutical formulation of the present invention.

Pharmaceutical compositions containing a compound according to the present invention may be administered to a patient in need of such treatment at several sites, for example at topical sites (e.g. skin and mucosal sites), at sites which bypass absorption (e.g. via administration in an artery, in a vein or in the heart), and at sites which involve absorption (e.g. in the skin, under the skin, in a muscle or in the abdomen).

Administration of pharmaceutical compositions according to the invention to patients in need thereof may be via several routes of administration. These include, for example, lingual, sublingual, buccal, in the mouth, oral, in the stomach and intestine, nasal, pulmonary (for example through the bronchioles and alveoli or a combination thereof), epidermal, dermal, transdermal, vaginal, rectal, ocular (for example through the conjunctiva), uretal and parenteral.

Compositions of the present invention may be administered in various dosage forms, for example in the form of solutions, suspensions, emulsions, microemulsions, multiple emulsion, foams, salves, pastes, plasters, ointments, tablets, coated tablets, rinses, capsules (e.g. hard gelatine capsules or soft gelatine capsules), suppositories, rectal capsules, drops, gels, sprays, powder, aerosols, inhalants, eye drops, ophthalmic ointments, ophthalmic rinses, vaginal pessaries, vaginal rings, vaginal ointments, injection solutions, in situ-transforming solutions (for example in situ gelling, in situ setting, in situ precipitating or in situ crystallizing), infusion solutions or implants.

Compositions of the invention may further be compounded in, or bound to, e,g. via covalent, hydrophobic or electrostatic interactions, a drug carrier, drug delivery system or advanced drug delivery system in order to further enhance the stability of the compound of the present invention, increase bioavailability, increase solubility, decrease adverse effects, achieve chronotherapy well known to those skilled in the art, and increase patient compliance, or any combination thereof. Examples of carriers, drug delivery systems and advanced drug delivery systems include, but are not limited to: polymers, for example cellulose and derivatives; polysaccharides, for example dextran and derivatives, starch and derivatives; poly(vinyl alcohol); acrylate and methacrylate polymers; polylactic and polyglycolic acid and block co-polymers thereof; polyethylene glycols; carrier proteins, for example albumin; gels, for example thermogelling systems, such as block co-polymeric systems well known to those skilled in the art; micelles; liposomes; microspheres; nanoparticulates; liquid crystals and dispersions thereof; L2 phase and dispersions thereof well known to those skilled in the art of phase behavior in lipid-water systems; polymeric micelles; multiple emulsions (self-emulsifying, self-microemulsifying); cyclodextrins and derivatives thereof; and dendrimers.

Compositions of the present invention are useful in the formulation of solids, semisolids, powders and solutions for pulmonary administration of a compound of the present invention, using, for example, a metered dose inhaler, dry powder inhaler or a nebulizer, all of which are devices well known to those skilled in the art.

Compositions of the present invention are useful in the formulation of controlled-release, sustained-release, protracted, retarded or slow-release drug delivery systems. Compositions of the invention are thus of value in the formulation of parenteral controlled-release and sustained-release systems well known to those skilled in the art (both types of systems leading to a many-fold reduction in the number of administrations required).

Of particular value are controlled-release and sustained-release systems for subcutaneous administration. Without limiting the scope of the invention, examples of useful controlled release systems and compositions are those containing hydrogels, oleaginous gels, liquid crystals, polymeric micelles, microspheres, nanoparticles,

Methods for producing controlled-release systems useful for compositions of the present invention include, but are not limited to, crystallization, condensation, co-crystallization, precipitation, co-precipitation, emulsification, dispersion, high-pressure homogenisation, encapsulation, spray-drying, microencapsulation, coacervation, phase separation, solvent evaporation to produce microspheres, extrusion and supercritical fluid processes. General reference is made in this context to Handbook of Pharmaceutical Controlled Release (Wise, D. L., ed. Marcel Dekker, New York, 2000), and Drugs and the Pharmaceutical Sciences, vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, for example a syringe in the form of a pen device. Alternatively, parenteral administration can be performed by means of an infusion pump. A further option is administration of a composition of the invention which is a liquid (typically aqueous) solution or suspension in the form of a nasal or pulmonary spray. As a still further option, a pharmaceutical composition of the invention can be adapted to transdermal administration (e.g. by needle-free injection or via a patch, such as an iontophoretic patch) or transmucosal (e.g. buccal) administration.

The term “stabilized formulation” refers to a formulation with increased physical stability, increased chemical stability or increased physical and chemical stability. The term “physical stability” in the context of a formulation containing an oligo- or polypeptide refers to the tendency of the peptide to form biologically inactive and/or insoluble aggregates as a result of exposure to thermo-mechanical stresses and/or interaction with interfaces and surfaces that are destabilizing, such as hydrophobic surfaces and interfaces. Physical stability of aqueous protein formulations is evaluated by means of visual inspection and/or turbidity measurements after exposing the formulation, filled in suitable containers (e.g. cartridges or vials), to mechanical/physical stress (e.g. agitation) at different temperatures for various time periods. Visual inspection of formulations is performed in a sharp focused light with a dark background. The turbidity of a formulation is characterized by a visual score ranking the degree of turbidity, for instance on a scale from 0 to 3 (in that a formulation showing no turbidity corresponds to a visual score 0, whilst a formulation showing visual turbidity in daylight corresponds to visual score 3). A formulation is normally classified physically unstable with respect to aggregation when it shows visual turbidity in daylight. Alternatively, the turbidity of a formulation can be evaluated by simple turbidity measurements well-known to the skilled person. Physical stability of aqueous oligo- or polypeptide formulations can also be evaluated by using a spectroscopic agent or probe of the conformational status of the peptide. The probe is preferably a small molecule that preferentially binds to a non-native conformer of the oligo- or polypeptide. One example of a small-molecular spectroscopic probe of this type is Thioflavin T. Thioflavin T is a fluorescent dye that has been widely used for the detection of amyloid fibrils. In the presence of fibrils, and possibly also other configurations, Thioflavin T gives rise to a new excitation maximum at about 450 nm, and enhanced emission at about 482 nm when bound to a fibril form. Unbound Thioflavin T is essentially non-fluorescent at the wavelengths in question.

Other small molecules can be used as probes of the changes in peptide structure from native to non-native states. Examples are the “hydrophobic patch” probes that bind preferentially to exposed hydrophobic patches of a polypeptide. The hydrophobic patches are generally buried within the tertiary structure of a polypeptide in its native state, but become exposed as it begins to unfold or denature. Examples of such small-molecular, spectroscopic probes are aromatic, hydrophobic dyes, such as anthracene, acridine, phenanthroline and the like. Other spectroscopic probes are metal complexes of amino acids, such as cobalt complexes of hydrophobic amino acids, e.g. phenylalanine, leucine, isoleucine, methionine, valine, or the like.

The term “chemical stability” of a pharmaceutical formulation as used herein refers to chemical covalent changes in oligo- or polypeptide structure leading to formation of chemical degradation products with potentially lower biological potency and/or potentially increased immunogenicity compared to the original molecule. Various chemical degradation products can be formed depending on the type and nature of the starting molecule and the environment to which it is exposed. Elimination of chemical degradation can most probably not be completely avoided and gradually increasing amounts of chemical degradation products may often be seen during storage and use of oligo- or polypeptide formulations, as is well known to the person skilled in the art. A commonly encountered degradation process is deamidation, a process in which the side-chain amide group in glutaminyl or asparaginyl residues is hydrolysed to form a free carboxylic acid. Other degradation pathways involve formation of higher molecular weight transformation products wherein two or more molecules of the starting substance are covalently bound to each other through transamidation and/or disulfide interactions, leading to formation of covalently bound dimer, oligomer or polymer degradation products (see, e.g., Stability of Protein Pharmaceuticals, Ahern. T. J. & Manning M. C., Plenum Press, New York 1992). Oxidation (of for instance methionine residues) may be mentioned as another variant of chemical degradation. The chemical stability of a formulation may be evaluated by measuring the amounts of chemical degradation products at various time-points after exposure to different environmental conditions (in that the formation of degradation products can often be accelerated by, e.g., increasing temperature). The amount of each individual degradation product is often determined by separation of the degradation products depending on molecule size and/or charge using various chromatographic techniques (e.g. SEC-HPLC and/or RP-HPLC).

Hence, as outlined above, a “stabilized formulation” refers to a formulation with increased physical stability, increased chemical stability, or increased physical and chemical stability. In general, a pharmaceutical composition (formulation) must be stable during use and storage (in compliance with recommended use and storage conditions) until the expiry date is reached.

A pharmaceutical composition (formulation) of the invention should preferably be stable for more than 2 weeks of usage and for more than two years of storage, more preferably for more than 4 weeks of usage and for more than two years of storage, desirably for more than 4 weeks of usage and for more than 3 years of storage, and most preferably for more than 6 weeks of usage and for more than 3 years of storage.

All references, including publications, patent applications and patents, cited herein are hereby incorporated by reference in their entirety and to the same extent as if each reference were individually and specifically indicated to be incorporated by reference and were set forth in its entirety herein (to the maximum extent permitted by law).

Headings and sub-headings are used herein for convenience only, and should not be construed as limiting the invention in any way.

The use of any and all examples, or exemplary language (including “for instance”, “for example”, “e.g.” and “such as”) in the present specification is intended merely to better illuminate the invention, and does not pose a limitation on the scope of the invention unless otherwise indicated. No language in the specification should be construed as indicating any non-claimed element as being essential to the practice of the invention.

The citation and incorporation of patent documents herein is done for convenience only, and does not reflect any view of the validity, patentability and/or enforceability of such patent documents.

The present invention includes all modifications and equivalents of the subject matter recited in the claims appended hereto, as permitted by applicable law.

### EXAMPLES

### List of Abbreviations Employed

AcOH acetic acid
BCMA [bis(carboxymethyl)amino]acetyl
Bn benzyl
BSA bovine serum albumin
DBU 1,8-diazabicyclo[5.4.0]undec-7-ene
DCM dichloromethane
Dde N-[1-(4,4-dimethyl-2,6-dioxocyclohex-1-ylidene)ethyl]
DIC diisopropylcarbodiimide
DIPEA ethyldiisopropylamine
DMAP 4-(dimethylamino)pyridine
DMF N,N-dimethylformamide
DMSO dimethylsulfoxide
EGTA glycol-bis(2-aminoethylether)-N,N,N′,N′-tetraacetic acid (ethyleneglycol tetraacetic acid)
FCS fetal calf serum
Fmoc 9H-fluoren-9-ylmethyloxycarbonyl
HBTU 2-(1H-benzotriazol-1-yl-)-1,1,3,3-tetramethyluronium hexafluorophosphate
HEPES 2-[4-(2-hydroxyethyl)-piperazin-1-yl]ethanesulfonic acid
HOAt 1-hydroxy-7-aza-benzotriazole
HOBt 1-hydroxybenzotriazole
HSA human serum albumin
IBMX 3-isobutyl-1-methylxanthine
MC1 melanocortin receptor subtype 1 (also denoted melanocortin receptor 1)
MC2 melanocortin receptor subtype 2 (also denoted melanocortin receptor 2)
MC3 melanocortin receptor subtype 3 (also denoted melanocortin receptor 3)
MC4 melanocortin receptor subtype 4 (also denoted melanocortin receptor 4)
MC5 melanocortin receptor subtype 5 (also denoted melanocortin receptor 5)
MeCN acetonitrile
MeOH methanol
min minutes
α-MSH α-form of melanocyte-stimulating hormone
MTX methotrexate
NEt₃ triethylamine
NMP N-methylpyrrolidin-2-one
OSu ester 2,5-dioxo-pyrrolidin-1-yl ester
PBS phosphate-buffered saline
PEI polyethyleneimine
PyBOP (benzotriazol-1-yloxy)trispyrrolidinophosphonium hexafluorophosphate
TFA trifluoroacetic acid
THF tetrahydrofuran
TSTU O-(N-succinimidyl)-N,N,N′,N′-tetramethyluronium tetrafluoroborate
UPLC ultra performance liquid chromatography

All compounds of the present invention can be synthesized by those skilled in the art using standard coupling and deprotection steps. Non-standard procedures and syntheses of special building blocks are described below. A description of necessary tools and synthetic methods including standard abbreviations for peptide synthesis can be found in “The Fine Art Of Solid Phase Synthesis”, 2002/3 Catalogue, Novabiochem.

### General Procedures

### Peptide Synthesis on an Applied Biosystems Peptide Synthesizer ABI-433A

The peptide is synthesized according to the Fmoc strategy on an Applied Biosystems 433 peptide synthesizer on a 0.25 mmol or 1.0 mmol scale using the manufacturer supplied FastMoc UV protocols which employ the Fmoc protected amino acid (4 equivalents), HOBt (4 equivalents), HBTU (4 equivalents) and DIPEA (8 equivalents) in NMP, and UV monitoring of the deprotection of the Fmoc protection group. Piperidine in NMP is used for deprotection of the Fmoc protected amino acids.

Cleavage from the Resin and Side-Chain Deprotection

After completed solid-phase peptide synthesis, the resin is extensively washed with DCM. The resin is then washed with a premixed solution of DCM-triisopropylsilane-water-mercaptoethanol (92.5:2.5:2.5:2.5). After filtration, a mixture of TFA-triisopropylsilane-water-mercaptoethanol (92.5:2.5:2.5:2.5; at least 40 ml per mmol of resin) is added, and the mixture agitated for 3 hours before the resin is drained and the filtrate is collected. The resin is washed with TFA-triisopropylsilane-water-mercaptoethanol (92.5:2.5:2.5:2.5) and the filtrate is collected. To the combined filtrates, ice-cold diethyl ether (10× the volume of the cleavage mixture) is added and the resulting precipitate is filtered off, washed with diethyl ether and dried.

### Purification and Quantification

The crude peptide is dissolved in a suitable mixture of water and MeCN or N-methylformamide and purified by reversed-phase preparative HPLC (Waters Deltaprep 4000 or Gilson) on a column containing C18-silica gel. Elution is performed with an increasing gradient of MeCN in water containing 0.1% TFA. Relevant fractions are checked by analytical HPLC or HPLC. Fractions containing the pure target peptide are mixed and concentrated under reduced pressure. The resulting solution is analyzed (HPLC, LCMS) and the product is quantified using a chemiluminescent nitrogen specific HPLC detector (Antek 8060 HPLC-CLND) or by measuring UV-absorption at 280 nm. The product is dispensed into glass vials. The vials are capped with Millipore glassfibre prefilters. Freeze-drying for three days affords the peptide trifluoroacetate as a white solid.

In the examples listed below, Rt values are retention times and the mass values are those detected by the mass spectroscopy (MS) detector and obtained using one of the following HPLC-MS or HPLC-MS devices (LCMS).

### LCMS (System 1)

Waters Micromass LCT Premier XE mass spectrometer; electrospray; m/z=100 to m/z=2000; step 0.1 amu; Waters Acquity UPLC BEH C₁₈, 1.7 μm, 2.1 mm×50 mm; water/acetonitrile containing 0.1% formic acid; gradient 5%→95% acetonitrile linear during 4.0 min; flow 0.4 ml/min.

### LCMS (System 2)

Sciex API-3000 Quadrupole MS, electrospray, m/z=300 to m/z=2000; column: Waters XTerra® MS C₁₈ 5 μm 3.0×50 mm; water/acetonitrile containing 0.05% TFA; gradient: 5%→90% acetonitrile from 0 to 7.5 min; flow 1.5 ml/min.

### LCMS (System 3)

Sciex API-100 Quadrupole MS, electrospray, m/z=300 to m/z=2000; column: Waters XTerra® MS C₁₈ 5 μm 3.0×50 mm; water/acetonitrile containing 0.05% TFA; gradient: 5%→90% acetonitrile from 0 to 7.5 min; flow 1.5 ml/min.

### Preparation of bis(tert-butoxycarbonylmethyl)aminoacetic acid

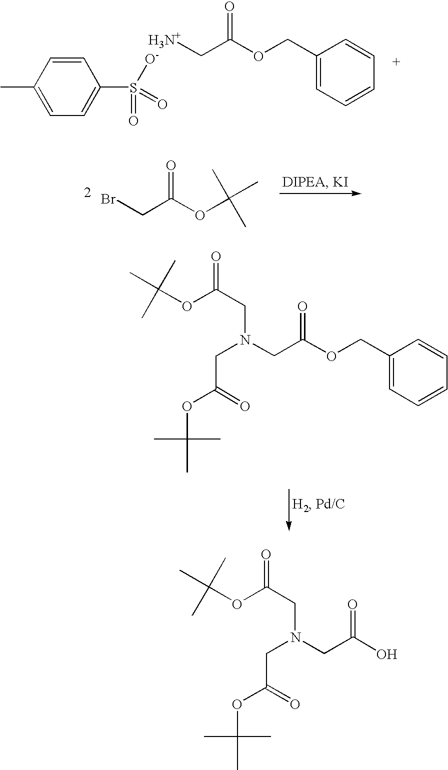

Bromoacetic acid tert-butyl ester (313.3 ml, 2.16 mol), DIPEA (179.5 ml, 1.08 mol) and potassium iodide (25.9 g, 216 mmol) were subsequently added to a solution of glycine benzyl ester p-methylbenzenesulfonic acid salt (72.95 g, 216 mmol) in N,N-dimethylformamide (730 ml). The resulting mixture was stirred at room temperature for 3 days under nitrogen. The solvent was evaporated in vacuo; the residue was diluted with dichloromethane (300 ml) and 5% aqueous solution of sodium carbonate (300 ml). The organic phase was washed with another portion of 5% aqueous solution of sodium carbonate (300 ml) and dried (Na₂SO₄). The solvent was evaporated in vacuo. The residue was filtered through silica gel (200 g, Fluka 60) using hexanes/ethylacetate mixture (2:1). After removal of solvent in vacuo the purification process was repeated twice. The solvent was evaporated to give bis(tert-butoxycarbonylmethyl)aminoacetic acid benzyl ester as a viscous yellow liquid.

Yield: 58.19 g (68%)

¹H NMR spectrum (300 MHz, CDCl₃): δ 7.49-7.38 (m, 5H); 5.15 (s, 2H); 3.69 (s, 2H); 3.54 (s, 4H); 1.44 (s, 18H).

Palladium on carbon (10%, 15 g) was added to a degassed solution of bis(tert-butoxycarbonylmethyl)aminoacetic acid benzyl ester (58.19 g, 148.8 mmol) in methanol (440 ml) and the reaction mixture was hydrogenated at 435 psi for 24 hrs. The mixture was filtered through a pad of Celite. The procedure was repeated three additional times. The filtrates were combined and evaporated in vacuo to give the title compound as a yellow solid. The residue was recrystallized four times from hexanes at −20° C. The solid was filtered off and dried in vacuo to give bis(tert-butoxycarbonylmethyl)aminoacetic acid.

Yield: 25.7 g (57%)

Melting point: 76-82° C.

¹H NMR spectrum (300 MHz, CDCl₃): δ 3.48 (s, 2H); 3.47 (s, 4H); 1.47 (s, 18H).

### Preparation of Fmoc-Lys(bis(tert-butoxycarbonylmethyl))-OH

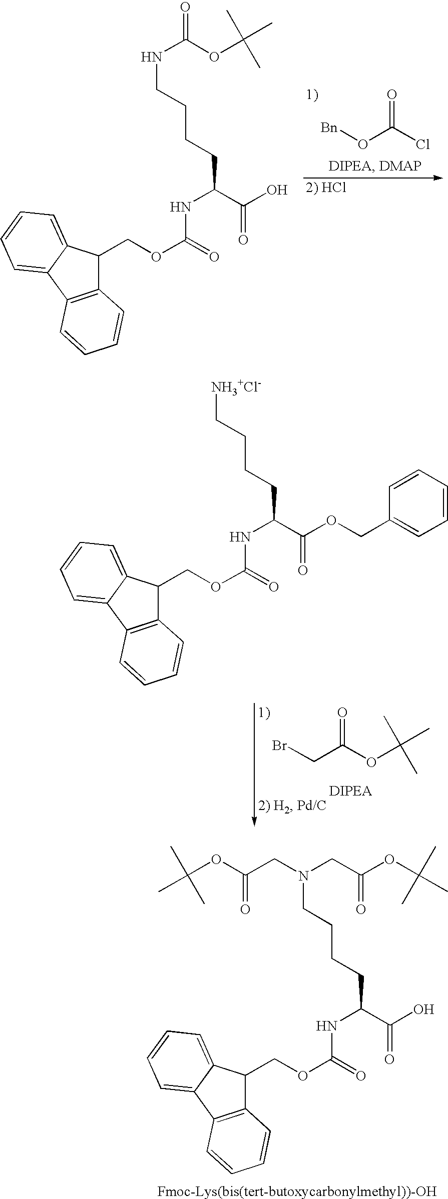

A solution of benzyl chloroformate (8.8 ml, 61.3 mmol) in DCM (50 mL) was added dropwise to a stirred solution of Fmoc-Lys(Boc)-OH (50 g, 53.6 mmol), DIPEA (27 ml, 78 mmol) and DMAP (650 mg, 5.3 mmol) in DCM (250 mL) at 0° C. The mixture was stirred at 0° C. for 24 hrs; then it was washed with 5% aqueous citric acid and water (200 mL). The organic layer was dried over anhydrous sodium sulfate and evaporated in vacuo. The residue was taken up in DCM (30 mL), filtered (S3) and purified by column chromatography (silica gel, hexanes/ethyl acetate 3:1). The fractions containing the product were evaporated in vacuo. The resulting solid was reevaporated from ethyl acetate to give Fmoc-Lys(Boc)-OBn as white amorphous powder.

Yield: 49.0 g (82%).

¹H NMR spectrum (300 MHz, CDCl₃): δ 7.79 (d, J=7.3 Hz, 2H); 7.62 (d, J=7.3 Hz, 2H); 7.48-7.29 (m, 9H); 5.44 (d, 1H); 5.21 (dd, 2H); 4.62-4.33 (m, 3H); 4.24 (t, 1H); 3.20-2.97 (m, 2H); 1.97-1.61 (m, 2H); 1.57-1.38 (m, 11H); 1.41-1.15 (m, 2H).

The above Fmoc-Lys(Boc)-OBn (31.32 g, 54 mmol) was dissolved in anhydrous DCM (60 mL), and solution of hydrogen chloride in dioxane (2.1 M, 205 mmol, 55 mL) was added. The reaction mixture was stirred at room temperature for 10 hrs before removal of the solvent under reduced pressure. The solid residue was dried on air. This crude product was used without further purification. LC/MS analysis proved a completion of the reaction.

The reaction was done in two batches.

Crude Fmoc-Lys-OBn HCl salt (50.8 g, 102 mmol) was dissolved in dry DMF (250 mL), and DIPEA (87 ml, 510 mmol), and tert-butyl bromoacetate (45 mL, 306 mmol) were added to the solution. The mixture was stirred at room temperature for 3 hrs, and DMF was removed under reduced pressure (at 50° C.). The residue was suspended in water (500 mL) and extracted with DCM (3×500 mL). The organic layer was dried over anhydrous sodium sulfate and evaporated in vacuo. The residue was purified by column chromatography (silica gel, gradient elution hexanes/ethyl acetate 9:1 to 7:3) to give Fmoc-Lys(bis(tert-butoxycarbonylmethyl))-OBn as pale yellow oil. Chromatography of mixed fractions was repeated.

Yield: 54.24 g (77%).

¹H NMR spectrum (300 MHz, CDCl₃): δ 7.76 (d, J=7.2 Hz, 2H); 7.60 (d, J=6.6 Hz, 2H); 7.45-7.23 (m, 9H); 5.51 (d, 2H); 5.17 (dd, 2H); 4.44-4.30 (m, 2H); 4.20-3.95 (m, 2H); 3.41 (s, 4H); 2.65-2.58 (m, 3H), 1.96-1.30 (m, 6H), 1.45 (s, 18H).

Fmoc-Lys(bis(tert-butoxycarbonylmethyl))-OBn (54.24 g, 79 mmol) was dissolved in methanol (500 mL). Palladium on carbon (5 wt %, 3.35 g) was added to the solution. The suspension was stirred under hydrogen atmosphere at room temperature. After 3 hrs, the mixture was filtered through Celite and the filtrate was concentrated. The crude product was purified by flash column chromatography (silica gel, DCM/methanol 95:5) to afford the title compound Fmoc-Lys(bis(tert-butoxycarbonylmethyl))-OH as white solid.

Yield: 31.4 g (67%).

Melting point: 51-52° C.

¹H NMR spectrum (300 MHz, CDCl₃): δ 7.76 (d, J=7.3 Hz, 2H); 7.60 (d, J=6.6 Hz, 2H); 7.39 (t, J=7.3 Hz, 2H); 7.30 (t, J=7.4 Hz, 2H); 5.67 (d, J=7.2 Hz, 1H); 4.31-4.53 (m, 3H); 4.17-4.26 (m, 1H); 3.54 (s, 1H); 2.64-2.91 (m, 2H); 1.44 (s, 18H), 1.19-1.99 (m, 6H).

### Preparation of (S)-2-Fmoc-amino-3-{2-[bis(tert-butoxycarbonylmethyl)amino]aetylamino}propionic acid

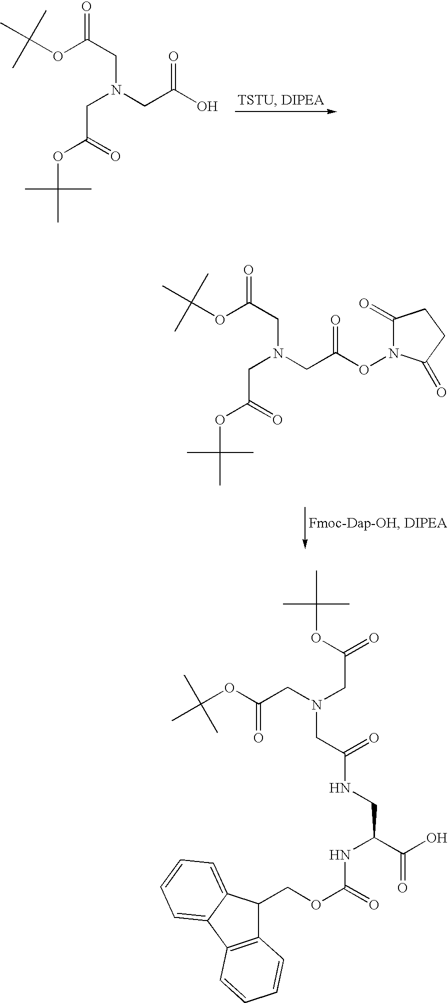

To bis(tert-butoxycarbonylmethyl)aminoacetic acid (1.0 g, 3.3 mmol) in dry THF (60 ml) was added DIPEA (0.84 ml, 4.9 mmol) and TSTU (1.78 g, 4.9 mmol) and the mixture was stirred for 3 days at room temperature. The solvent was removed in vacuo and the residue was divided by a mixture of ethylacetate (75 ml) and 5% citric acid in water (75 ml). The organic phase was dried over Na₂SO₄ and the solvent was removed in vacuo. The resulting crude bis(tert-butoxycarbonylmethyl)aminoacetic acid 2,5-dioxo-pyrrolidin-1-yl ester was pure enough for further synthesis.

To (S)-3-amino-2-(9H-fluoren-9-ylmethoxycarbonylamino)propionic acid (Fmoc-Dap-OH; 1.0 g, 3.06 mmol) in THF (50 mL) was added DIPEA (0.52 mL, 3.06 mmol) and bis(tert-butoxycarbonylmethyl)aminoacetic acid 2,5-dioxo-pyrrolidin-1-yl ester (2.43 g, 6.12 mmol) and the mixture was stirred for 3 hours before the solvent was removed in vacuo. The crude product was subjected to preparative HPLC to give 1.2 g (64% yield) of (S)-2-Fmoc-amino-3-{2-[bis(tert-butoxycarbonylmethyl)amino]acetylamino}propionic acid.

### Preparation of 16-(3-carboxy-propane-1-sulfonylamino)-16-oxo-hexadecanoic acid tert-butyl ester

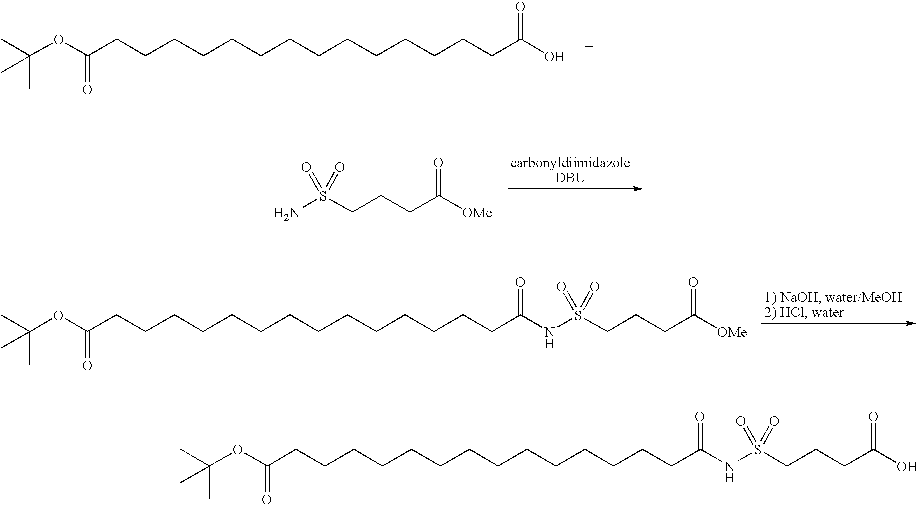

Hexadecanedioic acid mono-tert-butyl ester (5.14 g, 15.0 mmol) was dissolved in DCM (30 ml) and MeCN (30 ml). Carbonyldiimidazole (2.51 g, 15.45 mmol) was added and the mixture was stirred for 2 h. A solution of (4-sulfamoyl)butyric acid methyl ester (2.72 g, 15.0 mmol) in DCM (30 ml) was added, followed by addition of DBU (2.69 ml, 18 mmol). The mixture was stirred overnight and then concentrated under reduced pressure. The resulting residue was treated with 0.2 M aqueous citrate buffer pH 4.5 (preparation of the buffer: 0.2 mol of citric acid and 0.35 mol of NaOH dissolved in one liter of water). After 20 min, the resulting precipitate was collected by filtration and washed with water (150 ml).

This product was dissolved in MeOH (70 ml) and THF (20 ml). 1M aqueous NaOH (13 ml, 13 mmol) was slowly added and the mixture was stirred. After 40 min, a new portion of 1M aqueous NaOH (14.3 ml, 14.3 mmol) was slowly added. The mixture was stirred overnight and then poured into a mixture of water (150 ml) and 0.2 M aqueous citrate buffer pH 4.5 (150 ml). After 1 h, the resulting precipitate was collected by filtration, washed with water (100 ml) and dried to give the crude title compound. Recrystallization from acetone (300 ml) afforded 2.44 g (33% yield) of 16-(3-carboxy-propane-1-sulfonylamino)-16-oxo-hexadecanoic acid tert-butyl ester.

¹H NMR (DMSO-d6) δ 1.23 (m, 20H), 1.39 (s, 9H), 1.48 (m, 4H), 1.84 (m, 2H), 2.16 (t, J 7 Hz, 2H), 2.24 (t, J 7 Hz, 2H), 2.38 (t, J 7 Hz, 2H), 3.37 (m, partially overlapping with water peak at 3.33 ppm).

A typical example of a synthesis procedure which includes a cyclization step is as follows:

### Example 1

### (2-{2-[16-(Tetrazol-5-yl)hexadecanoylamino]ethoxy}ethoxy)acetyl-Gly-Ser-Gln-His-Lys(BCMA)-Nle-c[Glu-Hyp-D-Phe-Arg-Trp-Lys]-NH₂

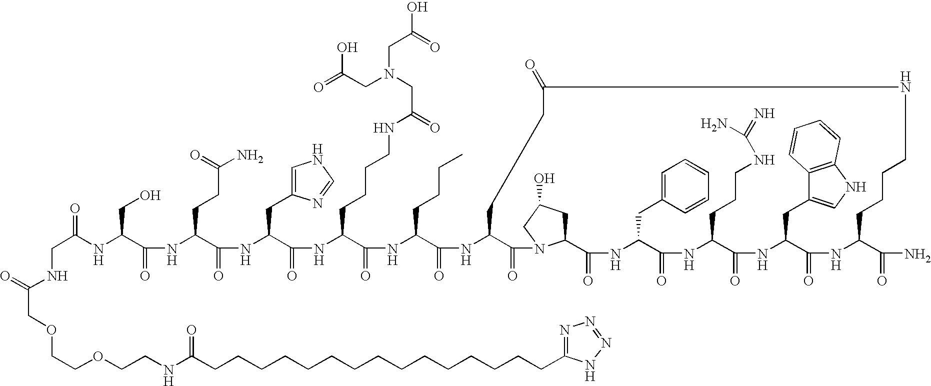

### Step A for Example 1

### Protected Peptide Resin Fmoc-c[Glu-Hyp(tBu)-D-Phe-Arg(Pbf)-Trp(Boc)-Lys]-NH-Rink Linker-Polystyrene

The synthesis was performed by using a MultiSynthTech synthesizer. Fmoc-Rink amide AM resin (10.56 g, 7.5 mmol; 4-(2′,4′-dimethoxyphenyl-Fmoc-aminomethyl)-phenoxyacetamidonorleucylaminomethylpolystyrene resin; 200-400 mesh; 0.71 mmol/g; Novabiochem 01-64-0038) was charged in a sintered glass reactor and swelled in NMP (105 ml). The resin was drained after 5 min.

### Removal of Fmoc

The resin was treated with a solution of 20% piperidine in NMP (105 ml) for 3 min. The resin was drained and the procedure was repeated twice. The resin was washed 6× with NMP (105 ml).

Acylation with Fmoc-Lys(Mtt)-OH.

In a separate flask, to Fmoc-Lys(Mtt)-OH (14.06 g, 22.5 mmol) in NMP (30 ml) and DCM (52.5 ml) was added a solution of HOBt (1M in NMP, 22.5 ml), before DIC (3.48 ml, 22.5 mmol) was added dropwise. After 20 min the solution was added to the resin and the mixture was agitated for 20 min before DIPEA (7.97 ml, 45 mmol) was added. The mixture was agitated for 100 min before the resin was drained and washed 4× with NMP (105 ml).

Acylation with Fmoc-Trp(Boc)-OH

The Fmoc group was removed as described above.

In a separate flask, to Fmoc-Trp(Boc)-OH (11.85 g, 22.5 mmol) in NMP (30 ml) and DCM (52.5 ml) was added a solution of HOBt (1M in NMP, 22.5 ml) before DIC (3.48 ml, 22.5 mmol) was added dropwise. After 20 min the solution was added to the resin and the mixture was agitated for 20 min before DIPEA (7.97 ml, 45 mmol) was added. The mixture was agitated for 100 min before the resin was drained and washed 4× with NMP (105 ml).

Using a similar procedure, the following amino acids were successively attached to the resin: Fmoc-Arg(Pbf)-OH, Fmoc-D-Phe-OH, Fmoc-Hyp(tBu)-OH, and Fmoc-Glu(2-phenylisopropyloxy)-OH.

### Selective Side-Chain Deprotection of Lys and Glu

The resin was shaken with a solution of 2% TFA and 3% triisopropylsilane in DCM (110 ml) for 10 min and drained. The procedure was repeated 6 times. The resin was washed with 4×DCM (105 ml), 2×10% DIPEA in DCM (105 ml) and 6×DCM (105 ml).

Side-Chain Cyclisation of Lys with Glu

In a separate flask, to PyBOB (11.71 g, 22.5 mmol) in NMP (42 ml) and DCM (57 ml) was added a solution of HOBt (1M in NMP, 22.5 ml). This mixture was added to the resin, followed by DIPEA (7.71 ml, 45 mmol) and the mixture was agitated for 16 hours. The resin was drained and washed 4× with NMP (105 ml) and 10×DCM (105 ml), and dried in vacuo.

### Step B for Example 1

### Automated Peptide Synthesis

The protected peptide resin Fmoc-c[Glu-Hyp(tBu)-D-Phe-Arg(Pbf)-Trp(Boc)-Lys]-NH-Rink AM linker-polystyrene obtained by step A (0.25 mmol) was charged in a reaction vessel on an ABI-433A peptide synthesis system, and the following acids were successively attached to the resin: Fmoc-Nle-OH, Fmoc-Lys(Dde)-OH, Fmoc-His(Trt)-OH, Fmoc-Gln(Trt)-OH, Fmoc-Ser(tBu)-OH, Fmoc-Gly-OH, Fmoc-8-amino-3,6-dioxaoctanoic acid and 16-(tetrazol-5-yl)hexadecanoic acid (available by the synthetic procedure described in WO 2007/009894).

### Step C for Example 1

### Solid-Phase Acylation at Lys Side Chain and Isolation of The Product

The resin was subsequently treated with hydrazine hydrate (2% in DMF, 3×3 min) before the resin was washed with NMP (5×).

In another flask, to bis(tert-butoxycarbonylmethyl)aminoacetic acid (379 mg, 1.25 mmol; available by the synthetic procedure described above) in NMP (3 ml) was added TSTU (376 mg, 1.25 mmol) and DIPEA (214 μL, 1.25 mmol). The mixture was stirred for 1 hour before it was transferred to the resin. The reaction mixture was agitated for 3 hours. The mixture was filtered and the resin was washed with NMP (5×) and DCM (6×). The product was cleaved from the resin and purified as described under general procedures to give the peptide trifluoroacetate as a white solid. Based on a nitrogen-specific HPLC detector (see above), the obtained yield of product was 0.0337 mmol (13%) corresponding to 72 mg of the TFA-free peptide.

LCMS (system 1): Rt=2.16 min; ((M+2)/2)=1068.0

### Example 2

### (2-{2-[16-(Tetrazol-5-yl)hexadecanoylamino]ethoxy}ethoxy)acetyl-Gly-Ser-Gln-His-β-Dap(BCMA)-Nle-c[Glu-Hyp-D-Phe-Arg-Trp-Lys]-NH₂

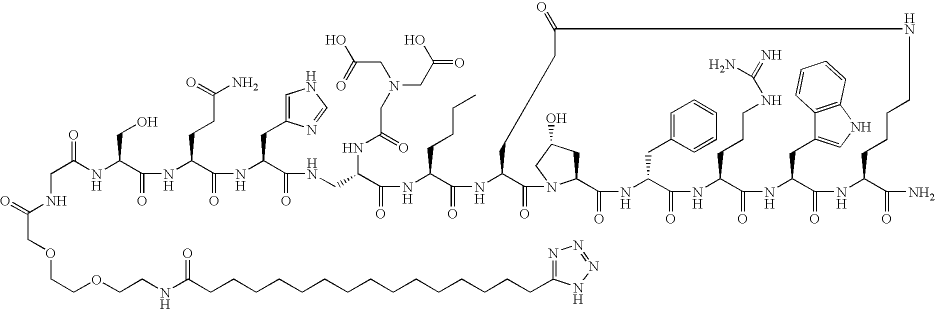

Peptide [2-{2-[16-(tetrazol-5-yl)hexadecanoylamino]ethoxy}ethoxy]acetyl-Gly-Ser-Gln-His-β-Dap-Nle-c[Glu-Hyp-D-Phe-Arg-Trp-Lys]-NH₂ was prepared similarly to the procedures of step A and step B described for Example 1. Boc-Dap(Fmoc)-OH was used for introducing the β-Dap residue. Cleavage from the resin, ether precipitation and purification were done similarly to the general procedures described above. N-Acylation at the free nitrogen atom of the β-Dap residue was then performed in the following manner.

### Solution-Phase N-Acylation and Removal of Tert-Butyl Groups

In a small test tube, bis(tert-butoxycarbonylmethyl)aminoacetic acid (20 mg, 0.065 mmol) and TSTU (20 mg, 0.065 mmol) were mixed with NMP (0.6 ml). DIPEA (0.027 ml, 0.156 mmol) was added to give a yellowish solution. The tube was capped and shaken for 2 h. The resulting yellow OSu ester solution was then used for the acylation described below.

In a test tube, the TFA salt of peptide [2-{2-[16-(tetrazol-5-yl)hexadecanoylamino]ethoxy}ethoxy]acetyl-Gly-Ser-Gln-His-β-Dap-Nle-c[Glu-Hyp-D-Phe-Arg-Trp-Lys]-NH₂ (0.026 mmol) was dissolved in NMP (1.2 ml). DIPEA (0.029 ml, 0.169 mmol) was added. To the resulting clear colourless solution, the OSu ester solution (0.6 ml) was added. The tube was capped and shaken. LCMS indicated completed reaction after 3 h. After being shaken for 22 h, the reaction mixture was dropped into diethylether (40 ml). The resulting precipitate was collected by centrifugation and washed again with diethylether (40 ml). The liquid phase was removed by centrifugation. This afforded a sticky white-yellowish residue. A premixed solution of triisopropylsilane (0.5 ml) and ethandithiol (0.5 ml) in TFA (9 ml) was added to the sticky residue. The resulting clear colourless solution was stirred for 80 min and then concentrated to give a liquid residue (appr. 2 ml). The liquid was treated with diethylether (40 ml) to give a white precipitate. The precipitate was collected by centrifugation, washed again with diethylether (40 ml) and dried to give a white solid. HPLC purification and freeze-drying afforded the target peptide as a white solid. The obtained yield of product TFA salt was corresponding to 23 mg (13%) of the salt-free peptide.

LCMS (system 1): Rt=2.03 min; ((m+2)/2)=1047.0

### Example 3

### (2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-Lys(biscarboxymethyl)-Nle-c[Glu-Hyp-D-Phe-Arg-Trp-Lys]-NH₂

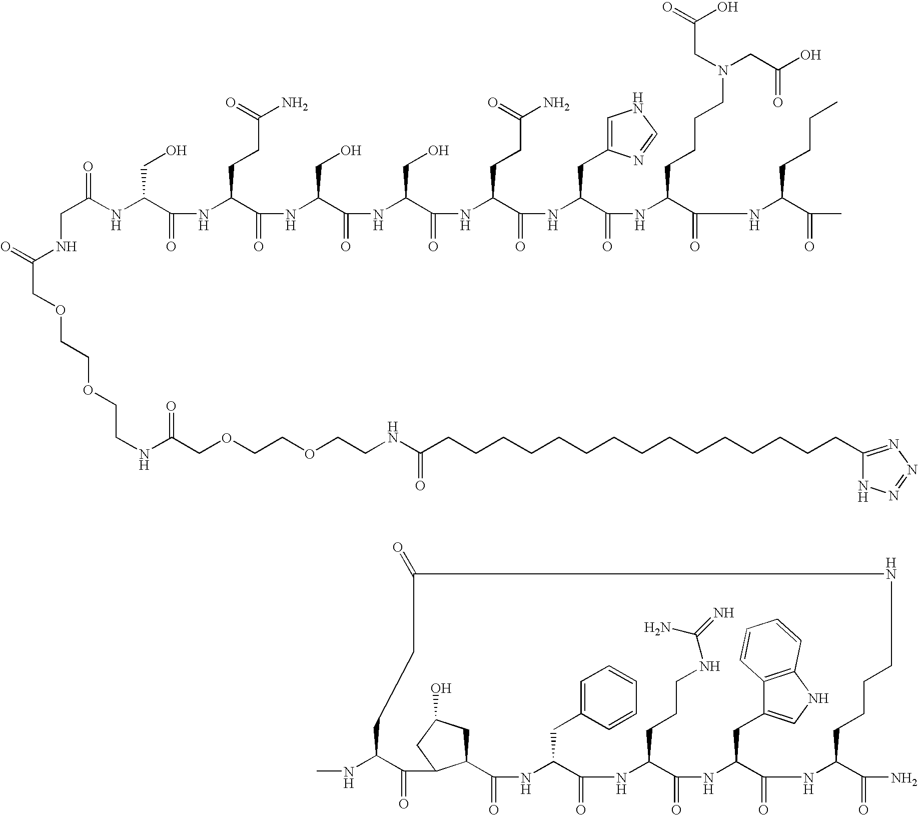

Peptide (2-{2-[2-(2-{2-[16-(tetrazol-5-yl)hexadecanoylamino]ethoxy}ethoxy)acetylamino]ethoxy}ethoxy)acetyl-Gly-D-Ser-Gln-Ser-Ser-Gln-His-Lys-Nle-c[Glu-Hyp-D-Phe-Arg-Trp-Lys]-NH₂ was prepared similarly to the procedures of step A and step B described for Example 1. Cleavage from the resin and ether precipitation was done similarly to the general procedures described above to give the crude peptide TFA salt. Reductive alkylation at the Lys side chain was then performed in the following manner.

Solution-Phase Reductive Dialkylation with Glyoxalic Acid

The crude peptide (from 0.25 mmol of Rink AM resin) was dissolved in a mixture of MeOH (8.5 ml), N-methylformamide (5 ml), water (3.4 ml) and 0.2 M citrate buffer pH 4.5 (4.5 ml, 0.9 mmol; preparation of the buffer: citric acid 0.2 M and NaOH 0.35 M). Glyoxalic acid monohydrate (0.212 g, 2.3 mmol) and a freshly prepared solution of sodium cyanoborohydride (0.057 g, 0.91 mmol) in MeOH (0.6 ml) were added. The mixture was stirred for approximately 24 h. LCMS indicated completed N,N-dialkylation. The mixture was concentrated under reduced pressure to give a liquid residue. This was diluted with water and acidified with TFA (0.25 ml). HPLC purification and freeze-drying afforded the target peptide as a white solid. The obtained yield of product TFA salt was corresponding to 50 mg (8%) of the salt-free peptide.

LCMS (system 2): Rt=2.14 min; ((m+2)/2)=1263.4

Alternatively, the Lys(biscarboxymethyl) residue can be introduced by using Fmoc-Lys(bis(tert-butoxycarbonylmethyl))-OH (available by the synthetic procedure described above).

### Example 4

### (2-{2-[16-(Tetrazol-5-yl)hexadecanoylamino]ethoxy}ethoxy)acetyl-Gly-Ser-Gln-Ser-Dap(BCMA)-Nle-c[Glu-Hyp-D-Phe-Arg-Trp-Lys]-NH₂

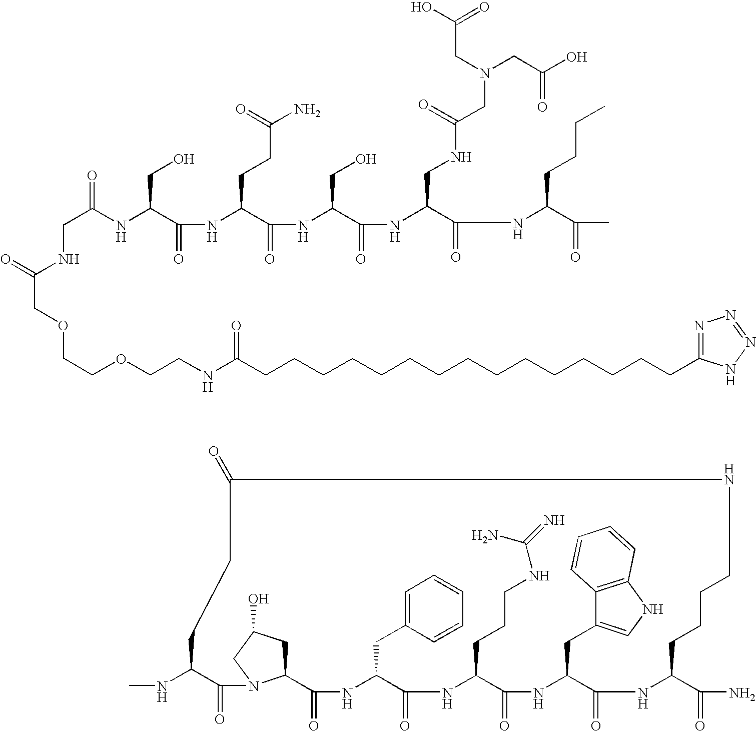

The compound was prepared similarly to the procedures of step A and step B described for Example 1. The Dap(BCMA) residue was introduced using (S)-2-Fmoc-amino-3-{2-[bis(tert-butoxycarbonylmethyl)amino]acetylamino}propionic acid (available by the synthetic procedure described above). The obtained yield of peptide TFA salt was corresponding to 56 mg (11%) of the salt-free peptide.

LCMS (system 1): Rt=2.29 min; ((m+2)/2)=1022.0

### Example 5

### (2-{2-[16-(Tetrazol-5-yl)hexadecanoylamino]ethoxy}ethoxy)acetyl-Gly-Ser-Gln-Ser-Lys(biscarboxymethyl)-Nle-c[Glu-Hyp-D-Phe-Arg-Trp-Lys]-NH₂

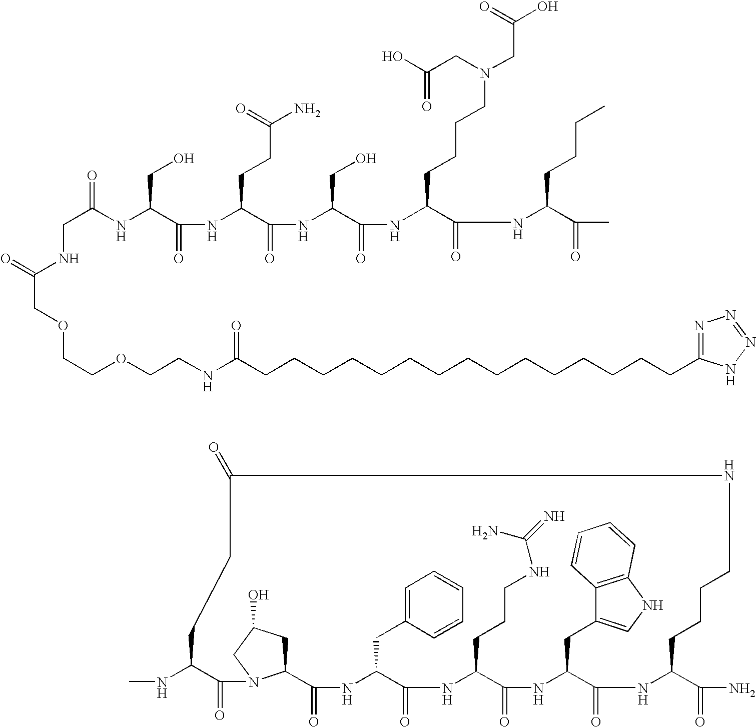

Protected peptide resin [2-{2-[16-(tetrazol-5-yl)hexadecanoylamino]ethoxy}ethoxy]acetyl-Gly-Ser(tBu)-Gln(Trt)-Ser(tBu)-Lys(Dde)-Nle-c[Glu-Hyp(tBu)-D-Phe-Arg(Pbf)-Trp(Boc)-Lys]-NH-Rink AM linker-polystyrene was prepared similarly to the procedures of step A and step B described for Example 1. The resin was subsequently treated with hydrazine hydrate (2% in DMF, 3×3 min) before the resin was washed with NMP (5×). Solid-phase reductive dialkylation at the Lys side chain was then performed in the following manner. The resin was treated for 16 h with a solution of glyoxalic acid (10 equivalents) and sodium cyanoborohydride (15 equivalents) in NMP/MeOH/acetic acid 7:3:1. Cleavage from the resin, purification and freeze-drying afforded the peptide as a white solid. The obtained yield of product TFA salt was corresponding to 45 mg (9%) of the salt-free peptide.

LCMS (system 1): Rt=2.24 min; ((m+2)/2)=1014.5

### Example 6

### (2-{2-[16-(Tetrazol-5-yl)hexadecanoylamino]ethoxy}ethoxy)acetyl-Gly-Ser-Gln-His-Dap(biscarboxymethyl)-Nle-c[Glu-Hyp-D-Phe-Arg-Trp-Lys]-NH₂

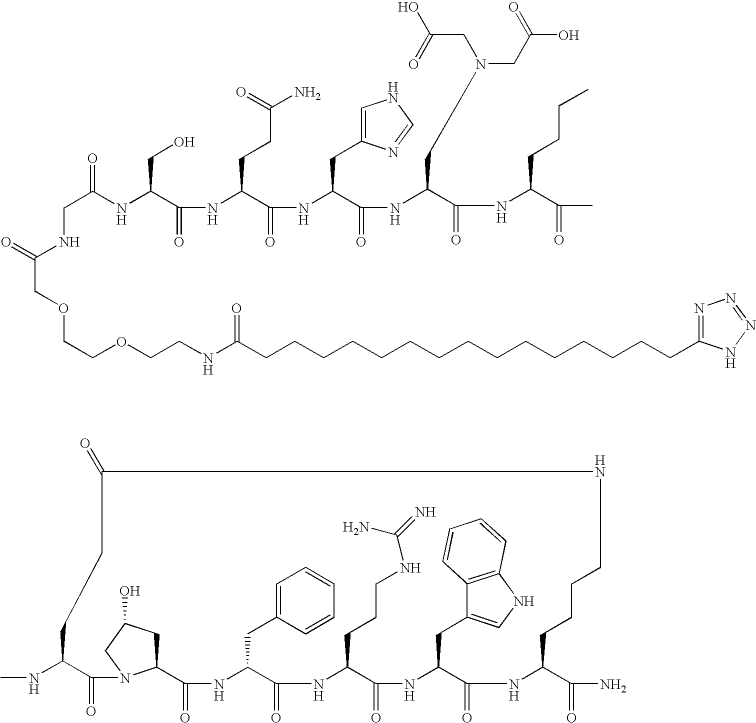

The compound was prepared similarly to the procedure described for Example 3.

LCMS (system 3): Rt=3.98 min; ((m+2)/2)=1018.5

### Example 7

### (2-{2-[16-(Tetrazol-5-yl)hexadecanoylamino]ethoxy}ethoxy)acetyl-Gly-Ser-Gln-His-Lys(biscarboxymethyl)-Nle-c[Glu-Hyp-D-Phe-Arg-Trp-Lys]-NH₂

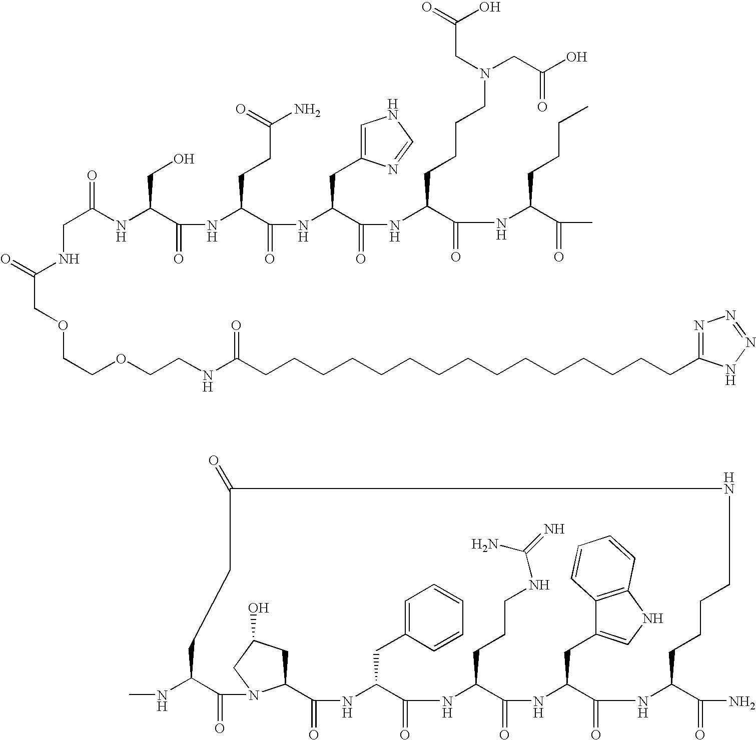

The compound was prepared similarly to the procedure described for Example 3.

LCMS (system 1): Rt=2.07 min; ((m+2)/2)=1039.5

### Example 8

### (2-{2-[16-(Tetrazol-5-yl)hexadecanoylamino]ethoxy}ethoxy)acetyl-Gly-Ser-Gln-His-Orn(BCMA)-Nle-c[Glu-Hyp-D-Phe-Arg-Trp-Lys]-NH₂

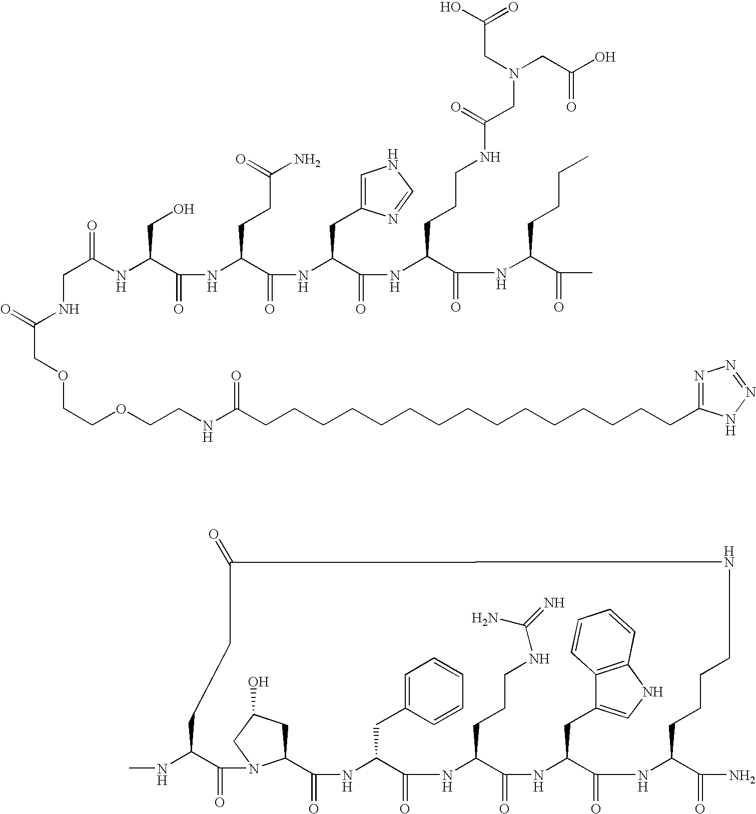

The compound was prepared similarly to the procedure described for Example 1.

LCMS (system 1): Rt=2.07 min; ((m+2)/2)=1061.0

### Example 9

### (2-{2-[16-(Tetrazol-5-yl)hexadecanoylamino]ethoxy}ethoxy)acetyl-Gly-Ser-Gln-His-Dap(BCMA)-Nle-c[Glu-Hyp-D-Phe-Arg-Trp-Lys]-NH₂

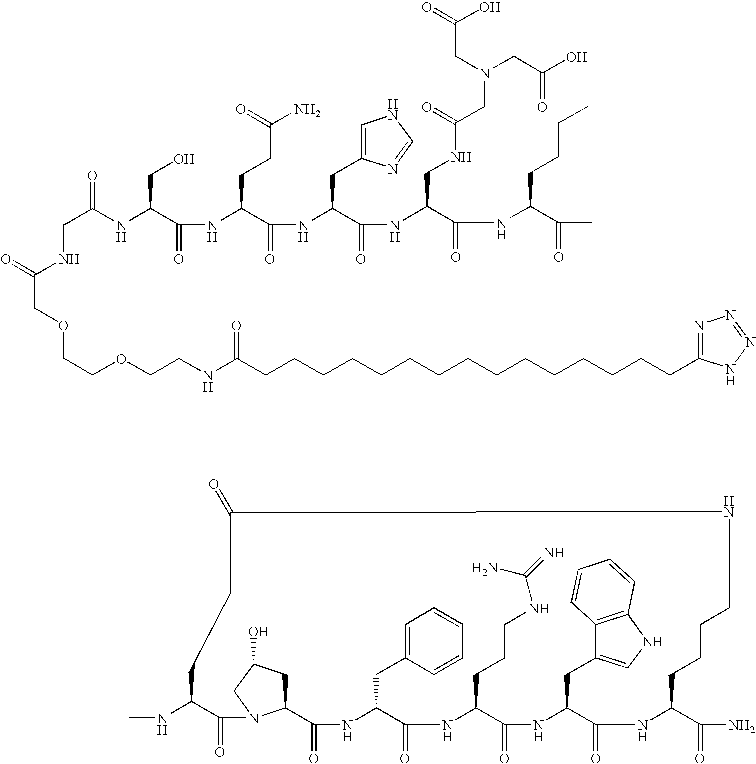

The compound was prepared similarly to the procedure described for Example 2.

LCMS (system 3): Rt=4.25 min; ((m+2)/2)=1047.3

### Example 10

### (2-{2-[16-(Tetrazol-5-yl)hexadecanoylamino]ethoxy}ethoxy)acetyl-Gly-Ser-Gln-His-Dab(BCMA)-Nle-c[Glu-Hyp-D-Phe-Arg-Trp-Lys]-NH₂

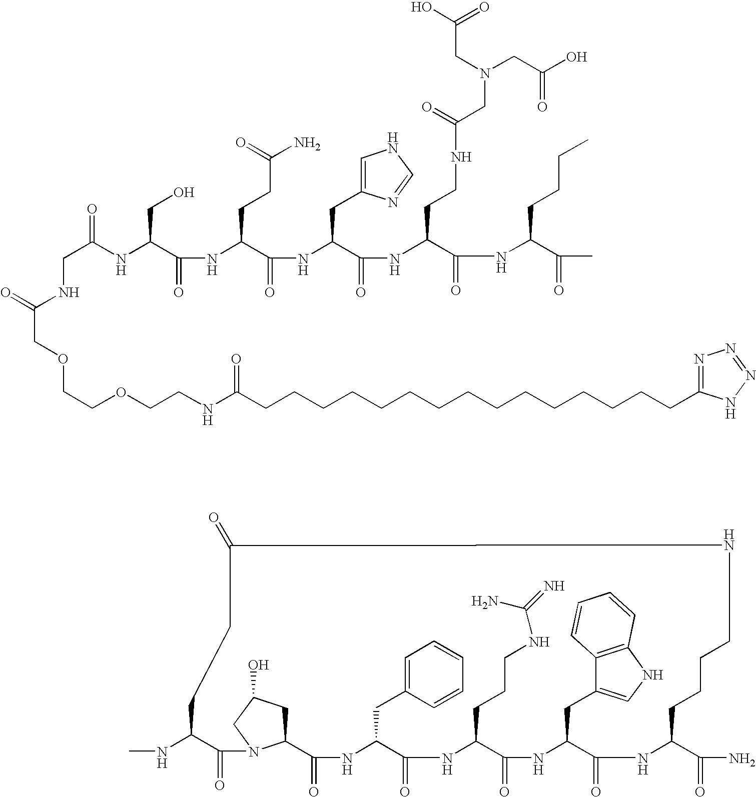

The compound was prepared similarly to the procedure described for Example 1.

LCMS (system 1): Rt=2.07 min; ((m+2)/2)=1054.0

### Example 11

### [2-(2-{4-[16-(Tetrazol-5-yl)hexadecanoylsulfamoyl]butanoylamino}ethoxy)ethoxy]acetyl-Gly-Ser-Gln-His-Dap(BCMA)-Nle-c[Glu-Hyp-D-Phe-Arg-Trp-Lys]-NH₂

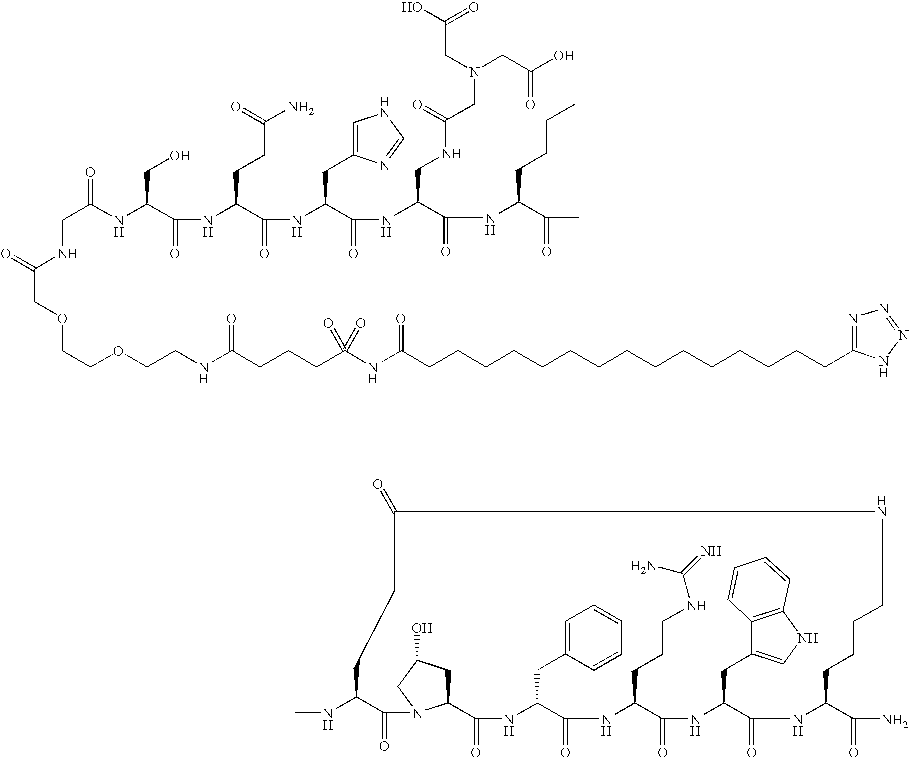

The compound was prepared similarly to the procedure described for Example 2 by using the building block 4-(N-(16-(tetrazol-5-yl)hexadecanoyl)sulfamoyl)butyric acid (available by the synthetic procedure described in WO 2007/009894).

LCMS (system 1): Rt=2.16 min; ((m+2)/2)=1121.5

### Example 12

### (2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-β-Ala-Lys(biscarboxymethyl)-Nle-c[Glu-Hyp-D-Phe-Arg-Trp-Lys]-NH₂

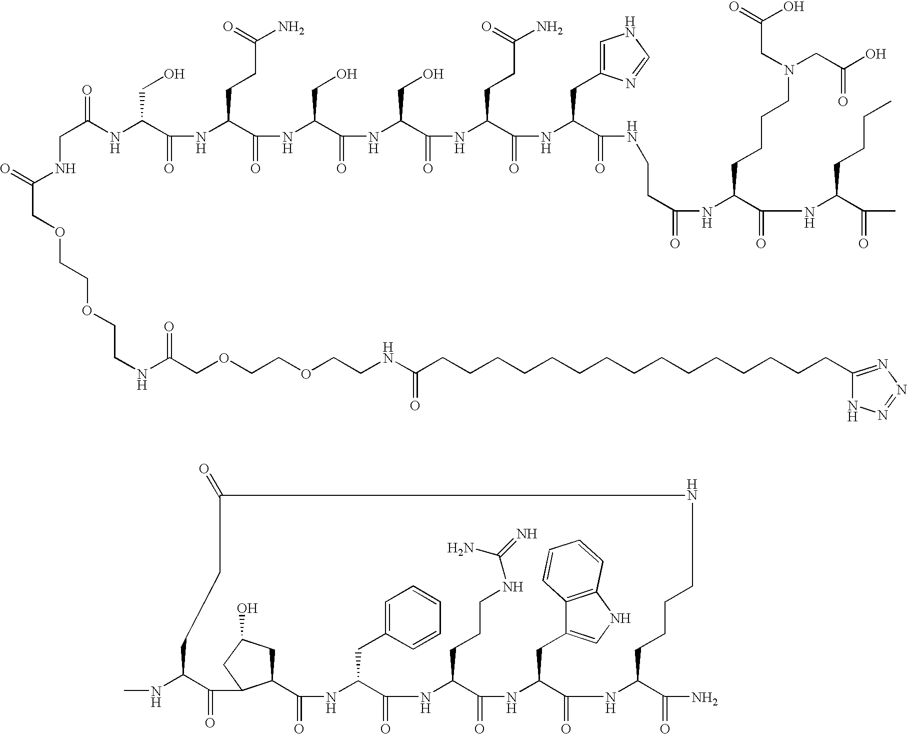

The compound was prepared similarly to the procedure described for Example 3.

LCMS (system 2): Rt=5.08 min; ((m+2)/2)=1299.3

### Example 13

### {2-[2-(15-Carboxypentadecanoylamino)ethoxy]ethoxy}acetyl-Gly-Ser-Gln-His-Dap(BCMA)-Nle-c[Glu-Hyp-D-Phe-Arg-Trp-Lys]-NH₂

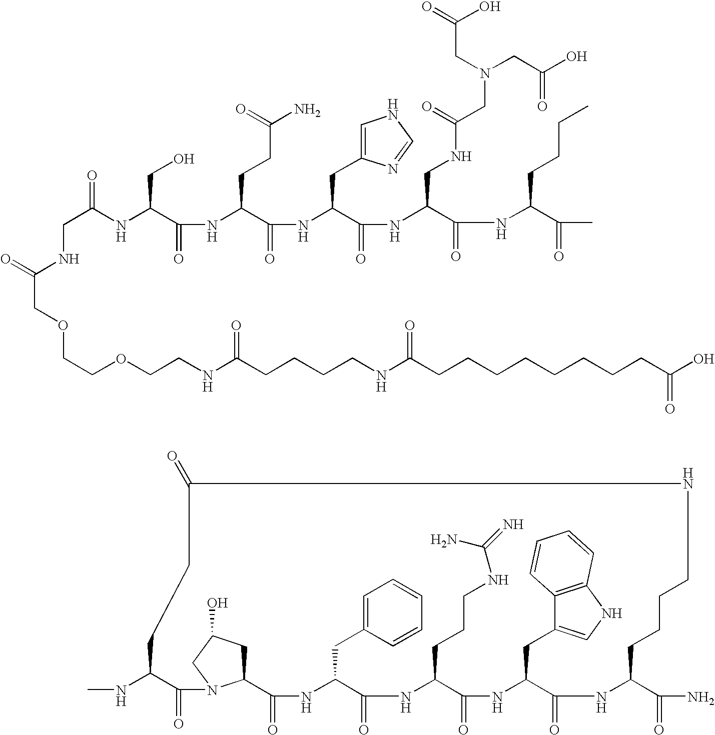

The compound was prepared similarly to the procedure described for Example 4 by using the building block hexadecanedioic acid mono-tert-butyl ester (available by the synthetic procedure described in: U. Widmer, Synthesis 1983, 135).

LCMS (system 1): Rt=2.07 min; ((m+2)/2)=1028.0

### Example 14

### (2-{2-[2-(2-{2-[2-(2-{2-[2-(2-{2-[16-(Tetrazol-5-yl)hexadecanoylamino]ethoxy}ethoxy)acetylamino]ethoxy}ethoxy)acetylamino]ethoxy}ethoxy)acetylamino]ethoxy}ethoxy)acetyl-Gly-Ser-Gln-Ser-Dap(BCMA)-Nle-c[Glu-Hyp-D-Phe-Arg-Trp-Lys]-NH₂

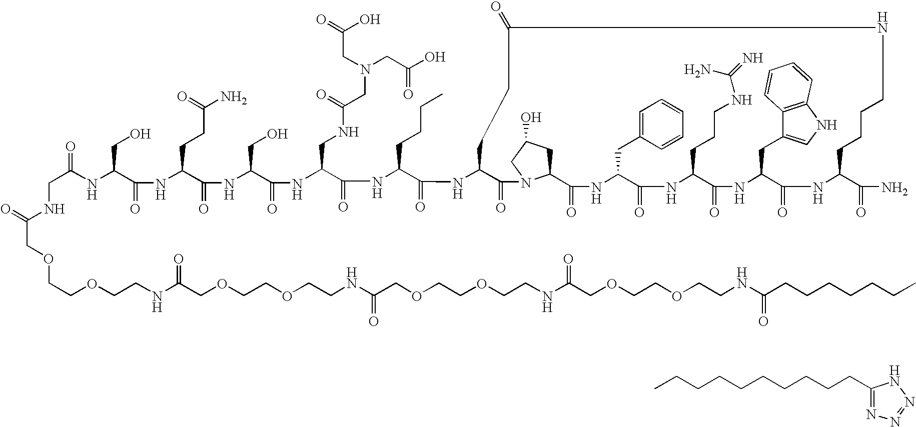

The compound was prepared similarly to the procedure described for Example 1.

LCMS (system 1): Rt=2.21 min; ((m+2)/2)=1239.6

### Example 15

### (2-{2-[16-(Tetrazol-5-yl)hexadecanoylamino]ethoxy}ethoxy)acetyl-Gly-Ser-Gln-Ser-Lys(BCMA)-Nle-c[Glu-Hyp-D-Phe-Arg-Trp-Lys]-NH₂

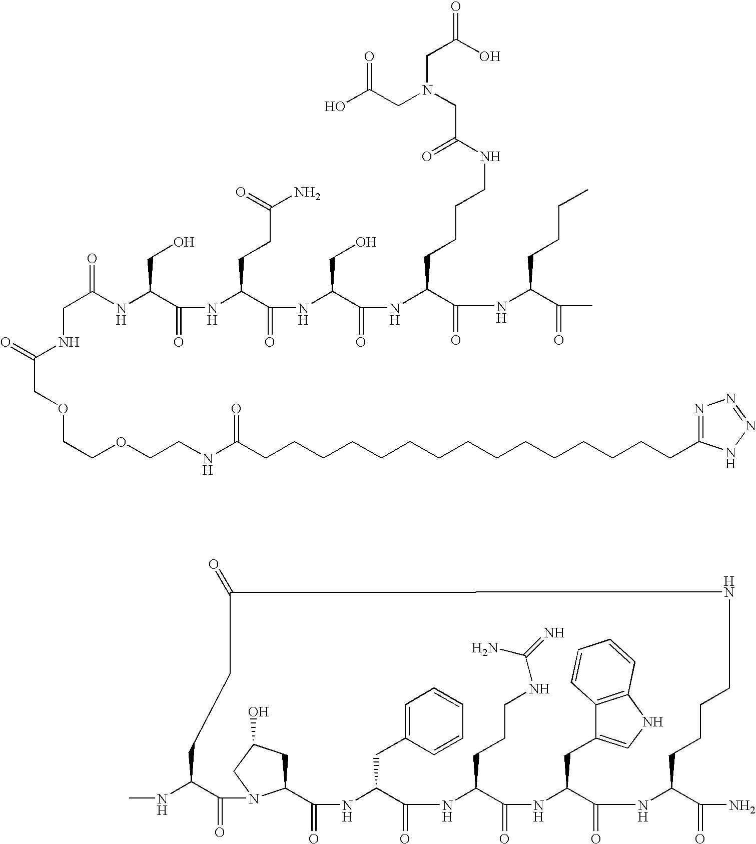

The compound was prepared similarly to the procedure described for Example 1.

LCMS (system 1): Rt=2.20 min; ((m+2)/2)=1043.0

### Example 16

### (2-{2-[2-(2-{2-[2-(2-{2-[2-(2-{2-[16-(Tetrazol-5-yl)hexadecanoylamino]ethoxy}ethoxy)acetylamino]ethoxy}ethoxy)acetylamino]ethoxy}ethoxy)acetylamino]ethoxy}ethoxy)acetyl-Gly-Ser-Gln-His-Dap(BCMA)-Nle-c[Glu-Hyp-D-Phe-Arg-Trp-Lys]-NH₂

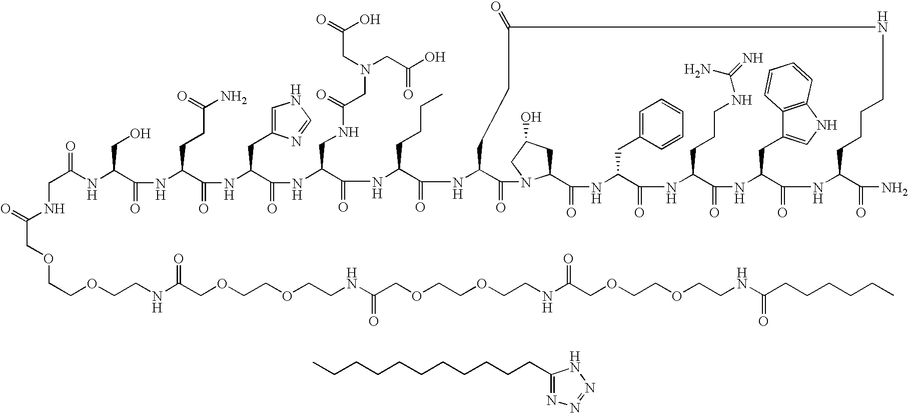

The compound was prepared similarly to the procedure described for Example 1.

LCMS (system 1): Rt=1.98 min; ((m+2)/2)=1264.7

### Example 17

### (2-{2-[16-(Tetrazol-5-yl)hexadecanoylamino]ethoxy}ethoxy)acetyl-Glu-Ser-Gln-His-Dap(BCMA)-Nle-c[Glu-Hyp-D-Phe-Arg-Trp-Lys]-NH₂

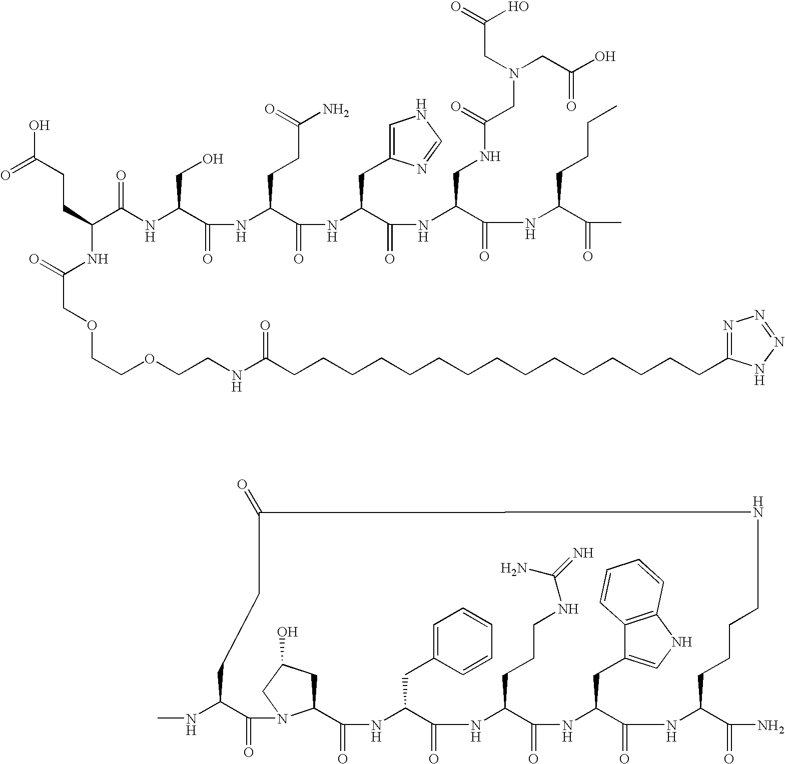

The compound was prepared similarly to the procedure described for Example 1.

LCMS (system 1): Rt=2.08 min; ((m+2)/2)=1083.0

### Example 18

### (2-{2-[2-(2-{2-[(S)-4-Carboxy-4-(17-carboxyheptadecanoylamino)butanoylamino]ethoxy}ethoxy)acetylamino]ethoxy}ethoxy)acetyl-Gly-Ser-Gln-His-Dap(BCMA)-Nle-c[Glu-Hyp-D-Phe-Arg-Trp-Lys]-NH₂

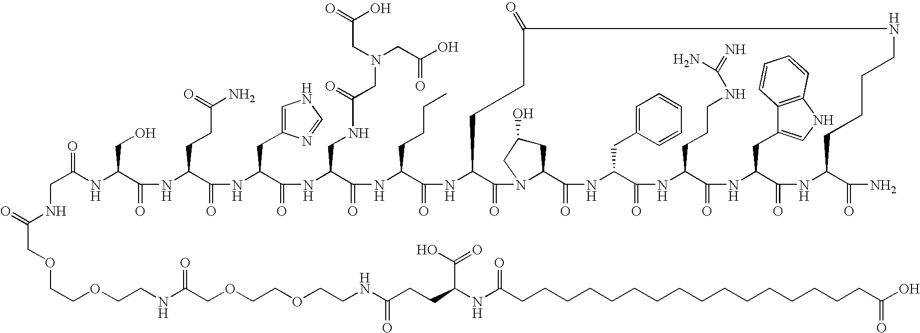

The compound was prepared similarly to the procedure described for Example 1 by using the building block octadecanedioic acid mono-tert-butyl ester (available by the synthetic procedure described in: U. Widmer, Synthesis 1983, 135).

LCMS (system 1): Rt=2.24 min; ((m+2)/2)=1179.1

### Example 19

### (2-{2-[16-(Tetrazol-5-yl)hexadecanoylamino]ethoxy}ethoxy)acetyl-Gly-Ser-Gln-Tyr-Dap(BCMA)-Nle-c[Glu-Hyp-D-Phe-Arg-Trp-Lys]-NH₂

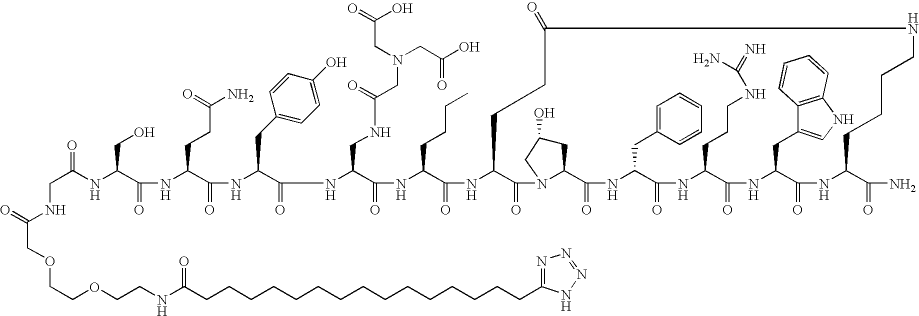

The compound was prepared similarly to the procedure described for Example 2.

LCMS (system 1): Rt=2.28 min; ((m+2)/2)=1060.0

### Example 20

### (2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-β-Ala-Lys(biscarboxymethyl)-Nle-c[Glu-Hyp-D-Phe-Arg-Trp-Lys]-NH₂

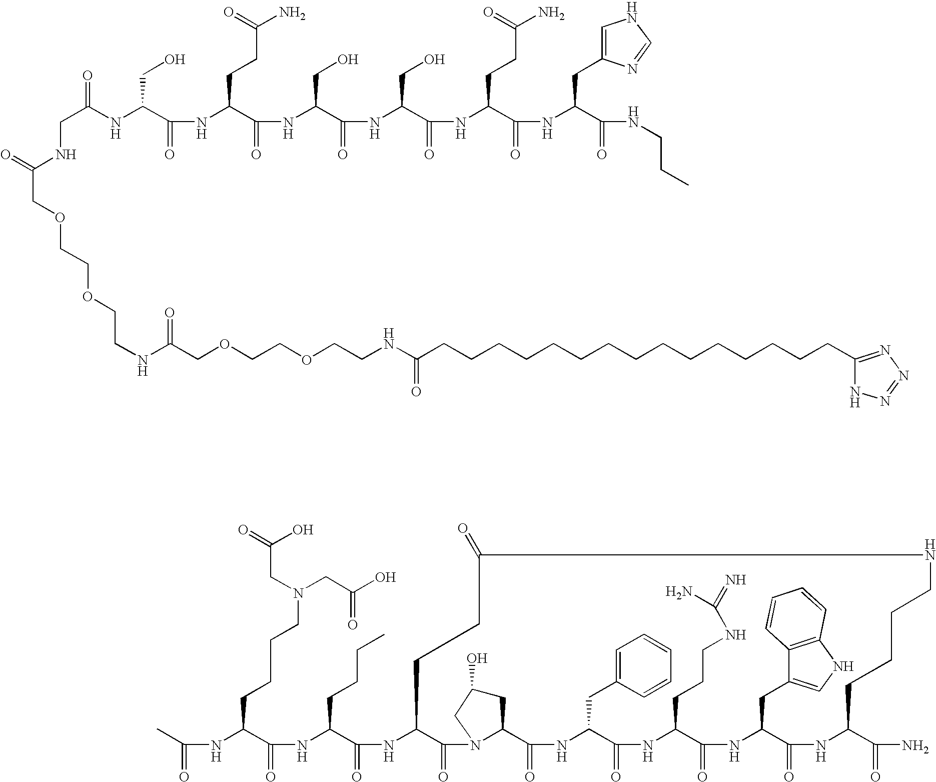

The compound was prepared similarly to the procedure described for Example 3.

LCMS (system 2): Rt=5.08 min; ((m+2)/2)=1299.3

### Example 21

### {2-[2-(15-Carboxypentadecanoylamino)ethoxy]ethoxy}acetyl-Gly-Ser-Gln-Ser-Lys(BCMA)-Nle-c[Glu-Hyp-D-Phe-Arg-Trp-Lys]-NH₂

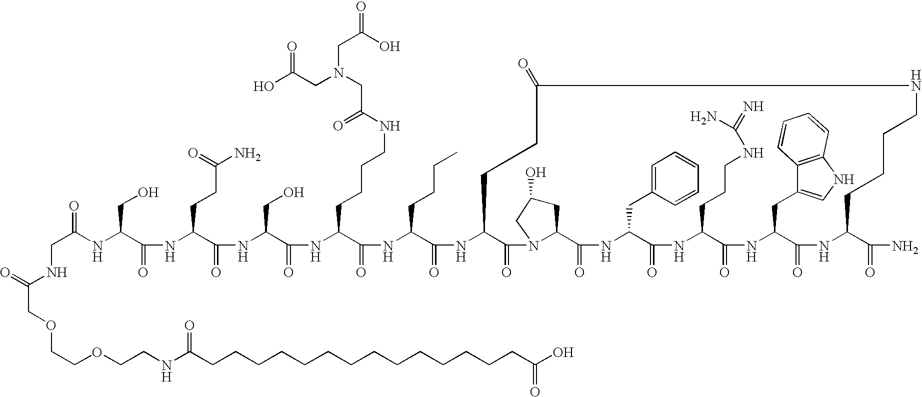

The compound was prepared similarly to the procedure described for Example 1.

LCMS (system 1): Rt=2.24 min; ((m+2)/2)=1024.0

### Example 22

### {2-[2-(2-{2-[2-(19-Carboxynonadecanoylamino)ethoxy]ethoxy}acetylamino)ethoxy]ethoxy}acetyl-Gly-D-Ser-Gln-Ser-Ser-Gln-His-Lys(biscarboxymethyl)β-Ala-Nle-c[Glu-Hyp-D-Phe-Arg-Trp-Lys]-NH₂

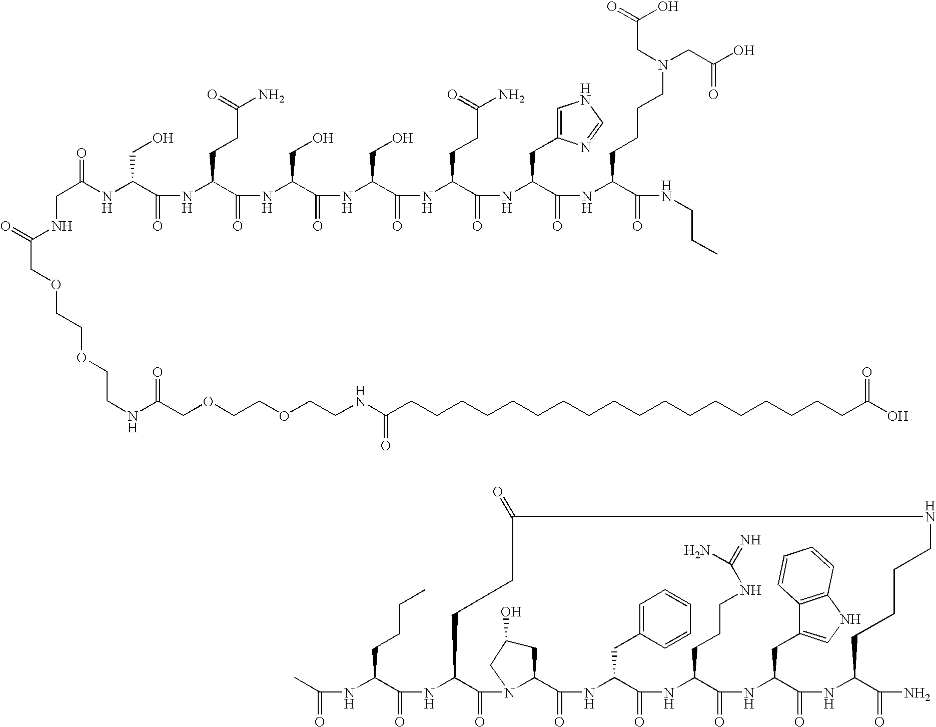

The compound was prepared similarly to the procedure described for Example 3 by using the building block icosanedioic acid mono-tert-butyl ester (available by the synthetic procedure described in: U. Widmer, Synthesis 1983, 135).

LCMS (system 2): Rt=5.35 min; ((m+2)/2)=1308.2

### Example 23

### (2-{2-[16-(Tetrazol-5-yl)hexadecanoylamino]ethoxy}ethoxy)acetyl-Gly-Ser-Gln-Tyr-Dap(biscarboxymethyl)-Nle-c[Glu-Hyp-D-Phe-Arg-Trp-Lys]-NH₂

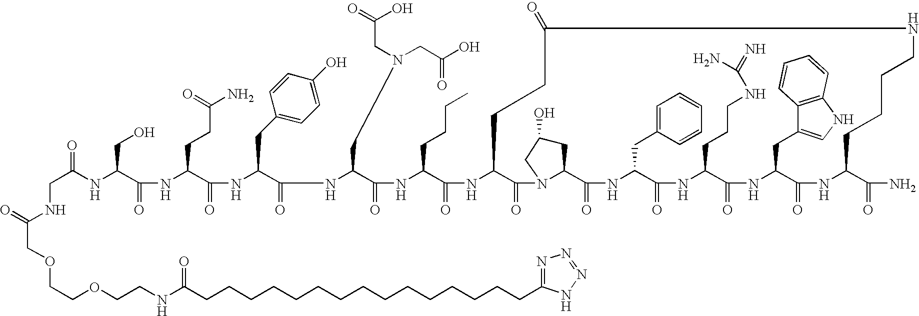

The compound was prepared similarly to the procedure described for Example 3.

LCMS (system 1): Rt=2.31 min; ((m+2)/2)=1031.5

### Example 24

### Solubility Data of Compounds in Water

From a stock solution in H₂O containing 1 mg/ml of compound, 8-11 aliquots are withdrawn and pH-adjusted individually with HAc/NaOH, to cover the whole pH range. After incubation at room temperature for 3 days, the samples are centrifuged at 20.000 g for 20 min., the pH is measured and the solubility determined by quantification of content in the supernatant by UV-detection (e₍₂₈₀ₙₘ₎=5500M⁻¹cm⁻¹).

(2{2-[16-(Tetrazol-5-yl)hexadecanoylamino]ethoxy}ethoxy)acetyl-Gly-
Ser-Gln-His-Dap(BCMA)-Nle-c[Glu-Hyp-D-Phe-Arg-Trp-Lys]—NH₂

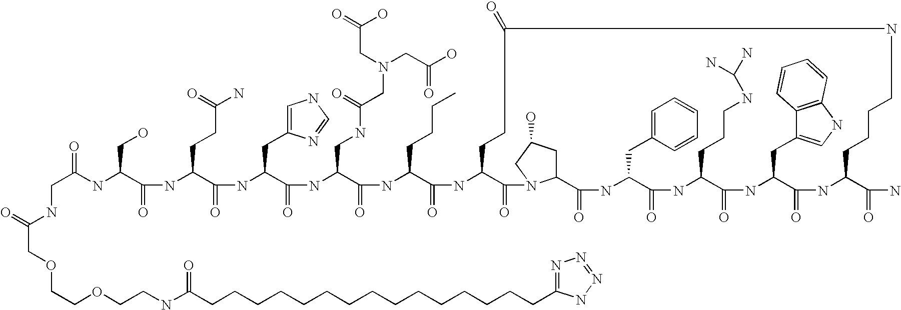

| pH | Conc (μM) |
|---|---|
| 7.71 | 796 |
| 7.39 | 735 |
| 7.15 | 493 |
| 6.74 | 70 |
| 5.82 | 56 |
| 5.27 | 60 |
| 4.64 | 78 |
| 4.29 | 380 |
| 4.02 | 749 |
| 3.13 | 725 |

(2-{2-[16-(Tetrazol-5-yl)hexadecanoylamino]ethoxy}ethoxy)acetyl-Gly-
Ser-Gln-His-Dap(biscarboxymethyl)-Nle-c[Glu-Hyp-D-Phe-Arg-Trp-Lys]—NH₂

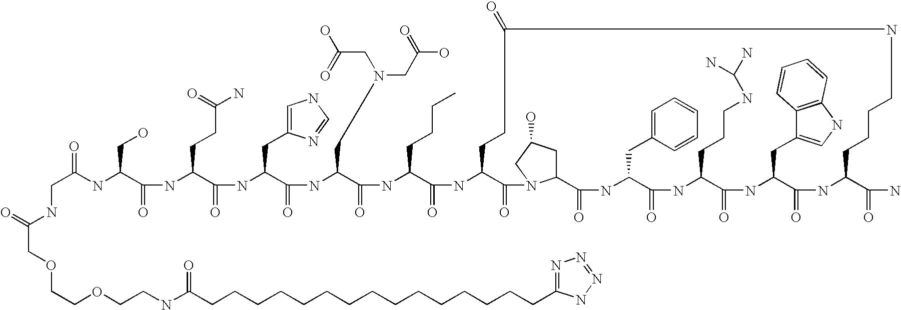

| pH | Conc (μM) |
|---|---|
| 7.74 | 584 |
| 7.28 | 544 |
| 7.11 | 349 |
| 6.71 | 46 |
| 5.60 | 16 |
| 5.59 | 17 |
| 4.85 | 425 |
| 4.66 | 640 |
| 4.24 | 703 |
| 3.91 | 747 |

(2-{2-[16-(Tetrazol-5-yl)hexadecanoylamino]ethoxy}ethoxy)acetyl-Gly-
Ser-Gln-His-Lys(BCMA)-Nle-c[Glu-Hyp-D-Phe-Arg-Trp-Lys]—NH₂

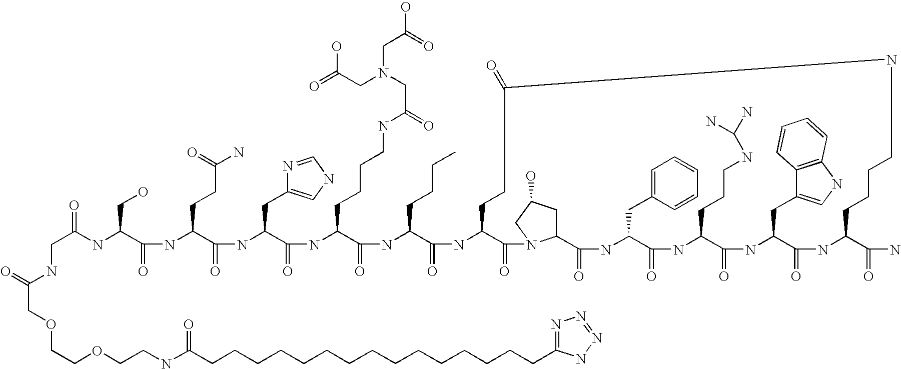

| pH | Conc (μM) |
|---|---|
| 7.81 | 1060 |
| 7.66 | 970 |
| 7.23 | 680 |
| 7.1 | 340 |
| 6.63 | 30 |
| 5.63 | 0 |
| 4.7 | 0 |
| 4.31 | 240 |
| 4.06 | 880 |
| 3.86 | 970 |
| 2.88 | 1040 |

[2-(2-{4-[16-(Tetrazol-5-
yl)hexadecanoylsulfamoyl]butanoylamino)ethoxy)ethoxy]acetyl-Gly-Ser-Gln-His-
Dap(BCMA)-Nle-c[Glu-Hyp-D-Phe-Arg-Trp-Lys]—NH₂

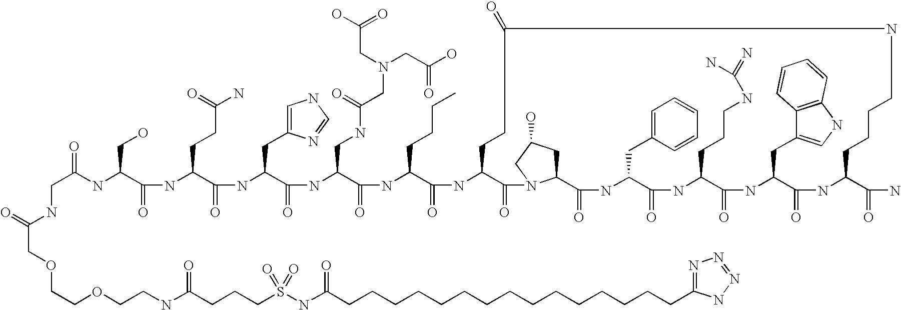

| pH | Conc (μM) |
|---|---|
| 7.8 | 1040 |
| 7.48 | 830 |
| 7.18 | 550 |
| 6.94 | 280 |
| 6.36 | 45 |
| 4.31 | 57 |
| 3.9 | 770 |
| 3.72 | 910 |
| 2.86 | 1030 |

(2-{2-[16-(Tetrazol-5-yl)hexadecanoylamino]ethoxy}ethoxy)acetyl-Gly-
Ser-Gln-Tyr-Dap(biscarboxymethyl)-Nle-c[Glu-Hyp-D-Phe-Arg-Trp-Lys]—NH₂

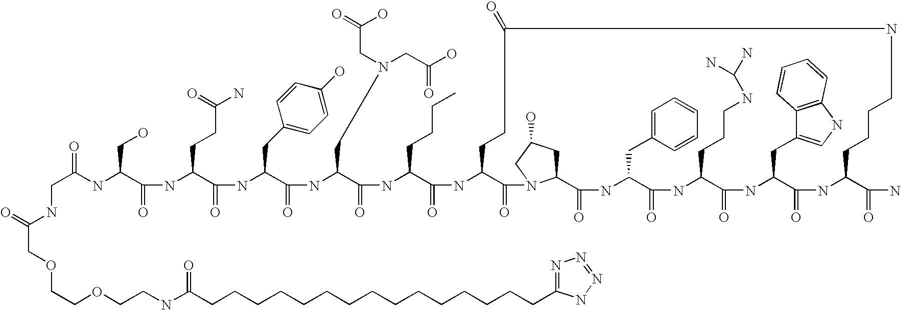

| pH | Conc (μM) |
|---|---|
| 8.48 | 450 |
| 8.18 | 370 |
| 7.9 | 450 |
| 7.42 | 290 |
| 6.92 | 160 |
| 5.94 | 16 |
| 5.04 | 8 |
| 4.69 | 11 |
| 4.18 | 9 |
| 3.83 | 8 |

(2-{2-[16-(Tetrazol-5-yl)hexadecanoylamino]ethoxy}ethoxy)acetyl-Gly-
Ser-Gln-Ser-Dap(BCMA)-Nle-c[Glu-Hyp-D-Phe-Arg-Trp-Lys]—NH₂

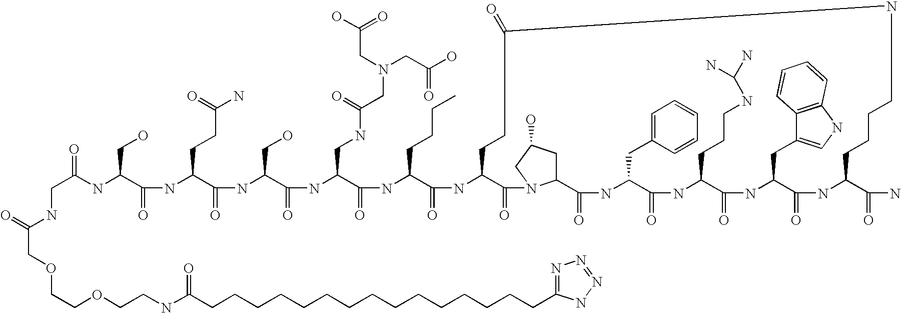

| pH | Conc (μM) |
|---|---|
| 7.42 | 290 |
| 7.19 | 160 |
| 6.12 | 80 |
| 5.67 | 14 |
| 5.15 | 4.5 |
| 4.87 | 10 |
| 4.57 | 5 |
| 3.69 | 8 |
| 3.17 | 5 |

(2-{2-[16-(Tetrazol-5-yl)hexadecanoylamino]ethoxy}ethoxy)acetyl-Gly-
Ser-Gln-His-Dab(BCMA)-Nle-c[Glu-Hyp-D-Phe-Arg-Trp-Lys]—NH₂

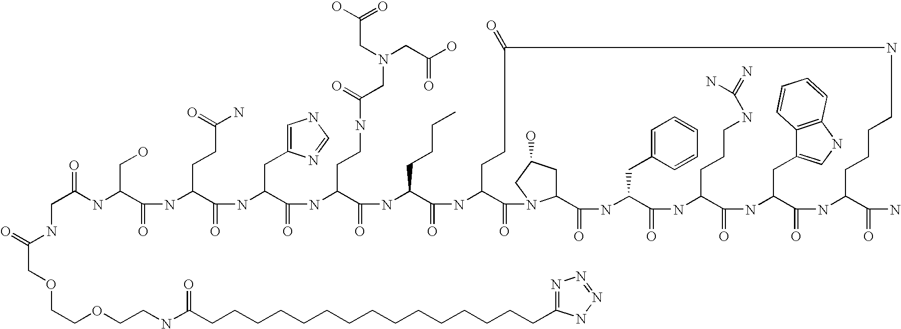

| pH | Conc (μM) |
|---|---|
| 7.87 | 702 |
| 7.41 | 562 |
| 7.26 | 475 |
| 6.99 | 151 |
| 6.75 | 37 |
| 5.63 | 14 |
| 4.94 | 32 |
| 4.42 | 424 |
| 4.23 | 689 |
| 2.92 | 708 |

{2-[2-(15-Carboxypentadacanoylamino)ethoxy]ethoxy}acetyl-Gly-Ser-
Gln-His-Dap(BCMA)-Nle-c[Glu-Hyp-D-Phe-Arg-Trp-Lys]—NH₂

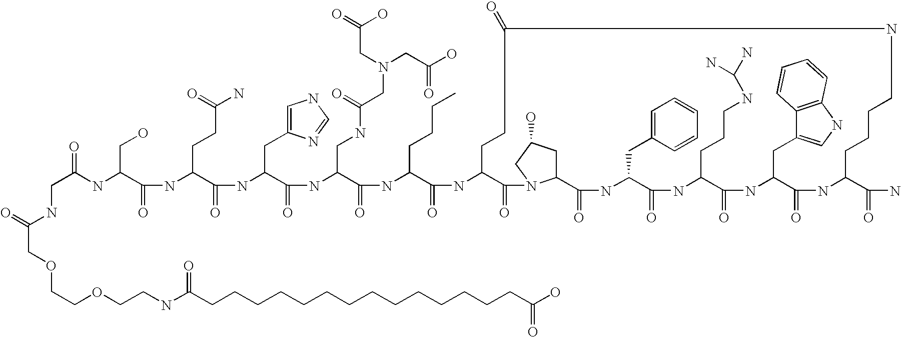

| pH | Conc (μM) |
|---|---|
| 7.59 | 526 |
| 7.25 | 489 |
| 7.23 | 451 |
| 6.99 | 346 |
| 6.58 | 198 |
| 5.56 | 64 |
| 4.89 | 107 |
| 4.53 | 436 |
| 4.24 | 569 |
| 3.04 | 567 |

(2-{2-[16-(Tetrazol-5-yl)hexadecanoylamino]ethoxy}ethoxy)acetyl-Gly-
Ser-Gln-His-β-Dap(BCMA)-Nle-c[Glu-Hyp-D-Phe-Arg-Trp-Lys]—NH₂

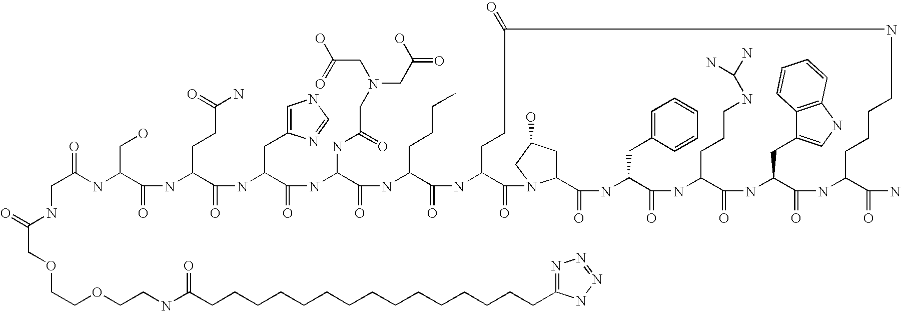

| pH | Conc (μM) |
|---|---|
| 7.81 | 77 |
| 7.53 | 320 |
| 7.36 | 251 |
| 7.2 | 122 |
| 6.55 | 14 |
| 5.22 | 13 |
| 4.64 | 49 |
| 4.3 | 216 |
| 3.84 | 421 |
| 3.02 | 320 |

(2-{2-[16-(Tetrazol-5-yl)hexadecanoylamino]ethoxy}ethoxy)acetyl-Gly-
Ser-Gln-His-Orn(BCMA)-Nle-c[Glu-Hyp-D-Phe-Arg-Trp-Lys]—NH₂

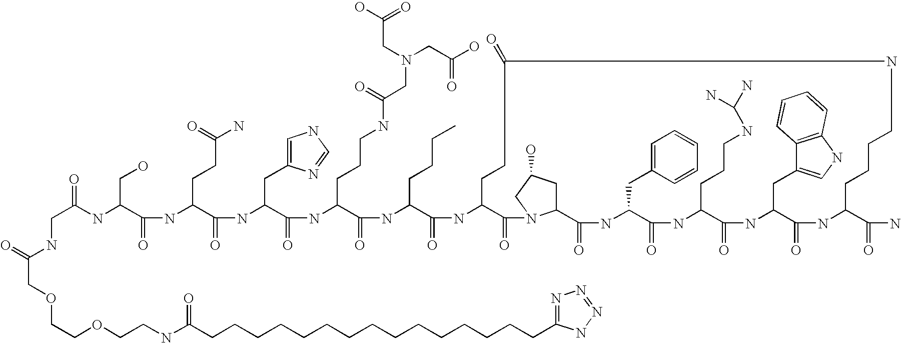

| pH | Conc (μM) |
|---|---|
| 7.95 | 546 |
| 7.75 | 280 |
| 7.47 | 116 |
| 7.45 | 271 |
| 6.7 | 26 |
| 5.9 | 10 |
| 4.87 | 34 |
| 4.24 | 389 |
| 4.1 | 536 |
| 2.96 | 716 |

(2-{2-[16-(Tetrazol-5-yl)hexadecanoylamino]ethoxy}ethoxy)acetyl-Gly-
Ser-Gln-His-Lys(biscarboxymethyl)-Nle-c[Glu-Hyp-D-Phe-Arg-Trp-Lys]—NH₂

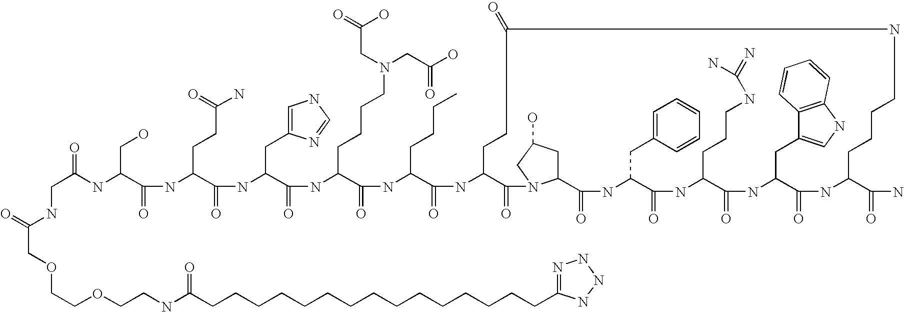

| pH | Conc (μM) |
|---|---|
| 7.78 | 391 |
| 7.48 | 262 |
| 6.98 | 35 |
| 6.48 | 18 |
| 5.64 | 19 |
| 4.75 | 42 |
| 3.95 | 276 |
| 3.59 | 398 |
| 3.28 | 476 |
| 2.94 | 536 |

(2-{2-[2-(2-{2-[2-(2-{2-[2-(2-{2-[16-(Tetrazol-5-yl)hexadecanoylamino]ethoxy}eth-
oxy)acetylamino]ethoxy}ethoxy)acetylamino]ethoxy}ethoxy)acetylamino]ethoxy}ethoxy)acetyl-
Gly-Ser-Gln-Ser-Dap(BCMA)-Nle-c[Glu-Hyp-D-Phe-Arg-Trp-Lys]—NH₂

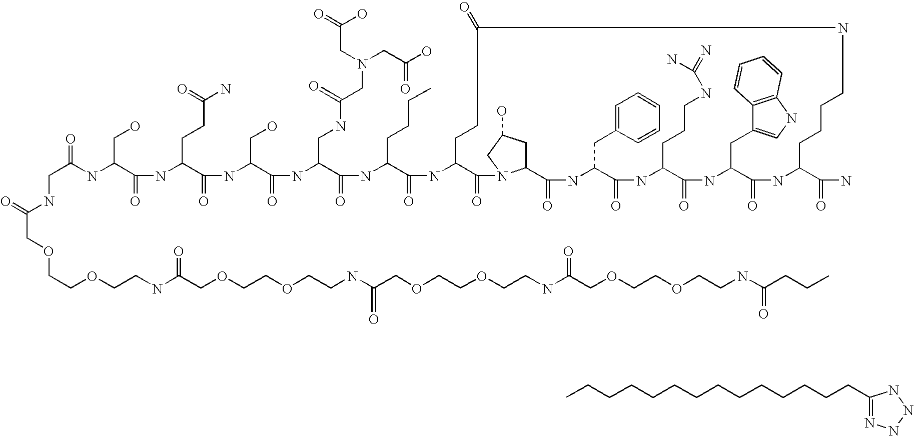

| pH | Conc (μM) |
|---|---|
| 7.43 | 29 |
| 7.29 | 20 |
| 7.00 | 17 |
| 6.50 | 19 |
| 5.84 | 41 |
| 5.37 | 10 |
| 5.07 | 18 |
| 4.66 | 23 |
| 4.62 | 15 |
| 3.18 | 19 |

(2-{2-[16-(Tetrazol-5-yl)hexadecanoylamino]ethoxy}ethoxy)acetyl-Gly-
Ser-Gln-Ser-Lys(BCMA)-Nle-c[Glu-Hyp-D-Phe-Arg-Trp-Lys]—NH₂

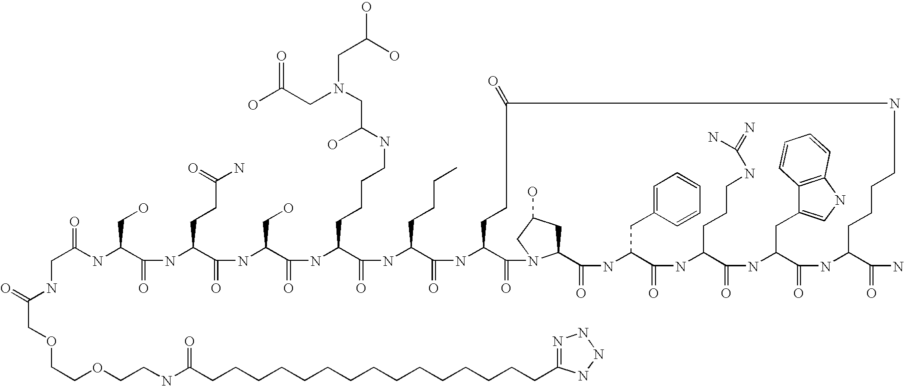

| pH | Conc (μM) |
|---|---|
| 7.40 | 400 |
| 7.11 | 403 |
| 6.51 | 395 |
| 6.40 | 392 |
| 6.04 | 369 |
| 5.76 | 208 |
| 5.61 | 97 |
| 5.24 | 30 |
| 4.66 | 13 |
| 4.11 | 13 |

(2-{2-[2-(2-{2-[16-(Tetrazol-5-
yl)hexadecanoylamino]ethoxy}ethoxy)acetylamino]ethoxy}ethoxy)acetyl-Gly-D-
Ser-Gln-Ser-Ser-Gln-His-β-Ala-Lys(biscarboxymethyl)-Nle-c[Glu-Hyp-D-Phe-
Arg-Trp-Lys]—NH₂

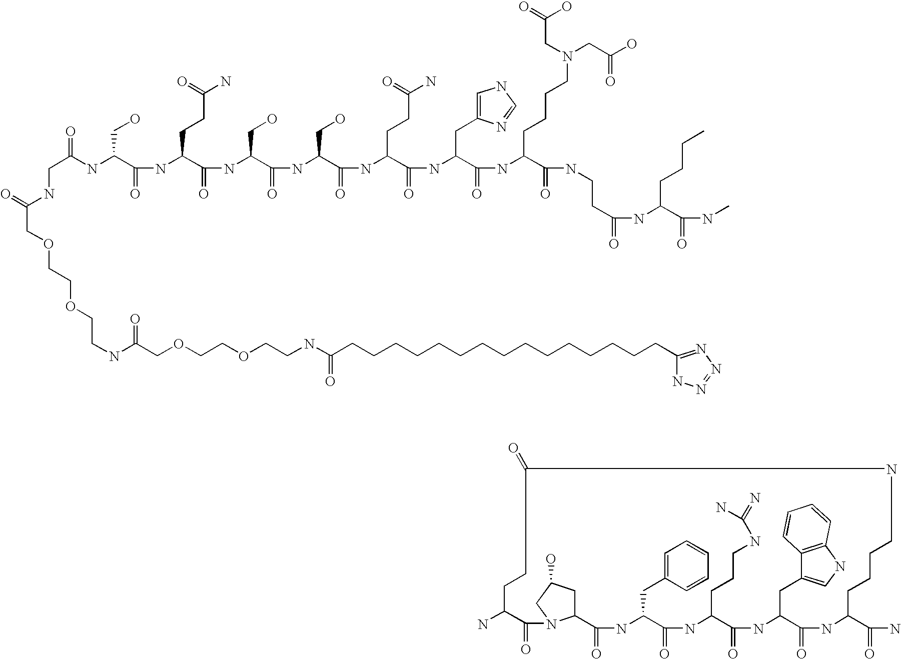

| pH | Conc (μM) |
|---|---|
| 7.72 | 256 |
| 7.15 | 210 |
| 6.99 | 119 |
| 6.71 | 58 |
| 6.71 | 87 |
| 5.56 | 43 |
| 5.07 | 136 |
| 4.82 | 268 |
| 4.69 | 395 |
| 4.11 | 374 |
| 3.10 | 409 |

(2-{2-[16-(Tetrazol-5-yl)hexadecanoylamino]ethoxy}ethoxy)acetyl-Gly-
Ser-Gln-Ser-Lys(biscarboxymethyl)-Nle-c[Glu-Hyp-D-Phe-Arg-Trp-Lys]—NH₂

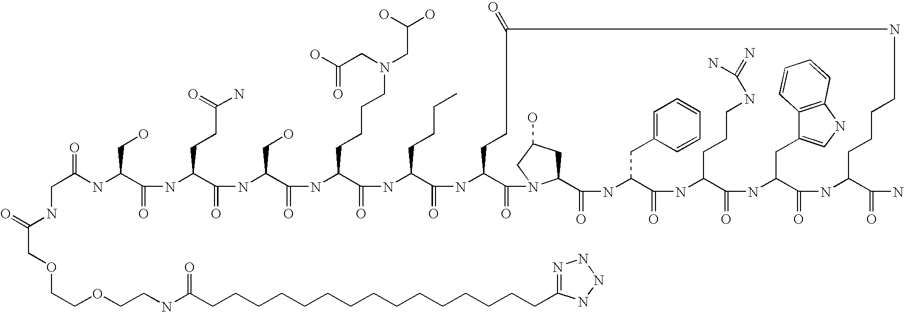

| pH | Conc (μM) |
|---|---|
| 7.03 | 245 |
| 7.04 | 251 |
| 6.68 | 244 |
| 6.29 | 239 |
| 5.97 | 129 |
| 5.23 | 30 |
| 5.10 | 25 |
| 4.55 | 21 |
| 4.22 | 20 |

{2-[2-(15-Carboxypentadecanoylamino)ethoxy]ethoxy}acetyl-Gly-Ser-
Gln-Ser-Lys(BCMA)-Nle-c[Glu-Hyp-D-Phe-Arg-Trp-Lys]—NH₂

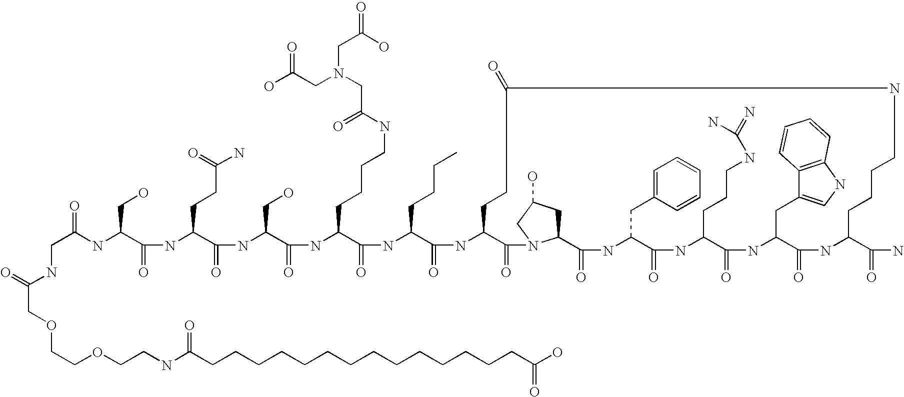

| pH | Conc (μM) |
|---|---|
| 7.61 | 107 |
| 7.60 | 116 |
| 6.87 | 117 |
| 6.29 | 106 |
| 6.26 | 108 |
| 5.67 | 103 |
| 5.21 | 58 |
| 4.74 | 40 |
| 4.34 | 32 |
| 4.04 | 26 |

(2-{2-[2-(2-{2-[2-(2-{2-[2-(2-{2-[16-(Tetrazol-5-
yl)hexadecanoylamino]ethoxy}ethoxy)acetylamino]ethoxy}
ethoxy)acetylamino]ethoxy}ethoxy)acetylamino]ethoxy}ethoxy)acetyl-Gly-Ser-
Gln-His-Dap(BCMA)-Nle-c[Glu-Hyp-D-Phe-Arg-Trp-Lys]—NH₂

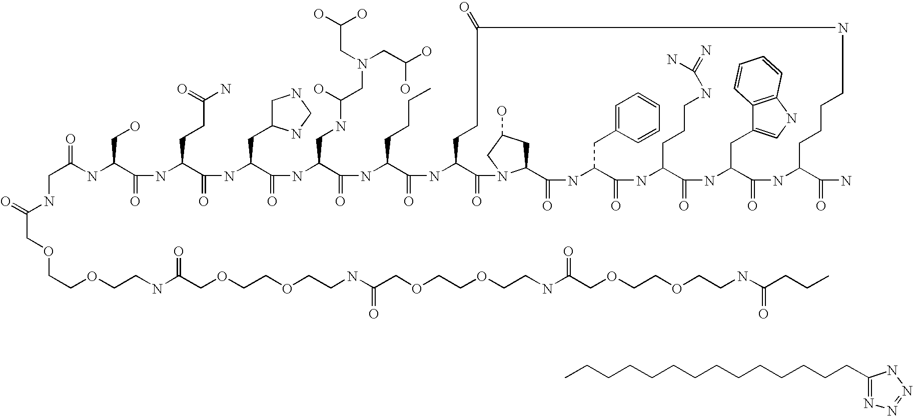

| pH | Conc (μM) |
|---|---|
| 7.66 | 596 |
| 7.30 | 454 |
| 6.66 | 93 |
| 6.37 | 51 |
| 5.75 | 41 |
| 4.98 | 347 |
| 4.89 | 450 |
| 4.67 | 492 |
| 4.32 | 515 |
| 3.18 | 531 |

{2-[2-(2-{2-[2-(19-
Carboxynonadecanoylamino)ethoxy]ethoxy}acetylamino)ethoxy]ethoxy}acetyl-
Gly-D-Ser-Gln-Ser-Ser-Gln-His-Lys(biscarboxymethyl)-β-Ala-Nle-c[Glu-Hyp-D-
Phe-Arg-Trp-Lys]—NH₂

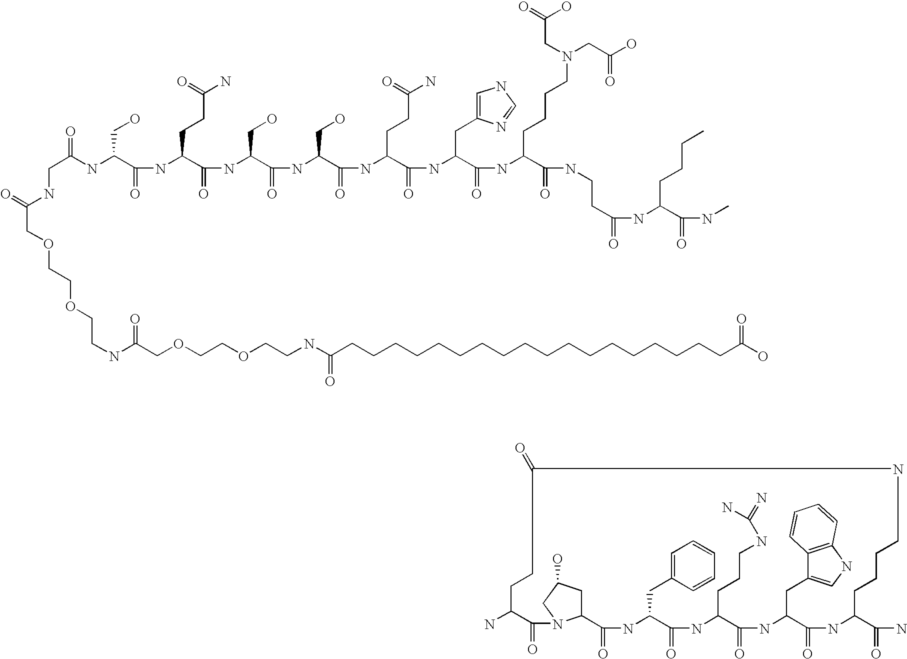

| pH | Conc (μM) |
|---|---|
| 7.65 | 704 |
| 7.40 | 57 |
| 7.28 | 40 |
| 6.53 | 12 |
| 6.46 | 12 |
| 5.60 | 62 |
| 5.18 | 224 |
| 4.68 | 363 |
| 4.09 | 609 |
| 3.11 | 405 |

(2-{2-[2-(2-{2-[16-(Tetrazol-5-
yl)hexadecanoylamino]ethoxy}ethoxy)acetylamino]ethoxy}ethoxy)acetyl-Gly-D-
Ser-Gln-Ser-Ser-Gln-His-β-Ala-Lys(biscarboxymethyl)-Nle-c[Glu-Hyp-D-Phe-
Arg-Trp-Lys]—NH₂

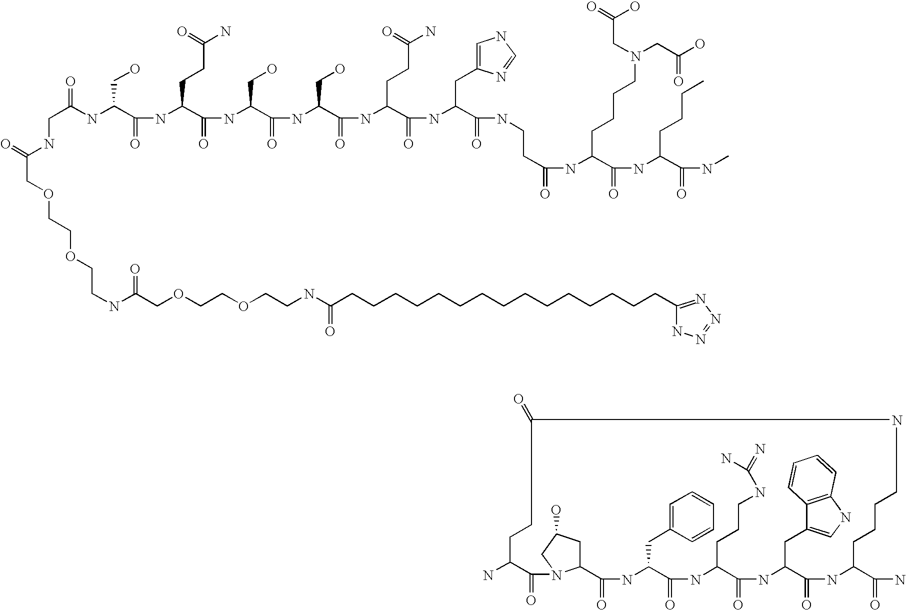

| pH | Conc (μM) |
|---|---|
| 7.55 | 452 |
| 7.23 | 473 |
| 6.99 | 345 |
| 6.91 | 223 |
| 6.23 | 53 |
| 5.51 | 56 |
| 5.40 | 67 |
| 4.97 | 406 |
| 4.37 | 512 |
| 2.94 | 499 |

(2-{2-[16-(Tetrazol-5-yl)hexadecanoylamino]ethoxy}ethoxy)acetyl-Glu-
Ser-Gln-His-Dap(BCMA)-Nle-c[Glu-Hyp-D-Phe-Arg-Trp-Lys]—NH₂

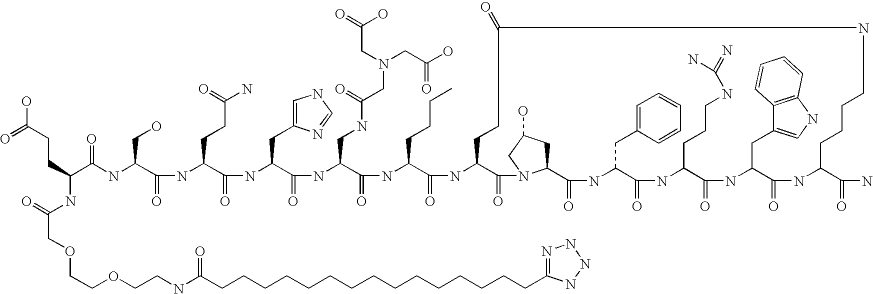

| pH | Conc (μM) |
|---|---|
| 7.62 | 351 |
| 6.89 | 303 |
| 6.34 | 226 |
| 6.04 | 127 |
| 5.55 | 39 |
| 4.94 | 23 |
| 4.56 | 20 |
| 4.31 | 20 |
| 3.76 | 25 |
| 3.32 | 34 |

(2-{2-[2-(2-{2-[16-(Tetrazol-5-
yl)hexadecanoylamino]ethoxy}ethoxy)acetylamino]ethoxy}ethoxy)acetyl-Gly-D-
Ser-Gln-Ser-Ser-Gln-His-Lys(biscarboxymethyl)-Nle-c[Glu-Hyp-D-Phe-Arg-
Trp-Lys]—NH₂

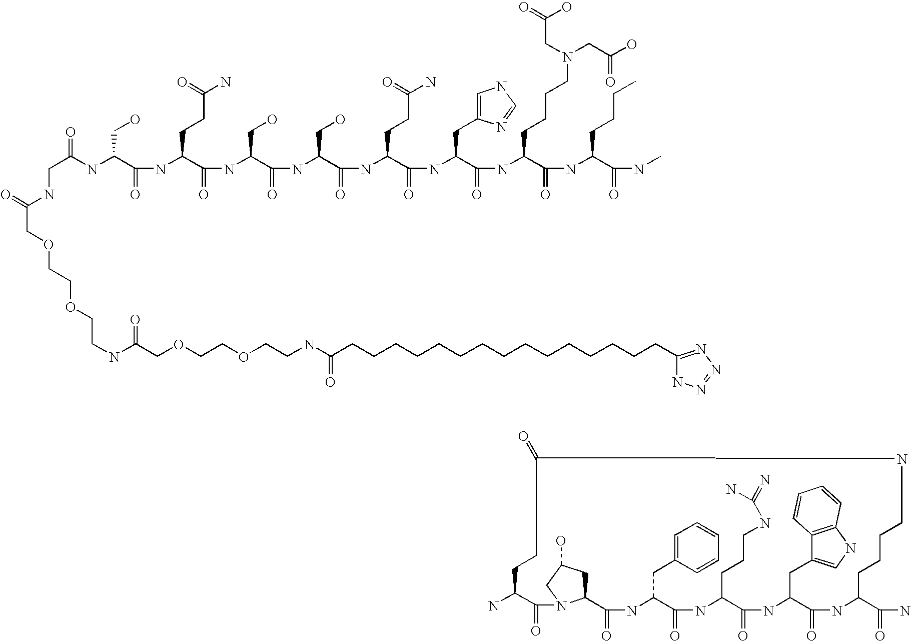

| pH | Conc (μM) |
|---|---|
| 7.29 | 316 |
| 7.24 | 256 |
| 7.15 | 190 |
| 6.95 | 87 |
| 6.10 | 39 |
| 5.18 | 75 |
| 4.84 | 324 |
| 4.82 | 429 |
| 3.88 | 570 |
| 2.95 | 435 |

(2-{2-[2-(2-{2-[(S)-4-Carboxy-4-(17-
carboxyheptadecanoylamino)butanoylamino]ethoxy}ethoxy)acetylamino]ethoxy}ethoxy)acetyl-
Gly-Ser-Gln-His-Dap(BCMA)-Nle-c[Glu-Hyp-D-Phe-Arg-Trp-Lys]—NH₂

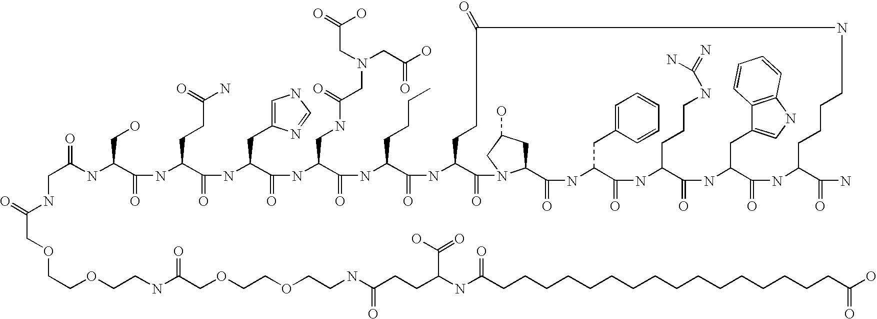

| pH | Conc (μM) |
|---|---|
| 7.51 | 498 |
| 7.07 | 485 |
| 6.95 | 382 |
| 6.73 | 342 |
| 6.56 | 249 |
| 5.96 | 78 |
| 5.20 | 25 |
| 3.70 | 391 |

### Pharmacological Methods

Assay (I)—Experimental protocol for efficacy testing on appetite with MC4 analogues, using an ad libitum fed rat model.

TAC:SPRD @mol rats or Wistar rats from M&B Breeding and Research Centre A/S, Denmark are used for the experiments. The rats have a body weight 200-250 g at the start of experiment. The rats arrive at least 10-14 days before start of experiment with a body weight of 180-200 g. Each dose of compound is tested in a group of 8 rats. A vehicle group of 8 rats is included in each set of testing.

When the animals arrive they are housed individually in a reversed light/dark phase (lights off 7:30 am, lights on 7:30 pm), meaning that lights are off during daytime and on during nighttime. Since rats normally initiate food intake when light is removed, and eat the major part of their daily food intake during the night, this set up results in an alteration of the initiation time for food intake to 7:30 am, when lights are switched off. During the acclimatization period of 10-14 days, the rats have free access to food and water. During this period the animals are handled at least 3 times. The experiment is conducted in the rats' home cages. Immediately before dosing the rats are randomised to the various treatment groups (n 8) by body weight. They are dosed according to body weight at between 7:00 am and 7:45 am, with a 1-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually every hour for 7 hours, and then after 24 h and sometimes 48 h. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

TABLE 1
In vivo efficacy testing on appetite dosing 3 mg/kg of MC4 agonists

| | | | Assay | |
|---|---|---|---|---|
| | | | (I) | |
| | | | Food | |
| | | | con- | |
| | | | sump- | |
| | | | tion | |
| | | | (% of | |
| | | | vehicle) | |
| Example | | | 24 | 48 |
| nr. | Compound | Molecule | h | h |
| Example 1 | (2-{2-[16- (Tetrazol-5- yl) hexadecanoylamino] ethoxy}ethoxy) acetyl-Gly-Ser-Gln-His- Lys(BCMA)-Nle- c[Glu-Hyp-D-Phe- Arg-Trp-Lys]—NH₂ | 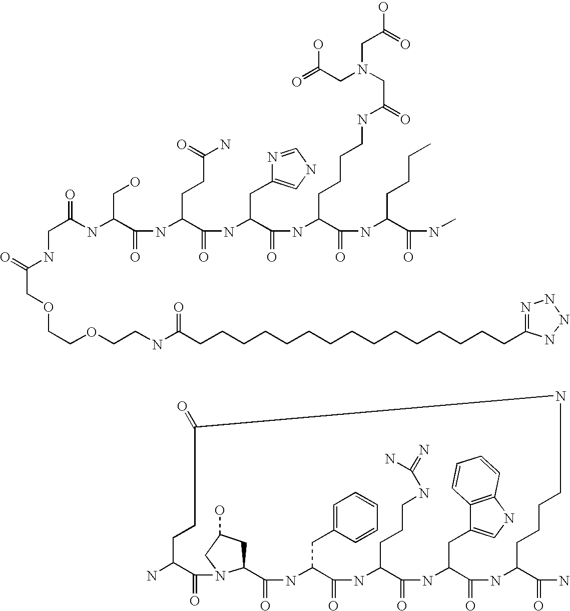 | 65 | 72 |
| Example 4 | (2-{2-[16- (Tetrazol-5- yl) hexadecanoylamino] ethoxy}ethoxy) acetyl-Gly-Ser-Gln-Ser- Dap(BCMA)-Nle- c[Glu-Hyp-D-Phe- Arg-Trp-Lys]—NH₂ | 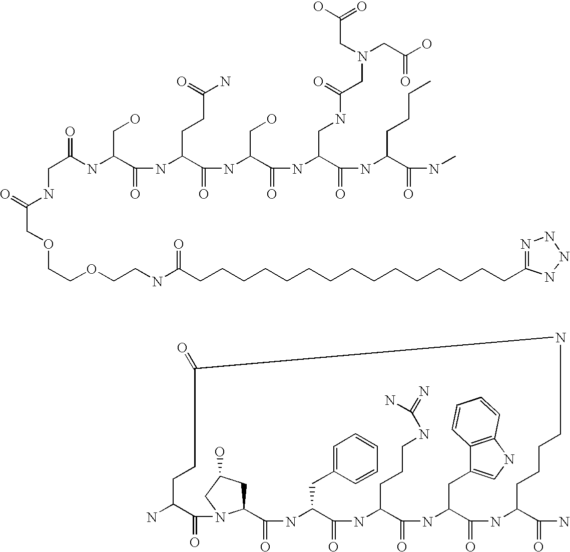 | 52 | 69 |
| Example 6 | (2-{2-[16- (Tetrazol-5- yl) hexadecanoylamino] ethoxy}ethoxy) acetyl-Gly-Ser-Gln-His- Dap (biscarboxymethyl)- Nle-c[Glu-Hyp-D- Phe-Arg-Trp-Lys]— NH₂ | 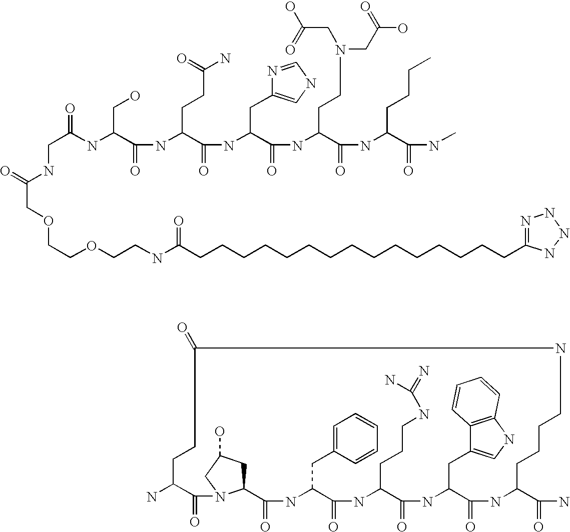 | 58 | 69 |
| Example 9 | (2-{2-[16- (Tetrazol-5- yl) hexadecanoylamino] ethoxy}ethoxy) acetyl-Gly-Ser-Gln- His-Dap(BCMA)-Nle- c[Glu-Hyp-D-Phe- Arg-Trp-Lys]—NH₂ | 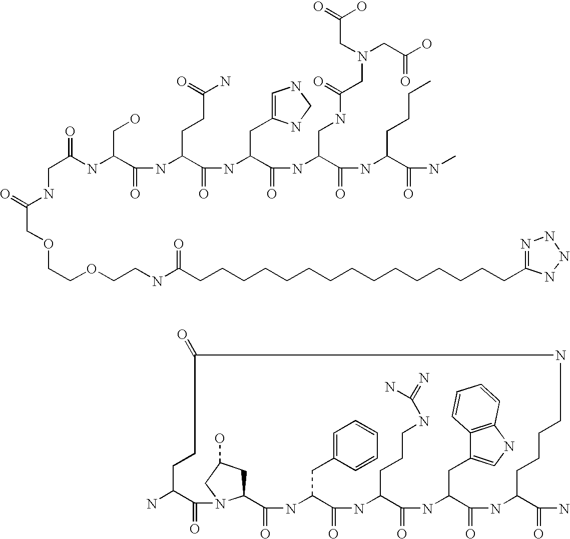 | 47 | 57 |

TABLE 2
In vivo efficacy testing on appetite of dosing 1 mg/kg of MC4 agonists

Assay
(I)
Food
con-
sump-
tion
(% of
vehicle)

| Exam- | | | 24 | 48 |
|---|---|---|---|---|
| ple nr. | Compound | Molecule | h | h |
| Exam- ple 2 | (2-{2-[16- (Tetrazol-5- yl) hexadecanoylamino] ethoxy}ethoxy) acetyl-Gly- Ser-Gln-His-β- Dap(BCMA)- Nle-c[Glu- Hyp-D-Phe-Arg-Trp- Lys]—NH₂ | 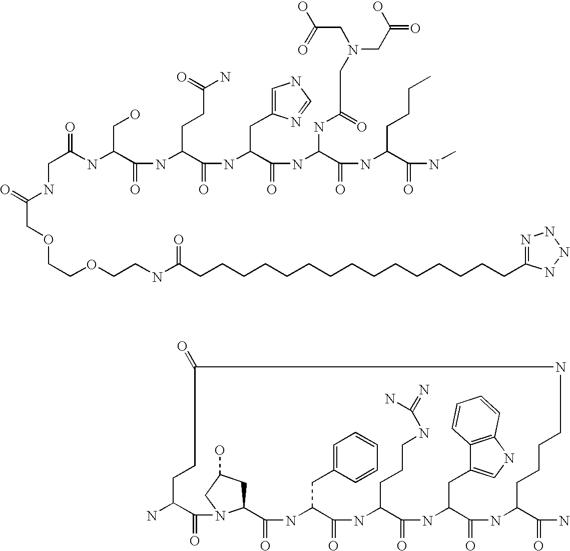 | 60 | 71 |
| Exam- ple 5 | (2-{2-(16- (Tetrazol-5- yl) hexadecanoylamino] ethoxy}ethoxy) acetyl-Gly- Ser-Gln-Ser-Lys (biscarboxymethyl)- Nle-c[Glu-Hyp-D-Phe- Arg-Trp-Lys]—NH₂ | 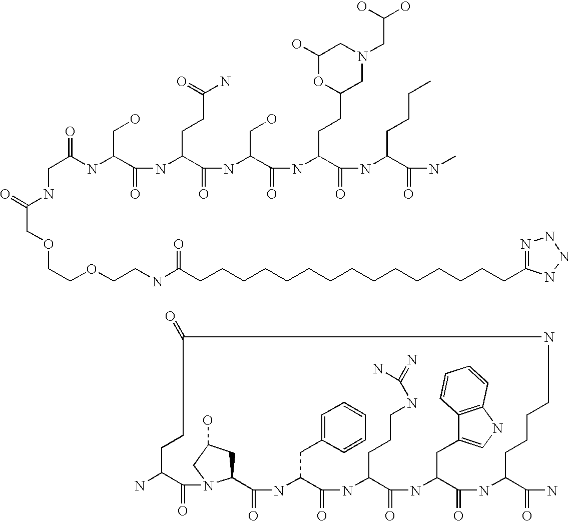 | 81 | 91 |
| Exam- ple 8 | (2-{2-[16- (Tetrazol-5- yl) hexadecanoylamino] ethoxy}ethoxy) acetyl-Gly- Ser-Gln-His- Orn(BCMA)-Nle-c[Glu- Hyp-D-Phe-Arg-Trp- Lys]—NH₂ | 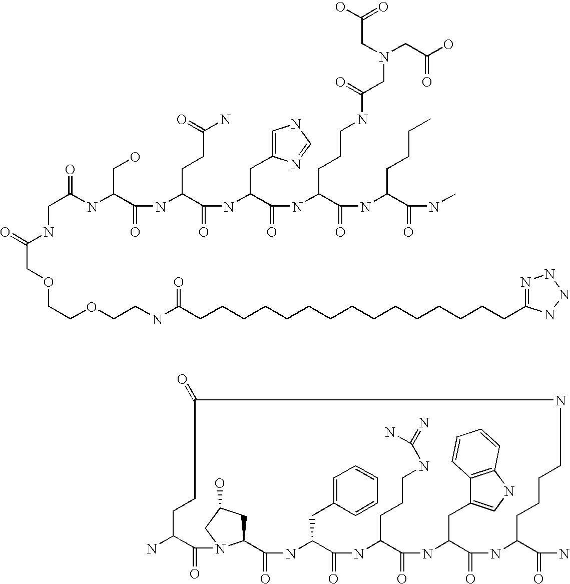 | 62 | 75 |
| Exam- ple 12 | (2-{2-[2-(2-{2- [16-(Tetrazol-5- yl) hexadecanoylamino] ethoxy}ethoxy) acetylamino] ethoxy}ethoxy)acetyl- Gly-D-Ser-Gln- Ser-Ser-Gln- His-β- Ala-Lys (biscarboxymethyl)- Nle-c[Glu-Hyp-D-Phe- Arg-Trp-Lys]—NH₂ | 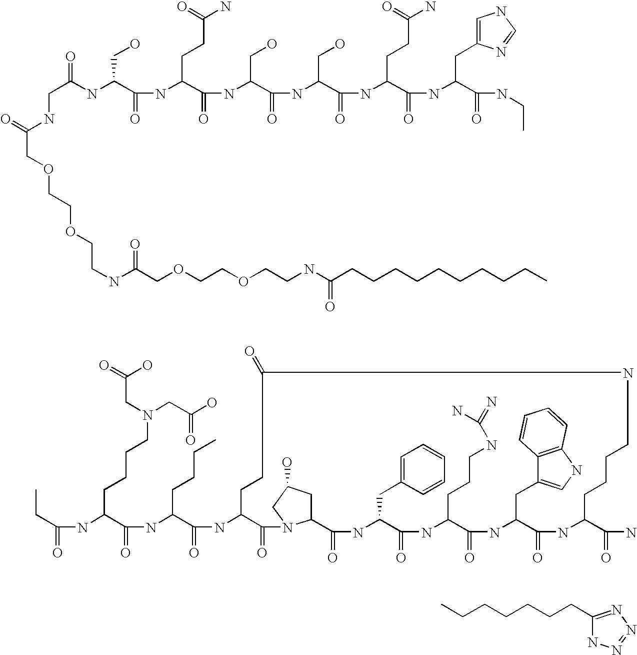 | 62 | 77 |
| Exam- ple 13 | {2-[2-(15- Carboxypentade- canoylamino)ethoxy] ethoxy}acetyl- Gly-Ser-Gln- His-Dap(BCMA)-Nle- c[Glu-Hyp- D-Phe-Arg- Trp-Lys]—NH₂ | 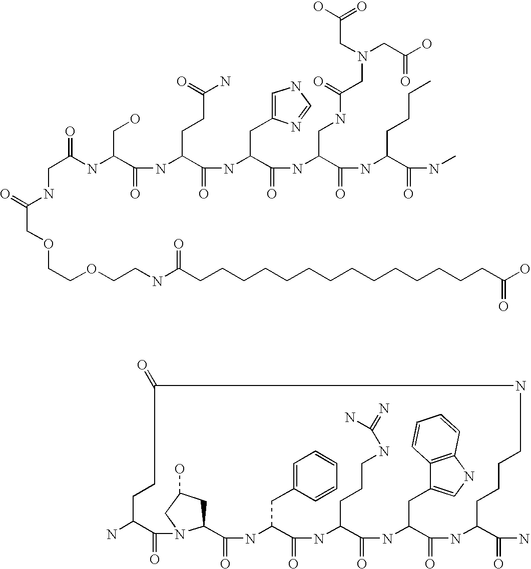 | 59 | 80 |
| Exam- ple 15 | (2-{2-[16- (Tetrazol-5- yl) hexadecanoylamino] ethoxy}ethoxy)acetyl- Gly-Ser-Gln-Ser- Lys(BCMA)- Nle-c[Glu- Hyp-D-Phe-Arg-Trp- Lys]—NH₂ | 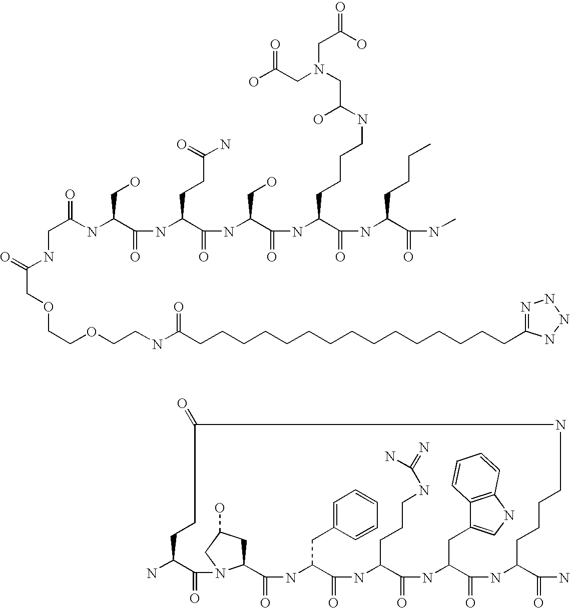 | 74 | 85 |
| Exam- ple 20 | (2-{2-[2-(2-{2- [16-(Tetrazol-5- yl) hexadecanoylamino] ethoxy}ethoxy) acetylamino] ethoxy}ethoxy)acetyl- Gly-D-Ser- Gln-Ser-Ser- Gln-His-β-Ala- Lys (biscarboxymethyl)- Nle-c[Glu-Hyp-D-Phe- Arg-Trp-Lys]—NH₂ | 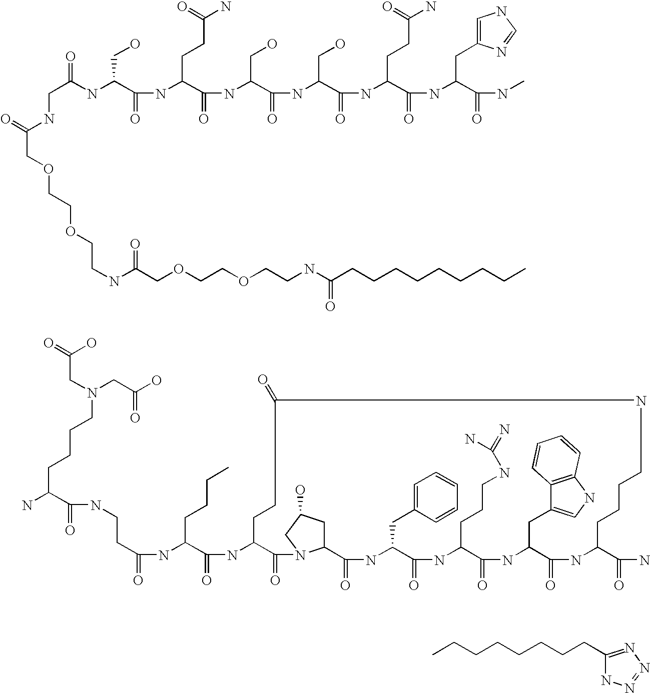 | 61 | 72 |
| Exam- ple 22 | {2-[2-(2-{2-[2- (19-Carboxynon- adecanoylamino) ethoxy]ethoxy} acetylamino)ethoxy] ethoxy}acetyl-Gly-D- Ser-Gln-Ser- Ser-Gln-His-Lys (biscarboxymethyl)- β-Ala-Nle-c[Glu- Hyp-D- Phe-Arg-Trp-Lys]—NH₂ | 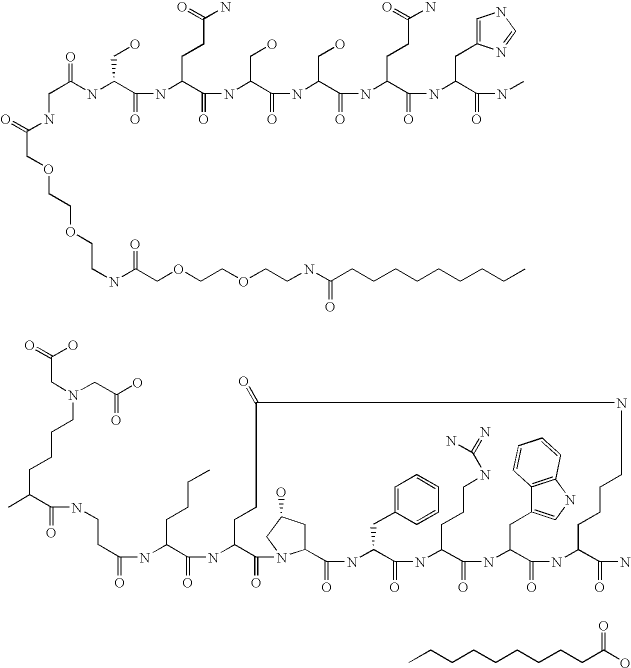 | 59 | 76 |

Assay (II)—Melanocortin Receptor 3 and 5 (MC3 and MC5) cAMP Functional Assay Using the AlphaScreen™ cAMP Detection Kit

The cAMP assays for MC3 and MC5 receptors are performed on cells (either HEK293 or BHK cells) stably expressing the MC3 and MC5 receptors, respectively. The receptors are cloned from cDNA by PCR and inserted into the pcDNA 3 expression vector. Stable clones are selected using 1 mg/ml G418.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer (5 mM HEPES, 0.1% ovalbumin, 0.005% Tween™ 20 and 0.5 mM IBMX, pH 7.4), and then resuspended in stimulation buffer to a final concentration of 1×10⁶ or 2×10⁶ cells/ml. 25 μl of cell suspension is added to the microtiter plates containing 25 μl of test compound or reference compound (all diluted in stimulation buffer). The plates are incubated for 30 minutes at room temperature (RT) on a plate-shaker set to a low rate of shaking. The reaction is stopped by adding 25 μl of acceptor beads with anti-cAMP, and 2 min later 50 μl of donor beads per well with biotinylated cAMP in a lysis buffer. The plates are then sealed with plastic, shaken for 30 minutes and allowed to stand overnight, after which they are counted in an Alpha™ microplate reader.

EC₅₀ values are calculated by non-linear regression analysis of dose/response curves (6 points minimum) using the Windows™ program GraphPad™ Prism (GraphPad™ Software, USA). All results are expressed in nM.

For measuring antagonistic activity in the MC3 functional cAMP assay, the MC3 receptors are stimulated with 3 nM α-MSH, and inhibited by increasing the amount of potential antagonist. The IC₅₀ value for the antagonist is defined as the concentration that inhibits MC3 stimulation by 50%.

Assay (III)—Melanocortin Receptor 4 (MC4) cAMP Assay

BHK cells expressing the MC4 receptor are stimulated with potential MC4 agonists, and the degree of stimulation of cAMP is measured using the Flash Plate® cAMP assay (NEN™ Life Science Products, cat. No. SMP004).

The MC4 receptor-expressing BHK cells are produced by transfecting the cDNA encoding MC4 receptor into BHK570/KZ10-20-48, and selecting for stable clones expressing the MC4 receptor. The MC4 receptor cDNA, as well as a CHO cell line expressing the MC4 receptor, may be purchased from Euroscreen™. The cells are grown in DMEM, 10% FCS, 1 mg/ml G418, 250 nM MTX and 1% penicillin/streptomycin.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and resuspended in stimulation buffer to a final concentration of 2×10⁶ cells/ml (consumption thereof: 7 ml per 96-well microtiter plate). 50 μl of cell suspension is added to the Flash Plate containing 50 μl of test compound or reference compound (all diluted in PBS, 0.1% HSA and 0.005% Tween). The mixture is shaken for 5 minutes and then allowed to stand for 25 minutes at RT. The reaction is stopped by addition of 100 μl Detection Mix per well (Detection Mix 11 ml Detection Buffer+100 μl (˜2 μCi) cAMP [¹²⁵I] tracer). The plates are then sealed with plastic, shaken for 30 minutes, and allowed to stand overnight (or for 2 hours) and then counted in the Topcounter (2 min/well). The assay procedure and the buffers are generally as described in the Flash Plate kit-protocol (Flash Plate® cAMP assay (NEN™ Life Science Products, cat. No. SMP004)). However the cAMP standards are diluted in PBS with 0.1% HSA and 0.005% Tween 20 and not in stimulation buffer.

EC₅₀ values are calculated by non-linear regression analysis of dose/response curves (6 points minimum) using the Windows™ program GraphPad™ Prism (GraphPad Software, USA). All results are expressed in nM.

### Assay (IV)—Melanocortin Receptor 1 (MC1) Binding Assay

The MC1 receptor binding assay is performed on BHK cell membranes stably expressing the MC1 receptor. The assay is performed in a total volume of 250 NI: 25 μl of ¹²⁵NDP-α-MSH (22 pM in final concentration), 25 μl of test compound/control and 200 μl of cell membrane (25 μg/ml). Test compounds are dissolved in DMSO. Radioactively labeled ligand, membranes and test compounds are diluted in buffer: 25 mM HEPES, pH 7.4, 0.1 mM CaCl₂, 1 mM MgSO₄, 1 mM EDTA, 0.1% HSA and 0.005% Tween™ 20. Alternatively, HSA may be substituted with ovalbumin. The samples are incubated at 30° C. for 90 min. in Costar round-bottom microtiter plates. Incubation is terminated by filtration on a Packard harvester filtermate. Rapid filtration through Packard Unifilter-96 GF/B filters pre-treated with polyethylenimine (PerkinElmer 6005277). The filters are washed with ice-cold 0.9% NaCl 8-10 times. The plates is air dried at app. 55° C. for 30 min, and 50 μl Microscint 0 (Packard, cat. No. 6013616) is added to each well. The plates are counted in a Topcounter (1 min/well).

The data are analysed by non-linear regression analysis of binding curves, using the Windows™ program GraphPad™ Prism (GraphPad Software, USA).

TABLE 3
In vitro data on receptor binding

| | | | Assay | Assay |
|---|---|---|---|---|
| | | | (V) | (IV) |
| | | | MC4 | MC1 |
| Exam- | Com- | | [Ki] | Ki |
| ple nr. | pound | Molecule | (nM) | (nM) |
| Exam- ple 1 | (2-{2-[16- (Tetrazol- 5- yl)hexa- decanoyl- amino] ethoxy} ethoxy) acetyl- Gly-Ser- Gln-His- Lys (BCMA)- Nle-c[Glu- Hyp-D- Phe-Arg- Trp- Lys]—NH₂ | 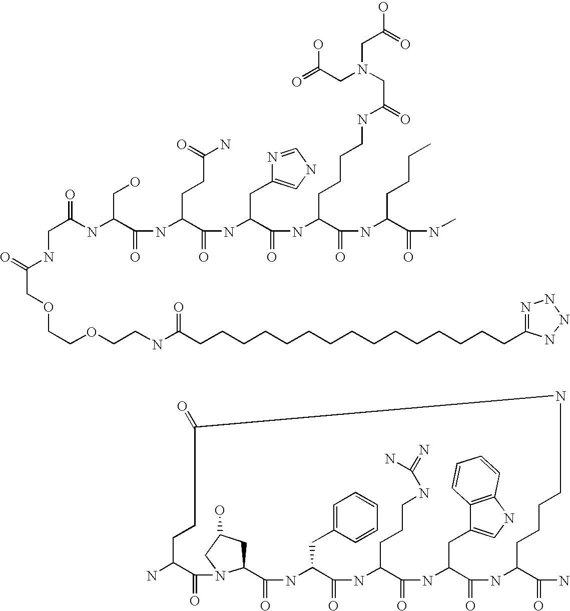 | 0.4 | >10000 |
| Exam- ple 2 | (2-{2-[16- (Tetrazol- 5- yl)hexa- decanoyl- amino] ethoxy} ethoxy) acetyl- Gly-Ser- Gln-His- Dap (BCMA)- Nle-c[Glu- Hyp-D- Phe-Arg- Trp- Lys]—NH₂ | 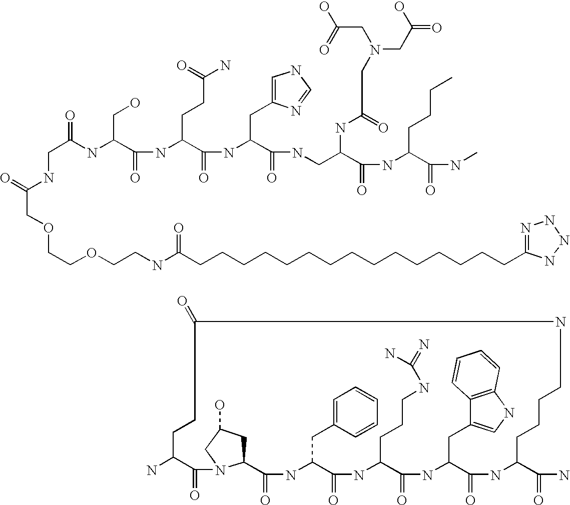 | 3.43 | 5567 |
| Exam- ple 3 | (2-{2-[2- (2-{2- [16- (Tetrazol- 5-yl) hexa- decanoyl- amino] ethoxy} ethoxy) acetyl- amino] ethoxy} ethoxy) acetyl- Gly-D- Ser-Gln- Ser-Ser- Gln-His- Lys(bis- carboxy- methyl)- Nle-c[Glu- Hyp-D- Phe-Arg- Trp- Lys]— NH₂ | 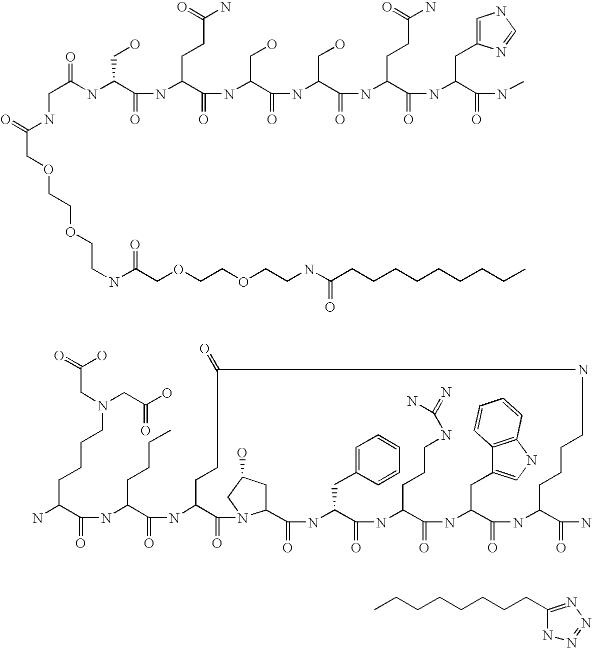 | 1.95 | >10000 |
| Exam- ple 4 | (2-{2-[16- (Tetrazol- 5-yl) hexa- decanoyl- amino] ethoxy} ethoxy) acetyl- Gly-Ser- Gln-Ser- Dap (BCMA)- Nle-c[Glu- Hyp-D- Phe-Arg- Trp- Lys]— NH₂ | 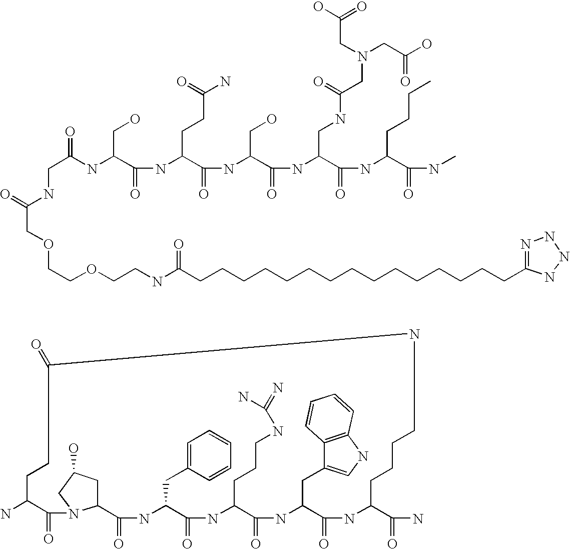 | 5.67 | 4796 |
| Exam- ple 5 | (2-{2-[16- (Tetrazol- 5-yl) hexa- decanoyl- amino] ethoxy} ethoxy) acetyl- Gly-Ser- Gln-Ser- Lys(bis- carboxy- methyl)- Nle-c[Glu- Hyp-D- Phe-Arg- Trp- Lys]—NH₂ | 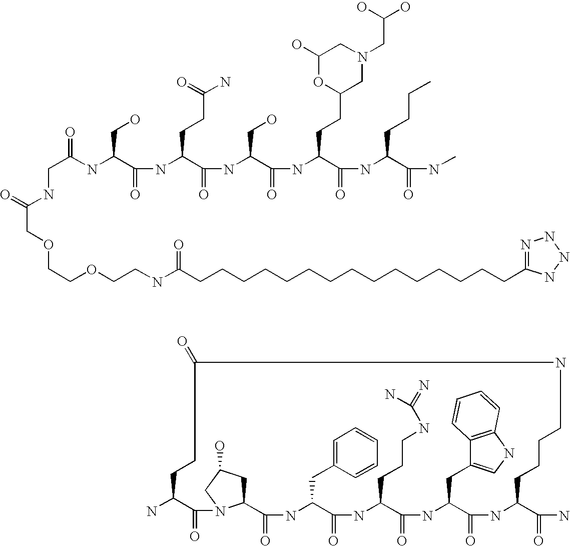 | 7.55 | >10000 |
| Exam- ple 6 | (2-{2-[16- (Tetrazol- 5-yl) hexa- decanoyl- amino] ethoxy} ethoxy) acetyl- Gly-Ser- Gln-His- Dap(bis- carboxy- methyl)- Nle-c[Glu- Hyp-D- Phe-Arg- Trp- Lys]—NH₂ | 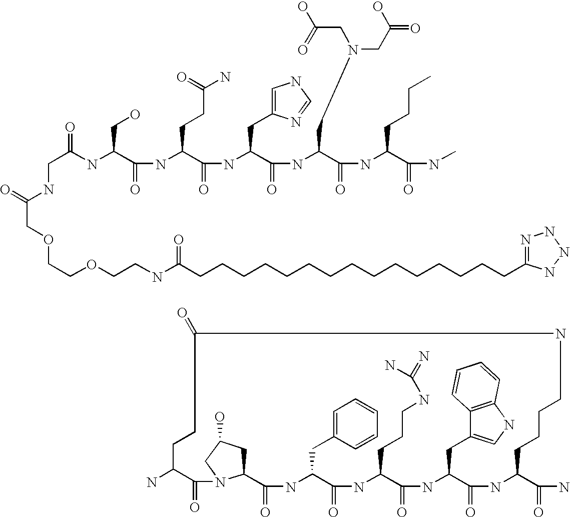 | 2.9 | 5860 |
| Exam- ple 7 | (2-{2-[16- (Tetrazol- 5-yl) hexa- decanoyl- amino] ethoxy} ethoxy) acetyl- Gly-Ser- Gln-His- Lys(bis- carboxy- methyl)- Nle-c[Glu- Hyp-D- Phe-Arg- Trp- Lys]—NH₂ | 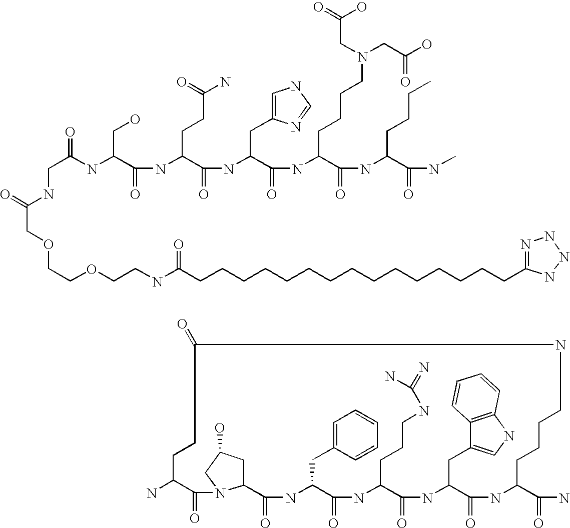 | 1.6 | 8712 |
| Exam- ple 8 | (2-{2-[16- (Tetrazol- 5-yl) hexa- decanoyl- amino] ethoxy} ethoxy) acetyl- Gly-Ser- Gln-His- Orn (BCMA)- Nle-c[Glu- Hyp-D- Phe-Arg- Trp- Lys]—NH₂ | 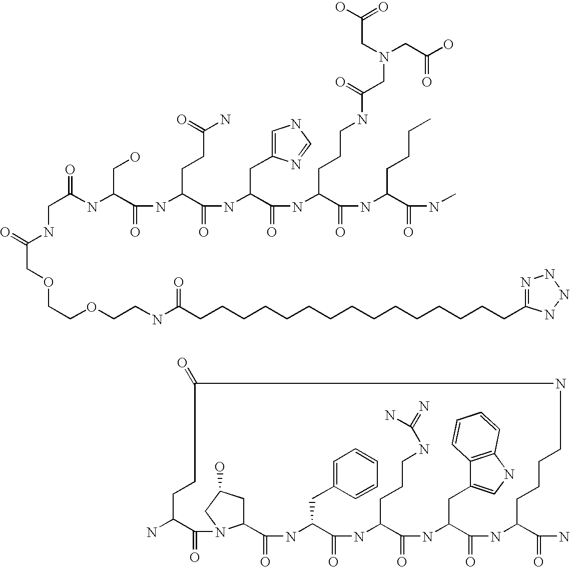 | 0.9 | >10000 |
| Exam- ple 9 | (2-{2-[16- (Tetrazol- 5-yl) hexa- decanoyl- amino] ethoxy} ethoxy) acetyl- Gly-Ser- Gln-His- Dap (BCMA)- Nle-c[Glu- Hyp-D- Phe-Arg- Trp- Lys]—NH₂ | 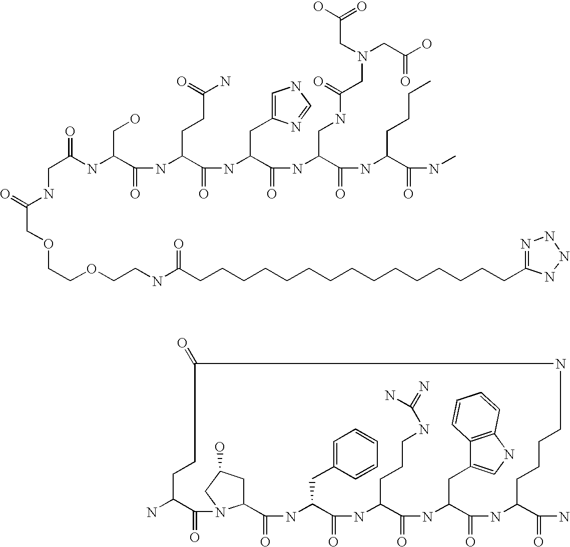 | 0.58 | 2673 |
| Exam- ple 10 | (2-{2-[16- (Tetrazol- 5-yl) hexa- decanoyl- amino] ethoxy} ethoxy) acetyl- Gly-Ser- Gln-His- Dab (BCMA)- Nle-c[Glu- Hyp-D- Phe-Arg- Trp- Lys]—NH₂ | 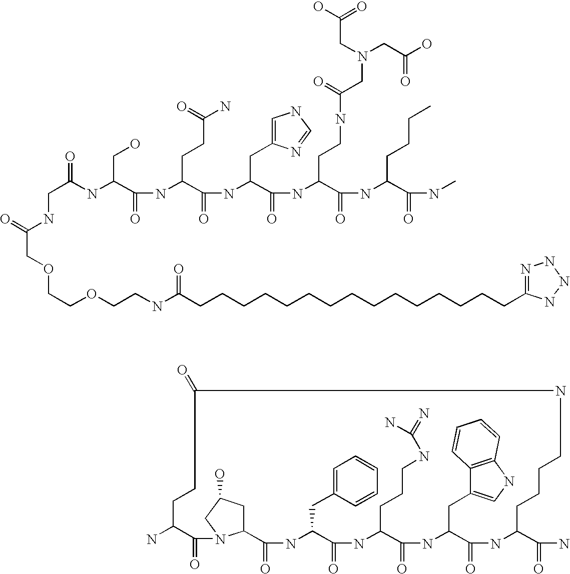 | 1.05 | >10000 |
| Exam- ple 11 | [2-(2-{4- [16- (Tetrazol- 5-yl) hexa- decanoyl- sulfamoyl] butanoyl- amino} ethoxy) ethoxy] acetyl- Gly-Ser- Gln-His- Dap (BCMA)- Nle-c[Glu- Hyp-D- Phe-Arg- Trp- Lys]—NH₂ | 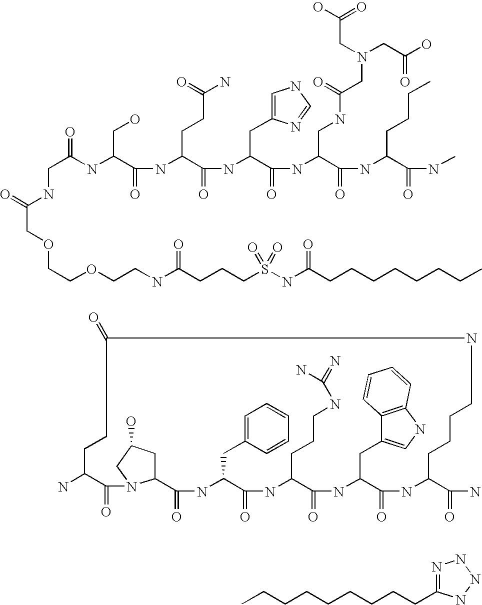 | 3.25 | 9698 |
| Exam- ple 12 | (2-{2-[2- (2-{2- [16- (Tetrazol- 5-yl) hexa- decanoyl- amino] ethoxy} ethoxy) acetyl- amino] ethoxy} ethoxy) acetyl- Gly-Ser- Gln-Ser- Ser-Gln- Lys(bis- carboxy- methyl)- Nle-c[Glu- Hyp-D- Phe-Arg- Trp- Lys]—NH₂ | 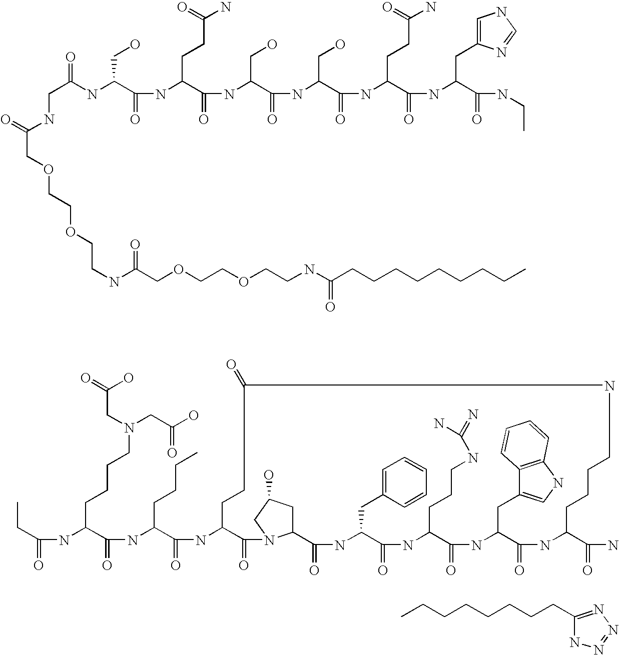 | 2.85 | >10000 |
| Exam- ple 13 | {2-(2-(15- Carboxy- pentade- canoyl- amino) ethoxy] ethoxy} acetyl- Gly-Ser- Gln-His- Dap (BCMA)- Nle-c[Glu- Hyp-D- Phe-Arg- Trp- Lys]—NH₂ | 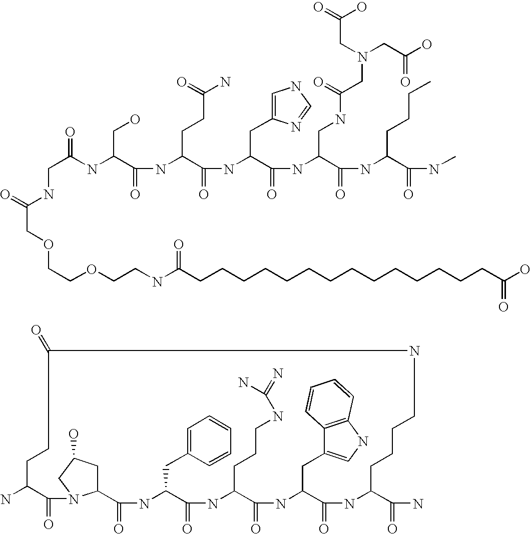 | 3.6 | >10000 |
| Exam- ple 14 | (2-{2-{2- (2-{2-[16- (Tetrazol- 5-yl) hexa- decanoyl- amino] ethoxy} ethoxy) acetyl- amino] ethoxy} ethoxy) acetyl- amino] ethoxy} ethoxy) acetyl- amino] ethoxy} ethoxy) acetyl- Gly-Ser- Gln-Ser- Dap (BCMA)- Nle-c[Glu- Hyp-D- Phe-Arg- Trp- Lys]—NH₂ | 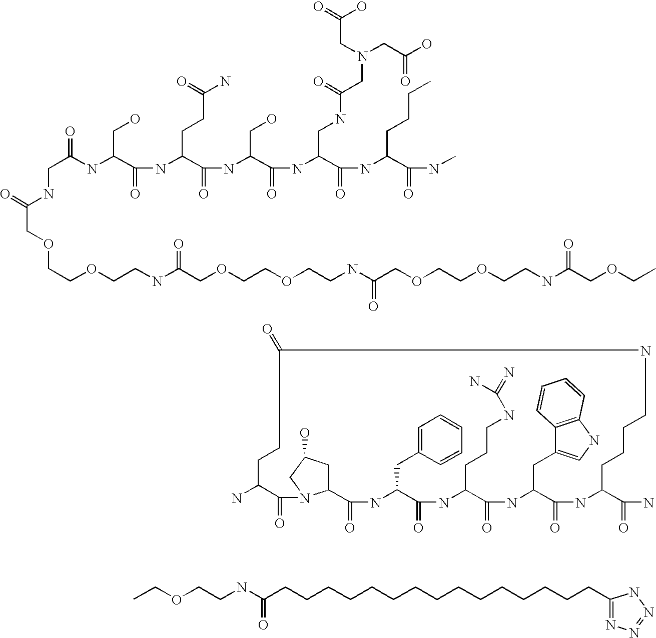 | 8.65 | >10000 |
| Exam- ple 15 | (2-{2-[16- (Tetrazol- 5- yl)hexa- decanoyl- amino] ethoxy} ethoxy) acetyl- Gly-Ser- Gln-Ser- Lys (BCMA)- Nle-c[Glu- Hyp-D- Phe-Arg- Trp- Lys]—NH₂ | 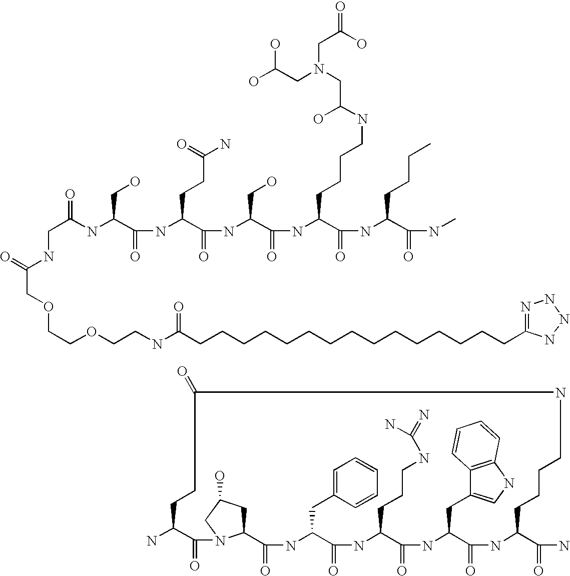 | 3.15 | >10000 |
| Exam- ple 16 | (2-{2-[2- (2-{2-[2- (2-{2-[2- (2-{2-[16- (Tetrazol- 5-yl)hexa- decanoyl- amino] ethoxy} ethoxy) acetyl- amino] ethoxy} ethoxy) acetyl- amino] ethoxy} ethoxy) acetyl- amino] ethoxy} ethoxy) acetyl- Gly-Ser- Gln-His- Dap (BCMA)- Nle-c[Glu- Hyp-D- Phe-Arg- Trp- Lys]—NH₂ | 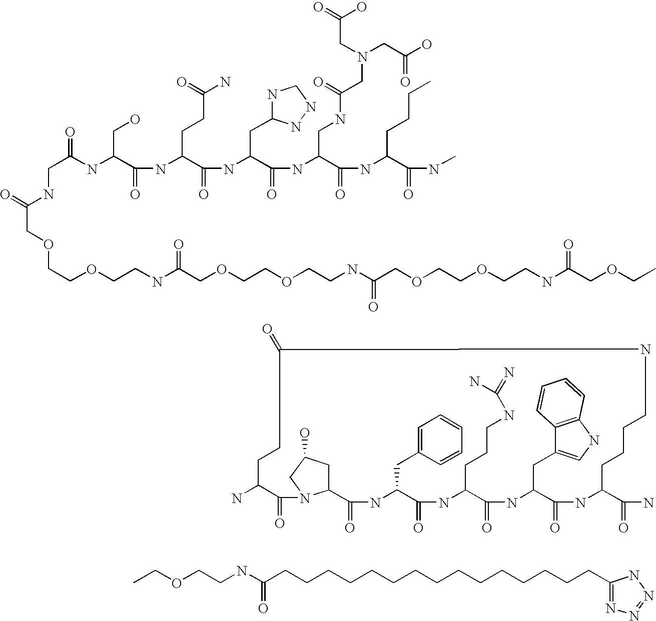 | 2.75 | >10000 |
| Exam- ple 17 | (2-{2-[16- (Tetrazol- 5-yl)hexa- decanoyl- amino] ethoxy} ethoxy) acetyl- Glu-Ser- Gln-His- Dap (BCMA)- Nle-c[Glu- Hyp-D- Phe-Arg- Trp- Lys]—NH₂ | 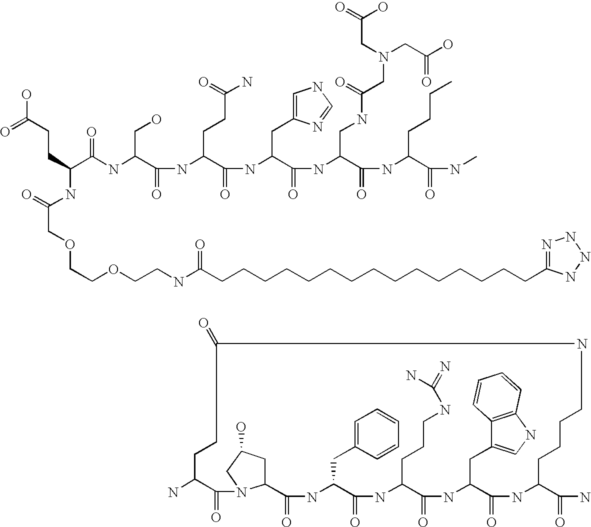 | 5.4 | >10000 |
| Exam- ple 18 | (2-{2- [2-(2-{2- [(S)-4- Carboxy- 4-(17- carboxy- heptade- canoyl- amino) butanoyl- amino] ethoxy} ethoxy) acetyl- amino] ethoxy} ethoxy) acetyl- Gly-Ser- Gln-His- Dap (BCMA)- Nle-c[Glu- Hyp-D- Phe-Arg- Trp- Lys]— NH₂ | 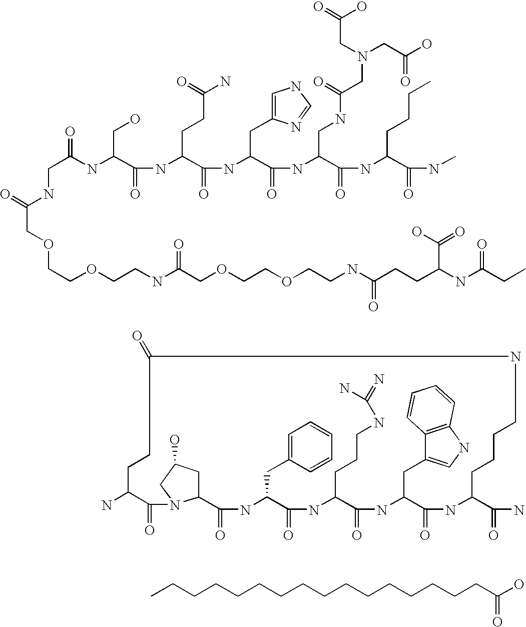 | 5.35 | >10000 |
| Exam- ple 19 | (2-{2-[16- (Tetrazol- 5-yl)hexa- decanoyl- amino] ethoxy} ethoxy) acetyl- Gly-Ser- Gln-Tyr- Dap (BCMA)- Nle-c[Glu- Hyp-D- Phe-Arg- Trp- Lys]—NH₂ | 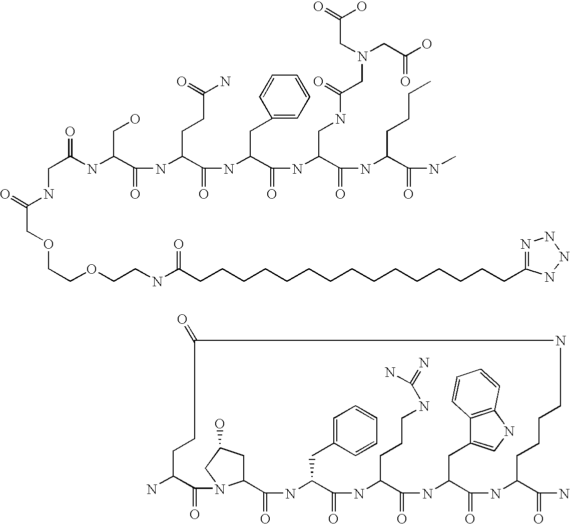 | 3.9 | >10000 |
| Exam- ple 20 | (2-{2- (2-(2-{2- [16- (Tetrazol- 5-yl)hexa- decanoyl- amino] ethoxy} ethoxy) acetyl- amino] ethoxy} ethoxy) acetyl- Gly-D- Ser-Gln- Ser-Ser- Gln-His- β-Ala- Lys(bis- carboxy- methyl)- Nle-c[Glu- Hyp-D- Phe-Arg- Trp- Lys]—NH₂ | 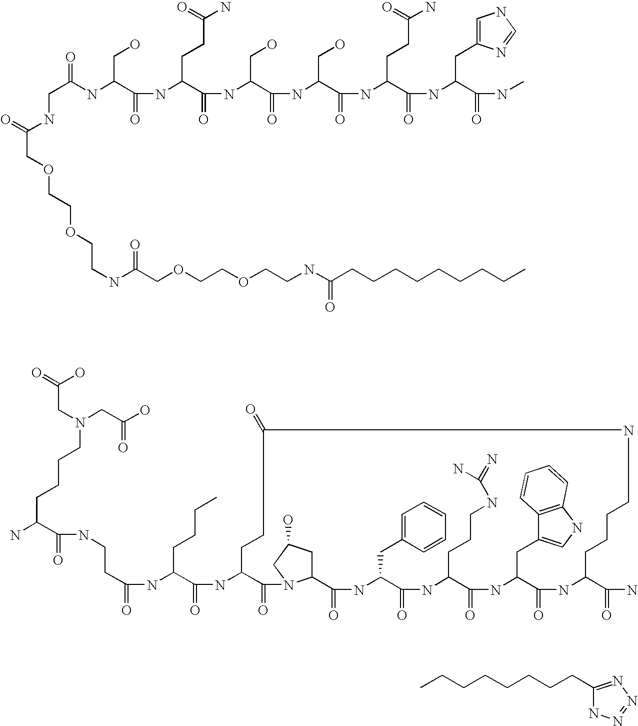 | 4.55 | >10000 |
| Exam- ple 21 | {2-[2-(15- Carboxy- penta- decanoyl- amino) ethoxy] ethoxy} acetyl- Gly-Ser- Gln-Ser- Lys (BCMA)- Nle-c[Glu- Hyp-D- Phe-Arg- Trp- Lys]—NH₂ | 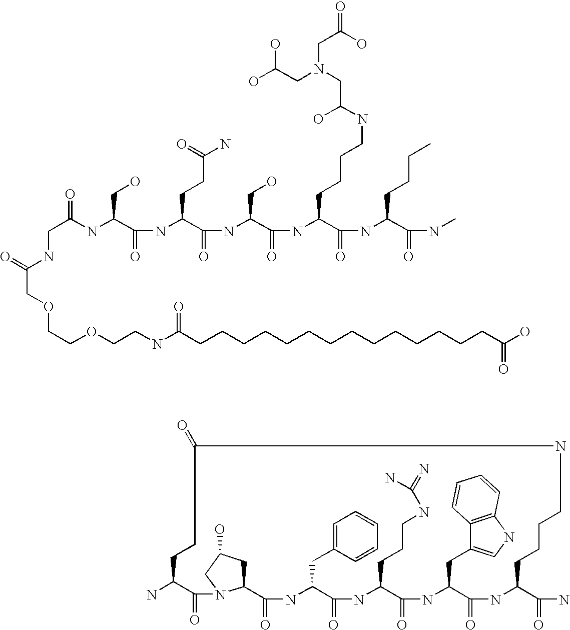 | 14.7 | >10000 |
| Exam- ple 22 | {2-[2- (2-{2-[2- (19- Carboxy- non- adecanoyl- amino) ethoxy] ethoxy} acetyl- amino) ethoxy] ethoxy} acetyl- Gly-D- Ser-Gln- Ser-Ser- Gln-His- Lys(bis- carboxy- methyl)- β-Ala- Nle-c[Glu- Hyp-D- Phe-Arg- Trp- Lys]—NH₂ | 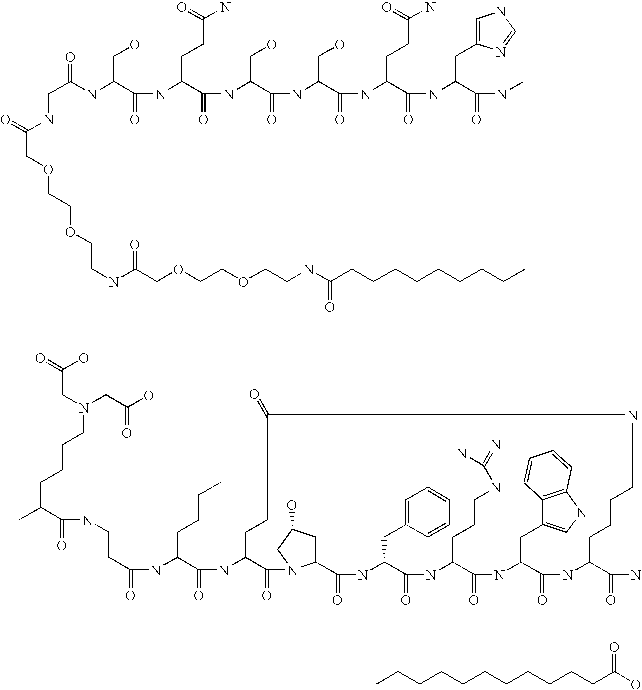 | 0.55 | >10000 |
| Exam- ple 23 | (2-{2-[16- (Tetrazol- 5-yl)hexa- decanoyl- amino] ethoxy} ethoxy) acetyl- Gly-Ser- Gln-Tyr- Dap(bis- carboxy- methyl)- Nle-c[Glu- Hyp-D- Phe-Arg- Trp- Lys]—NH₂ | 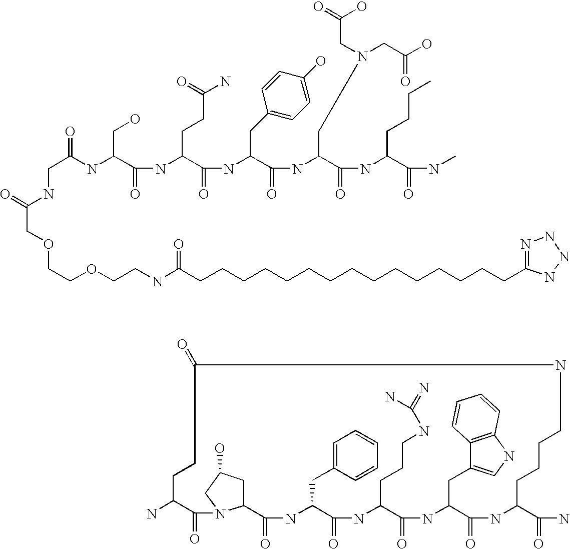 | 7.9 | >10000 |

### Assay (V)—Melanocortin Receptor 4 (MC4) Binding Assay

In vitro ¹²⁵NDP-α-MSH binding to recombinant BHK cells expressing human MC4 receptor (filtration assay).

The assay is performed in 5 ml minisorb vials (Sarstedt No. 55.526) or in 96-well filterplates (Millipore MADVN 6550), and using BHK cells expressing the human MC4 receptor using BHK cells stably expressing the human MC4 receptor. The membranes were prepared from frozen or fresh cells that were homogenized in 20 mM HEPES pH 7.1, 5 mM MgCl₂ and 1 mg/ml bacitracin and centrifuged at 15000 rpm at 4° C., 10 min in a Sorvall RC 5B plus, SS-34 rotor. The supernatant was discarded, and the pellets were re-suspended in buffer, homogenized and centrifuged two more times. The final pellets were resuspended in the buffer mentioned above, and the protein concentration was measured and adjusted with buffer to 14 to 17 mg/ml and the membrane preparation were kept at −80° C. until assay. The assay was run directly on a dilution of this cell membrane suspension, without any further preparation. The BHK cell membranes are kept at −80° C. until assay, and the assay is run directly on a dilution of this cell membrane suspension, without further preparation. The suspension is diluted to give maximally 10% specific binding, i.e. to approx. 50-100 fold dilution. The assay is performed in a total volume of 200 μl: 50 μl of cell suspension, 50 μl of ¹²⁵NDP-α-MSH (≈79 pM in final concentration), 50 μl of test compound and 50 μl binding buffer (pH 7) mixed and incubated for 2 h at 25° C. [binding buffer: 25 mM HEPES, pH 7.0, 1 mM CaCl₂, 1 mM MgSO₄, 1 mM EGTA, 0.02% Bacitracin, 0.005% Tween™ 20 and 0.1% HSA or, alternatively, 0.1% ovalbumin (Sigma; catalogue No. A-5503)]. Test compounds are dissolved in DMSO and diluted in binding buffer. Radiolabelled ligand and membranes are diluted in binding buffer. The incubation is stopped by dilution with 2×100 μl ice-cold 0.9% NaCl. The radioactivity retained on the filters is counted using a Cobra II auto gamma counter.

The data are analysed by non-linear regression analysis of binding curves, using the Windows™ program GraphPad™ Prism (GraphPad Software, USA).

### Assay (VI)—Evaluation of Energy Expenditure

TAC:SPRD rats or Wistar rats from M&B Breeding and Research Centre A/S, Denmark are used. After at least one week of acclimatization, rats are placed individually in metabolic chambers (Oxymax system, Columbus Instruments, Columbus, Ohio, USA; systems calibrated daily). During the measurements, animals have free access to water, but no food is provided to the chambers. Light:dark cycle is 12 h:12 h, with lights being switched on at 6:00. After the animals have spent approx. 2 hours in the chambers (i.e. when the baseline energy expenditure is reached), test compound or vehicle are administered (po, ip or sc), and recording is continued in order to establish the action time of the test compound. Data for each animal (oxygen consumption, carbon dioxide production and flow rate) are collected every 10-18 min for a total of 22 hours (2 hours of adaptation (baseline) and 20 hours of measurement). Correction for changes in O₂ and CO₂ content in the inflowing air is made in each 10-18 min cycle.

Data are calculated per metabolic weight [(kg body weight)^{0.75}] for oxygen consumption and carbon dioxide production, and per animal for heat. Oxygen consumption (VO₂) is regarded as the major energy expenditure parameter of interest.

### Assay (VII)—Evaluation of Binding to Albumin

Test compounds are tested in a functional assay (Assay III) and a binding assay (Assay V), wherein Assay III contains HSA, and Assay V contains ovalbumin. EC₅₀ values are determined from Assay III, and Ki values from Assay V. The ratio EC₅₀/Ki is then calculated.

In the event of no albumin binding the ratio EC₅₀/Ki will be 1 or below. The stronger the binding to albumin, the higher will be the ratio; for albumin-binding test compounds, the ratio EC₅₀/Ki will thus be ≧1, such as ≧10, e.g. ≧100.

### Assay (VIII)—Melanocortin Receptor 3 (MC3) Binding Assay

The MC3 receptor binding assay is performed on BHK cell membranes stably expressing the human MC3 receptor. The human MC3 receptor is cloned by PCR and subcloned into pcDNA3 expression vector. Cells stably expressing the human MC3 receptor are generated by transfecting the expression vector into BHK cells and using G418 to select for MC3 clones. The BHK MC3 clones are cultured in DMEM with glutamax, 10% FCS, 1% pen/strep and 1 mg/ml G418 at 37° C. and 5% CO₂.

The binding is performed on a membrane preparation prepared in the following way: The cells are rinsed with PBS and incubated with Versene for approximately 5 min before harvesting. The cells are flushed with PBS and the cell-suspension is centrifuged for 10 min at 2800×G. The pellet is resuspended in 20 ml buffer (20 mM Tris pH 7.2+5 mM EDTA+1 mg/ml Bacitracin (Sigma B-0125)) and homogenized with a glass-teflon homogenizer, 10 times and low speed. The cell suspension is centrifuged at 4° C., 4100×G for 20 min. Pellet is resuspended in buffer and the membranes are diluted to a protein concentration of 1 mg/ml in buffer, aliquoted and kept at −80° C. until use.

The assay is performed in a volume of 100 μl. Mix in the following order 25 μl test compound, 25 μl ¹²⁵I-NDP-α-MSH (app. 60 000 cpm/well˜0.25 nM in final concentration) and 50 μl membranes (30 μg/well) and incubate in Costar round-bottom wells microtiter plate, (catalogue number 3365). Test-compounds are dissolved in DMSO or H₂O. Radioligand, membranes and test compounds are diluted in buffer; (25 mM HEPES pH 7.4, 1 mM CaCl2, 5 mM MgSO4, 0.1% Ovalbumin (Sigma A-5503), 0.005% Tween-20 and 5% Hydroxypropyl-β-cyclodextrin 97%, (Acros organics, code 297561000). The assay mixture is incubated for 1 h at 20-25° C. Incubation is terminated by filtration on a Packard harvester filtermate 196. Rapid filtration through Packard Unifilter-96 GF/B filters pre-treated for 1 h with 0.5% polyethylenimine is carried out. The filters are washed with ice-cold 0.9% NaCl 8-10 times. The plate is air dried at 55° C. for 30 min, and 50 μl Microscint 0 (Packard) is added. The radioactivity retained on the filter is counted using a Packard TopCount.NXT.

Results; IC₅₀ values are calculated by non-linear regression analysis of binding curves (6 points minimum) using the windows program GraphPad Prism, GraphPad software, USA. Ki-values were calculated according to the Cheng-Prusoff equation [Y-C. Cheng and W. H. Prusoff, Biochem. Pharmacol. 22 (1973) pp. 3099-3108].

### Assay (IX)—Melanocortin Receptor 5 (MC5) Binding Assay

The MC5 receptor binding assay is performed on BHK cell membranes stably expressing the human MC3 receptor. The human MC5 receptor is cloned by PCR and subcloned into pcDNA3 expression vector. Cells stably expressing the human MC5 receptor are generated by transfecting the expression vector into BHK cells and using G418 to select for MC5 clones. The BHK MC5 clones are cultured in DMEM with glutamax, 10% FCS, 1% pen/strep and 1 mg/ml G418 at 37° C. and 5% CO₂.

The binding is performed on a membrane preparation prepared in the following way: The cells are rinsed with PBS and incubated with Versene for approximately 5 min before harvesting. The cells are flushed with PBS and the cell suspension is centrifuged for 10 min at 2800×G. The pellet is resuspended in 20 ml buffer (20 mM Tris pH 7.2+5 mM EDTA+1 mg/ml Bacitracin (Sigma B-0125)) and homogenized with a glass-teflon homogenizer, 10 times and low speed. The cell-suspension is centrifuged at 4° C., 4100×G for 20 min. Pellet is resuspended in buffer and the membranes are diluted to a protein concentration of 1 mg/ml in buffer, aliquoted and kept at −80° C. until use.

The assay is performed in a volume of 100 μl. Mix in the following order 25 μl test-compound, 25 μl ¹²⁵I-NDP-α-MSH (app. 60 000 cpm/well˜0.25 nM in final concentration) and 50 μl membranes (10 μg/well) and incubate incubation in Costar round-bottom wells microtiter plate, catalogue number 3365: Test-compounds are dissolved in DMSO or H₂O. Radioligand, membranes and test-compounds are diluted in buffer; (25 mM HEPES pH 7.4, 1 mM CaCl2, 5 mM MgSO4, 0.1% Ovalbumin (Sigma A-5503), 0.005% Tween-20 and 5% Hydroxypropyl-β-cyclodextrin, (97%, Acros organics, code 297561000). The assay mixture is incubated for 1 h at 20-25° C. Incubation is terminated by filtration on a Packard harvester filtermate 196. Rapid filtration through Packard Unifilter-96 GF/B filters pre-treated for 1 h with 0.5% polyethylenimine is carried out. The filters are washed with ice-cold 0.9% NaCl 8-10 times. The plate is air dried at 55° C. for 30 min, and 50 μl Microscint 0 (Packard) is added. The radioactivity retained on the filter is counted using a Packard TopCount.NXT.

Results: IC₅₀ values are calculated by non-linear regression analysis of binding curves (6 points minimum) using the windows program GraphPad Prism, GraphPad software, USA. Ki-values were calculated according to the Cheng-Prusoff equation [Y-C. Cheng and W. H. Prusoff, Biochem. Pharmacol. 22 (1973) pp. 3099-3108].
Assay (X)—Melanocortin Receptor 3 (MC3) cAMP Functional Assay Using The FlashPlate® cAMP Detection Kit

The MC3-containing BHK cells are stimulated with potential MC3 agonists, and the degree of stimulation of CAMP is measured using the FlashPlate® cAMP assay, cat. No SMP004, NEN™ Life Science Products.

### BHK/hMC3 Clone 5 Cells

The cells are produced by transfecting the cDNA encoding MC3 receptor into BHK570, and selecting for stable clones expressing the hMC3 receptor. The cells are grown in DMEM, 10% FCS, 1 mg/ml G418 and 1% pen/strep.

Cells at approx. 80-90% confluence are washed with PBS, lifted from the plates with Versene and diluted in PBS. After centrifugation for 5 min at 1300 rpm the supernatant is removed, and the cells are resuspended in stimulation buffer to a final concentration of 2×10⁶ cells/ml. 50 μl cell suspension is added to the Flashplate containing 50 μl of test-compound or reference compound (all dissolved in DMSO and diluted in 0.1% HSA (Sigma A-1887) and 0.005% Tween 20). The mixture is shaken for 5 minutes and then allowed to stand for 25 minutes at room temperature. The reaction is stopped with 100 μl Detection Mix pro well (Detection Mix 11 ml Detection Buffer+100 μl (˜2 μCi) cAMP [¹²⁵I] Tracer). The plates are then sealed with plastic, shaken for 30 minutes and allowed to stand overnight (or for 2 h), and then counted in the Topcounter, 2 min/well (Note that in general, the assay procedure described in the kit-protocol is followed; however, the cAMP standards are diluted in 0.1% HSA and 0.005% Tween 20, and not in stimulation buffer).

### Results

EC₅₀ values are calculated by non-linear regression analysis of dose-response curves (6 points minimum) using the Windows program GraphPad Prism, GraphPad software, USA. Results are expressed in nM. Eₘₐₓ values are calculated as % of NDP-α-MSH maximal stimulation in the hMC3cAMP assay (maximal NDP-α-MSH stimulation 100%).

1. A compound according to formula I:
R¹—R²—C(═O)—R³—S¹—Z¹—Z²—Z³—Z⁴—Z⁵—Z⁶-c[X¹—X²—X³—-Arg-X⁴—X⁵]—Z⁷—R⁴ [I]
wherein
R¹ represents tetrazol-5-yl or carboxy;
R² represents a straight-chain, branched and/or cyclic C₆₋₂₀alkylene, C₆₋₂₀alkenylene or C₆₋₂₀alkynylene which may optionally be substituted with one or more substituents selected from halogen, hydroxy and aryl;
R³ is absent or represents —NH—S(O)₂—(CH₂)₃₋₅—C(O)— or a peptide fragment comprising one or two amino acid residues derived from natural or unnatural amino acids and containing at least one carboxy group;
wherein the side chains of R³ must not contain amino, guanidino, imidazolyl or other basic groups positively charged at neutral pH;
S¹ is absent or represents a glycolether-based structure according to one of the formulas IIa-IIh;
—HN—CH₂—CH₂—O—CH₂—CH₂—O—CH₂—C(═O)— [IIa]
—[HN—CH₂—CH₂—O—CH₂—CH₂—O—CH₂—C(═O)]₂— [IIb]
—[HN—CH₂—CH₂—O—CH₂—CH₂—O—CH₂—C(═O)]₃₋₅— [IIc]
—[HN—CH₂—CH₂—O—CH₂—CH₂—O—CH₂—CH₂—NH—C(═O)—CH₂—CH₂—CH₂—C(═O)]₁₋₃— [IId]
—[HN—CH₂—CH₂—O—CH₂—CH₂—O—CH₂—CH₂—NH—C(═O)—CH₂—O—CH₂—C(═O)]₁₋₃— [IIe]
—[HN—CH₂—CH₂—O—CH₂—CH₂—O—CH₂—CH₂—O—CH₂—CH₂—O—CH₂—CH₂—C(═O)]₁₋₃— [IIf]
—HN—CH₂—CH₂—[O—CH₂—CH₂]₂₋₁₂—O—CH₂—C(═O)— [IIg]
—HN—CH₂—CH₂—[O—CH₂—CH₂]₄₋₁₂—O—CH₂—CH₂—C(═O)— [IIg]
Z¹ is absent or represents a peptide fragment comprising one to four amino acid residues derived from natural or unnatural amino acids;
wherein the side chains of Z¹ do not contain amino, guanidino, imidazolyl or other basic groups positively charged at neutral pH;
Z² represents Gly, β-Ala, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;
Z³ represents Gly, β-Ala, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;
Z⁴ represents Gly, Ala, β-Ala, D-Ala, Pro, D-Pro, Hyp, D-Hyp, Ser, D-Ser, homoSer, D-homoSer, Thr, D-Thr, Tyr, D-Tyr, Phe, D-Phe, Gln, D-Gln, Asn, D-Asn, 2-PyAla, D-2-PyAla, 3-PyAla, D-3-PyAla, 4-PyAla, D-4-PyAla, His or D-His;
with the proviso that not more than one of residues Z², Z³ and Z⁴ is His or D-His;
Z⁵ represents a structure according to one of the formulas IIIa, IVa, Va, VIIa, VIIa, VIIIa, IXa, Xa, IIIb, IVb, Vb, VIb, VIIb, VIIIb, IXb, or Xb; 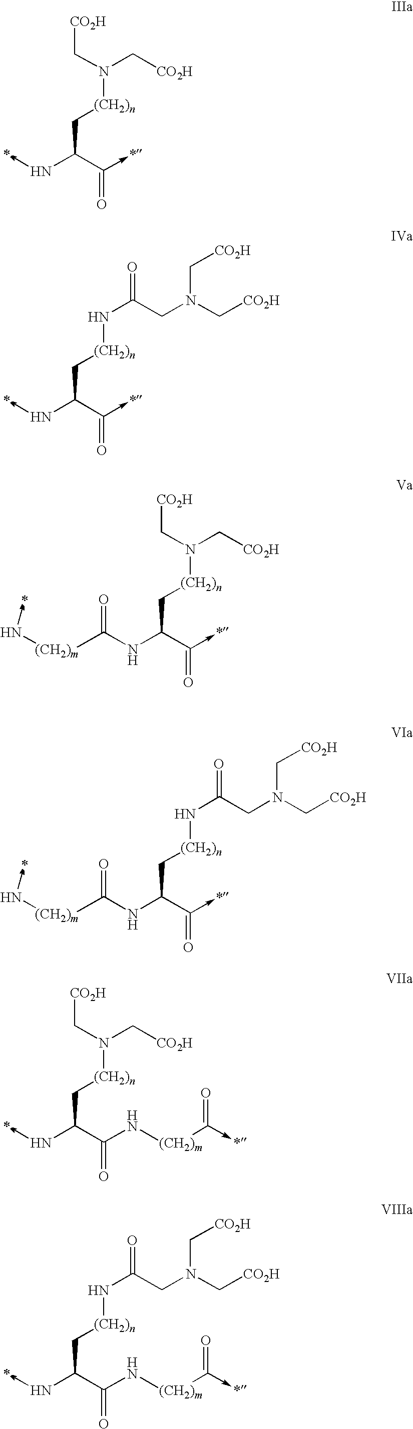 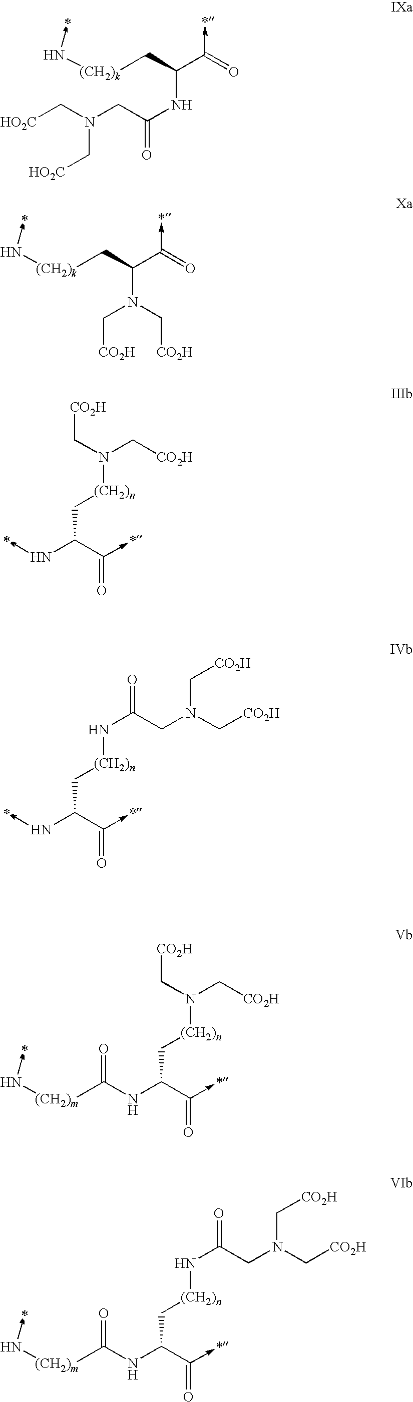 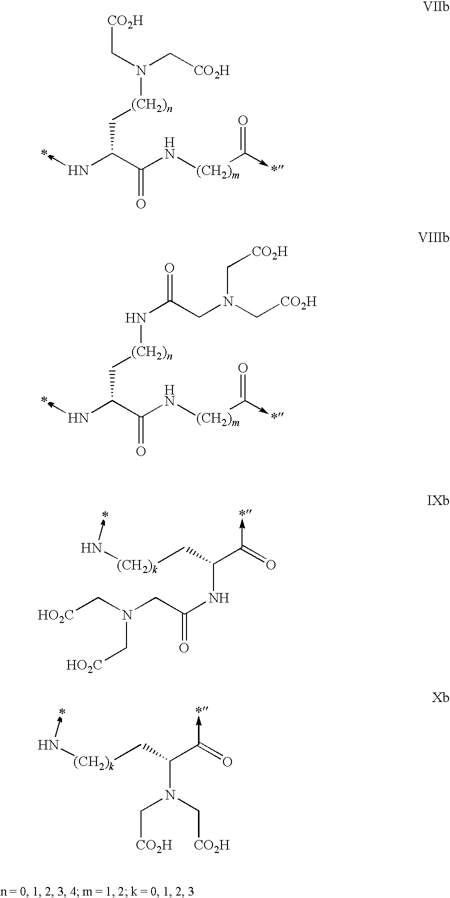
wherein n in formulas Ma to VIIIa and Mb to VIIIb is 0, 1, 2, 3 or 4,
m in formulas Va to VIIIa and Vb to VIIIb is 1 or 2,
k in formulas IXa, Xa, IXb and Xb is 0, 1, 2 or 3;
Z⁶ in formula I represents Ala, D-Ala, Val, D-Val, Leu, D-Leu, Ile, D-Ile, Met, D-Met, Nle, D-Nle, Phe, D-Phe, Tyr, D-Tyr, Trp or D-Trp;
X¹ represents Glu, Asp, Cys, homoCys, Lys, Orn, Dab or Dap;
X² represents His, Cit, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, Aze, Pip, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein one or more hydrogens on the phenyl moiety of said Phe may optionally and independently be substituted by a substituent selected among halogen, hydroxy, alkoxy, nitro, benzoyl, methyl, trifluoromethyl and cyano;
X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;
X⁴ represents Trp, 2-Nal, (3-benzo[b]thienyl)alanine or (S)-2,3,4,9-tetrahydro-1H-β-carboline-3-carboxylic acid;
X⁵ represents Glu, Asp, Cys, homoCys, Lys, Orn, Dab or Dap;
wherein X¹ and X⁵ are joined, rendering the compound of formula I cyclic, either via a disulfide bridge deriving from X¹ and X⁵ both independently being Cys or homoCys, or via an amide bond formed between a carboxylic acid in the side-chain of X¹ and an amino group in the side-chain of X⁵, or between a carboxylic acid in the side-chain of X⁵ and an amino group in the side-chain of X¹;
Z⁷ is absent or represents a peptide fragment comprising one to three amino acid residues derived from natural or unnatural amino acids;
wherein the side chains of Z⁷ do not contain amino, guanidino, imidazolyl or other basic groups positively charged at neutral pH;
R⁴ represents OR′ or N(R′)₂, wherein each R′ independently represents hydrogen or represents C₁₋₆alkyl, C₂₋₆alkenyl or C₂₋₆alkynyl which may optionally be substituted with one or more hydroxy;
and pharmaceutically acceptable salts, prodrugs and solvates thereof.

2. A compound according to claim 1, selected from the group consisting of:
(2-{2-[16-(Tetrazol-5-yl)hexadecanoylamino]ethoxy}ethoxy)acetyl-Gly-Ser-Gln-His-Lys(BCMA)-Nle-c[Glu-Hyp-D-Phe-Arg-Trp-Lys]-NH₂ 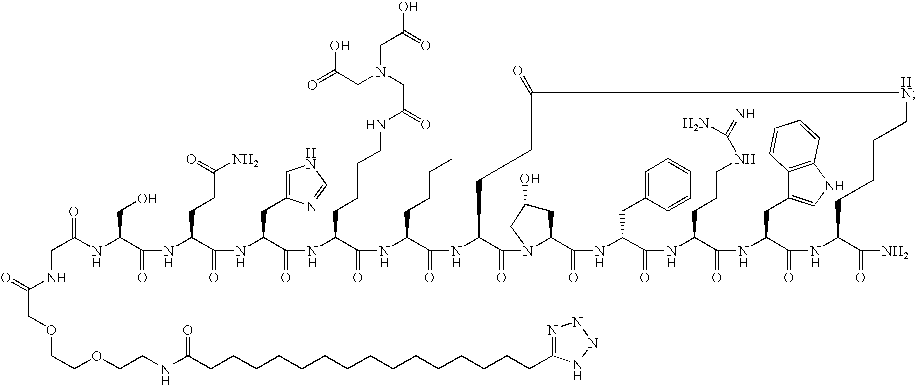
(2-{2-[16-(Tetrazol-5-yl)hexadecanoylamino]ethoxy}ethoxy)acetyl-Gly-Ser-Gln-His-β-Dap(BCMA)-Nle-c[Glu-Hyp-D-Phe-Arg-Trp-Lys]-NH₂ 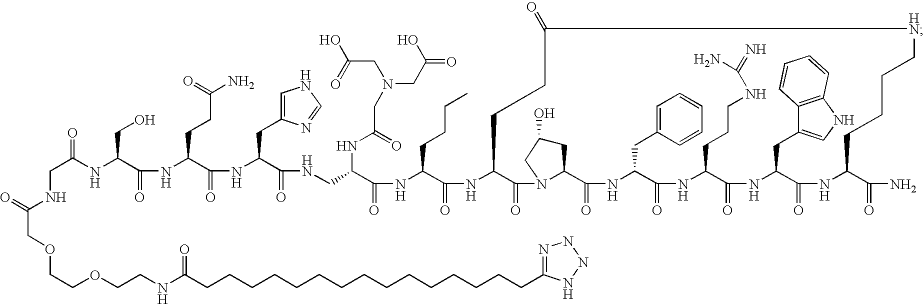
(2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-Lys(biscarboxymethyl)-Nle-c[Glu-Hyp-D-Phe-Arg-Trp-Lys]-NH₂ 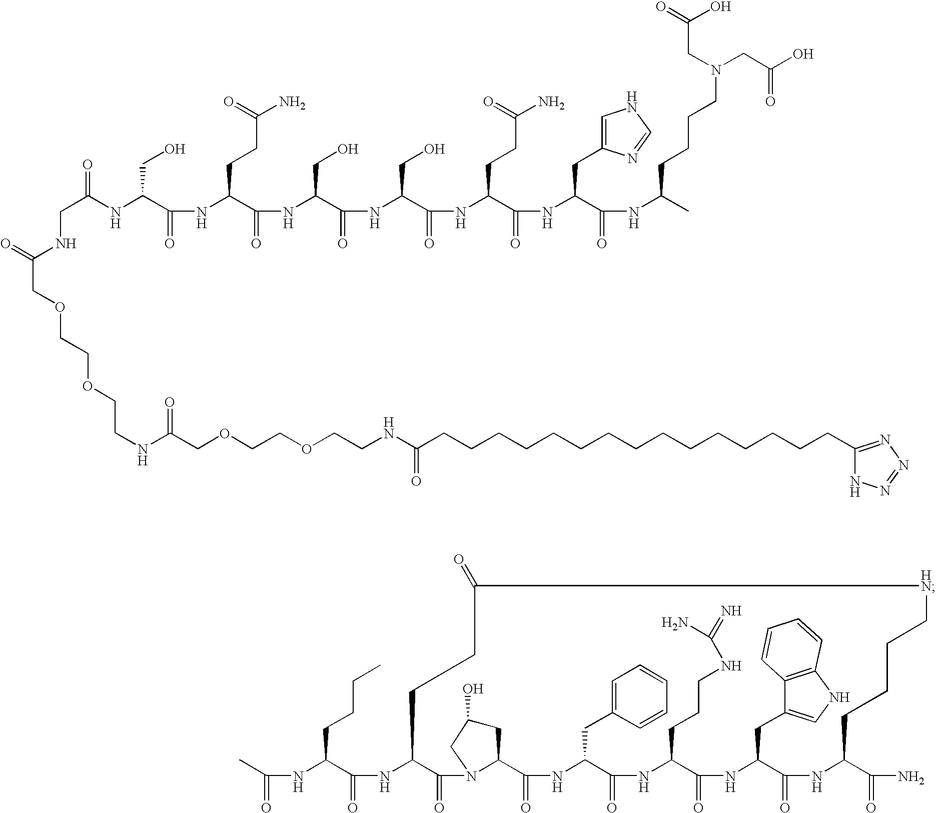
(2-{2-[16-(Tetrazol-5-yl)hexadecanoylamino]ethoxy}ethoxy)acetyl-Gly-Ser-Gln-Ser-Dap(BCMA)-Nle-c[Glu-Hyp-D-Phe-Arg-Trp-Lys]-NH₂ 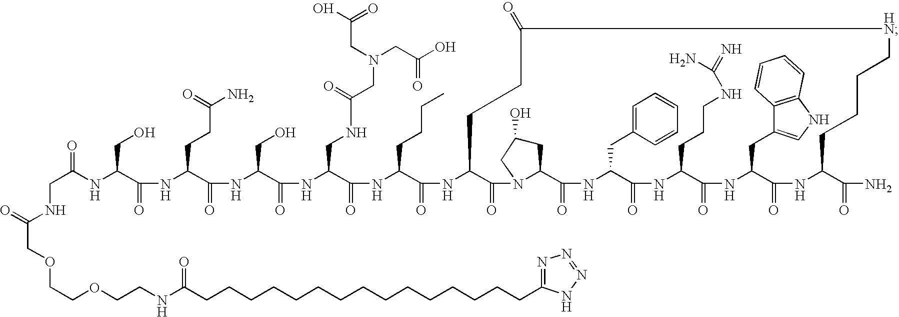
(2-{2-[16-(Tetrazol-5-yl)hexadecanoylamino]ethoxy}ethoxy)acetyl-Gly-Ser-Gln-Ser-Lys(biscarboxymethyl)-Nle-c[Glu-Hyp-D-Phe-Arg-Trp-Lys]-NH₂ 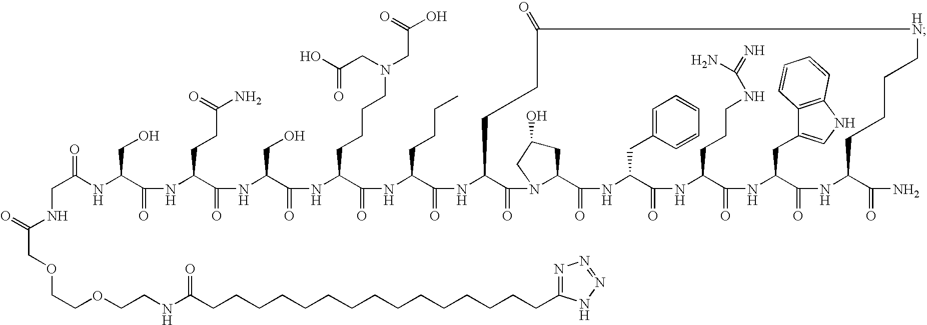
(2-{2-[16-(Tetrazol-5-yl)hexadecanoylamino]ethoxy}ethoxy)acetyl-Gly-Ser-Gln-His-Dap(biscarboxymethyl)-Nle-c[Glu-Hyp-D-Phe-Arg-Trp-Lys]-NH₂ 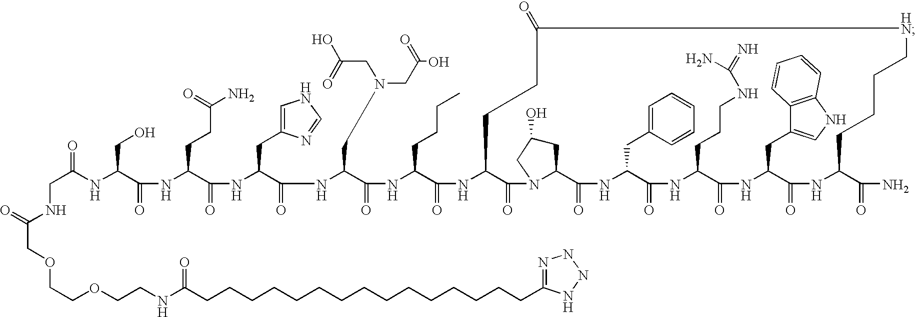
(2-{2-[16-(Tetrazol-5-yl)hexadecanoylamino]ethoxy}ethoxy)acetyl-Gly-Ser-Gln-His-Lys(biscarboxymethyl)-Nle-c[Glu-Hyp-D-Phe-Arg-Trp-Lys]-NH₂ 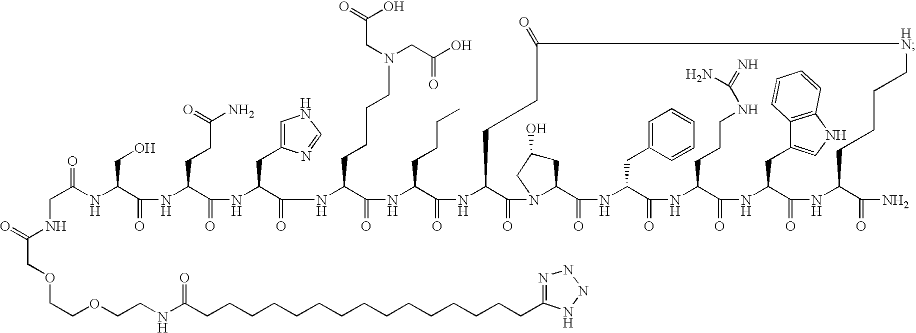
(2-{2-[16-(Tetrazol-5-yl)hexadecanoylamino]ethoxy}ethoxy)acetyl-Gly-Ser-Gln-His-Orn(BCMA)-Nle-c[Glu-Hyp-D-Phe-Arg-Trp-Lys]-NH₂ 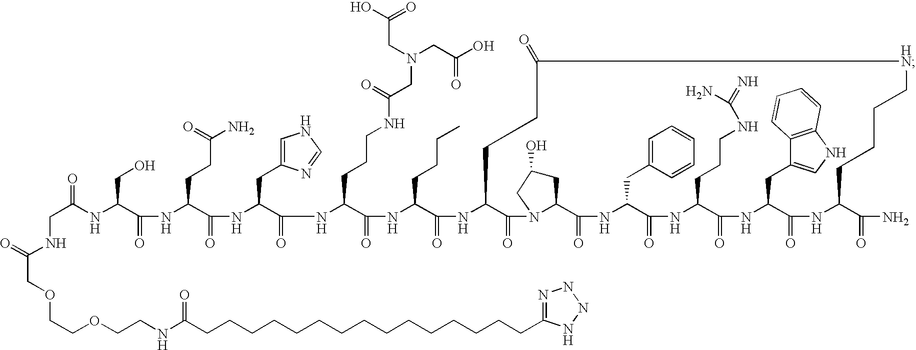
(2-{2-[16-(Tetrazol-5-yl)hexadecanoylamino]ethoxy}ethoxy)acetyl-Gly-Ser-Gln-His-Dap(BCMA)-Nle-c[Glu-Hyp-D-Phe-Arg-Trp-Lys]-NH₂ 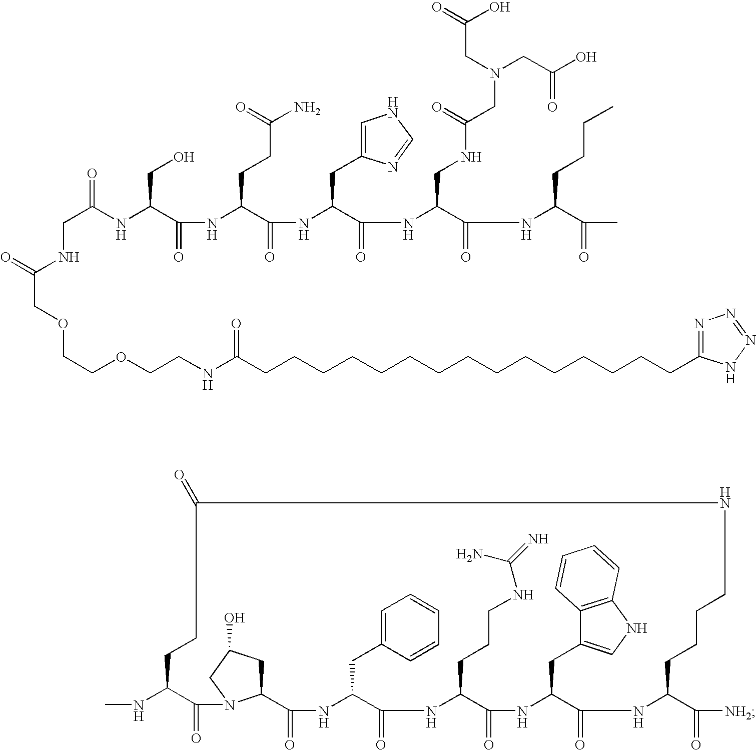
(2-{2-[16-(Tetrazol-5-yl)hexadecanoylamino]ethoxy}ethoxy)acetyl-Gly-Ser-Gln-His-Dab(BCMA)-Nle-c[Glu-Hyp-D-Phe-Arg-Trp-Lys]-NH₂ 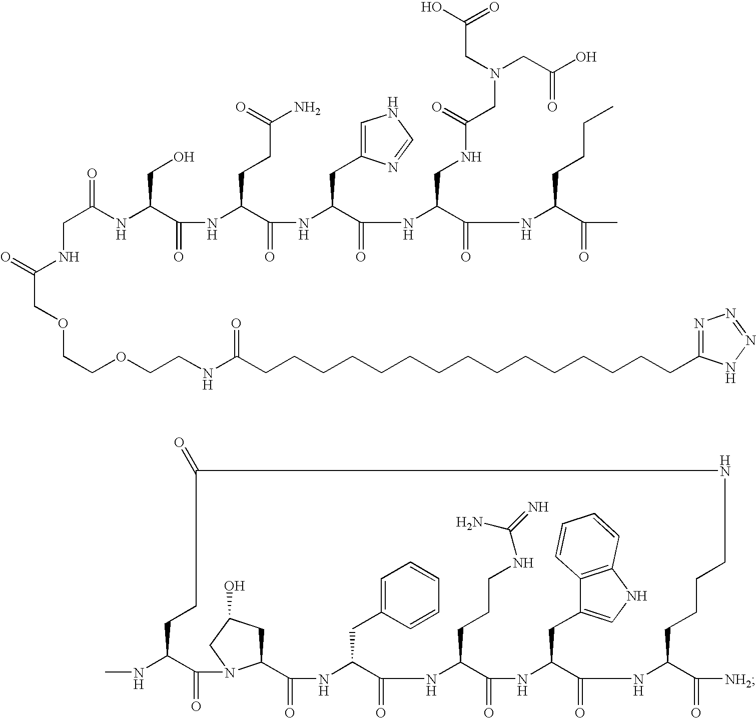
[2-(2-{4-[16-(Tetrazol-5-yl)hexadecanoylsulfamoyl]butanoylamino}ethoxy)ethoxy]acetyl-Gly-Ser-Gln-His-Dap(BCMA)-Nle-c[Glu-Hyp-D-Phe-Arg-Trp-Lys]-NH₂ 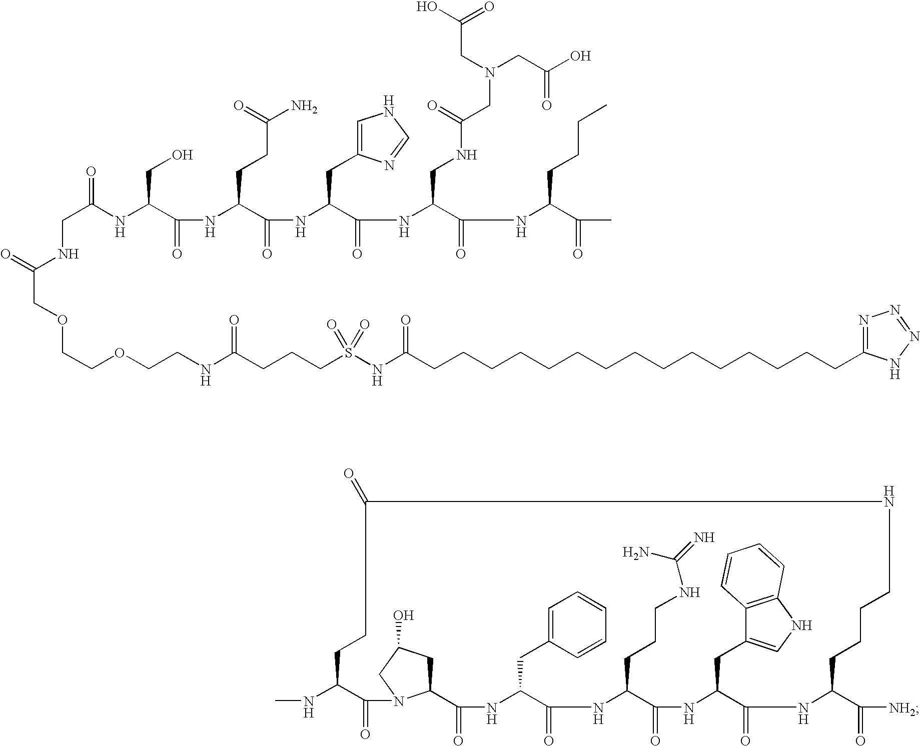
(2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-β-Ala-Lys(biscarboxymethyl)-Nle-c[Glu-H-D-Phe-Arg-Trp-Lys]-NH₂ 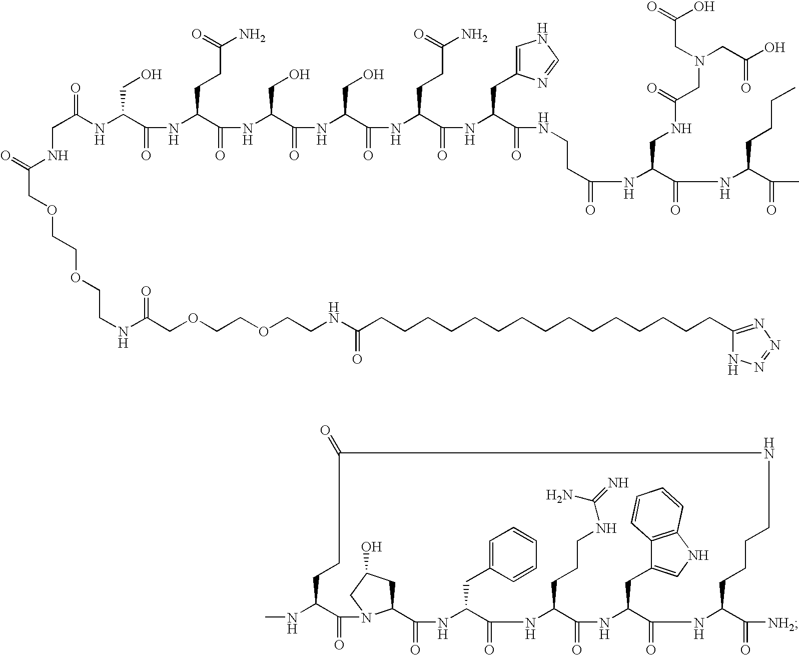
{2-[2-(15-Carboxypentadecanoylamino)ethoxy]ethoxy}acetyl-Gly-Ser-Gln-His-Dap(BCMA)-Nle-c[Glu-Hyp-D-Phe-Arg-Trp-Lys]-NH₂ 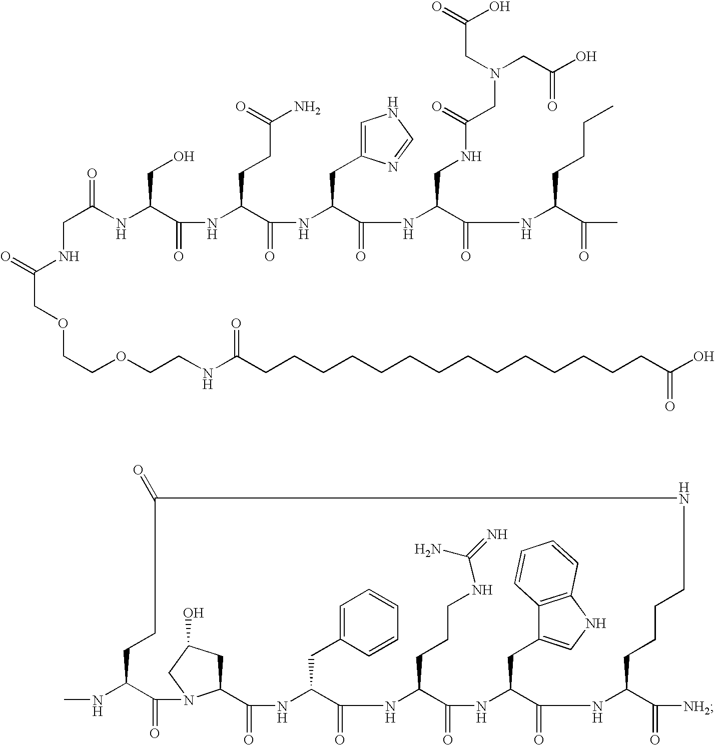
(2-{2-[2-(2-{2-[2-(2-{2-[2-(2-{2-[16-(Tetrazol-5-yl)hexadecanoylamino]ethoxy}ethoxy)acetylamino]ethoxy}ethoxy)acetylamino]ethoxy}ethoxy)acetylamino]ethoxy}ethoxy)acetyl-Gly-Ser-Gln-Ser-Dap(BCMA)-Nle-c[Glu-Hyp-D-Phe-Arg-Trp-Lys]-NH₂ 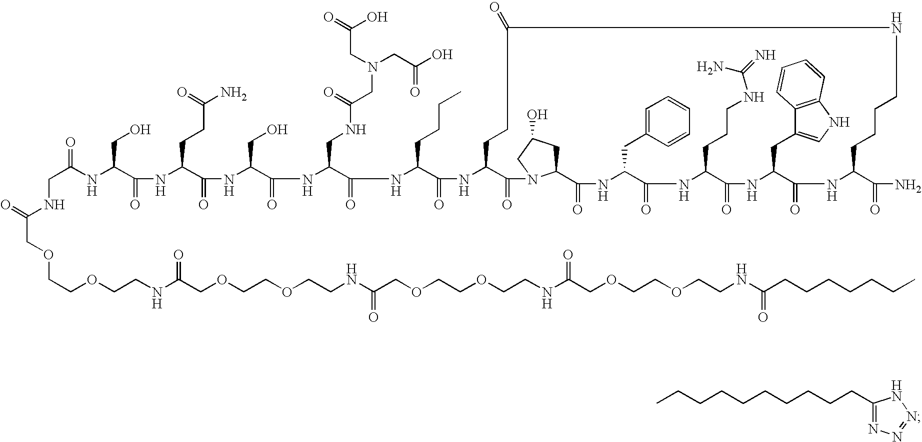
(2-{2-[16-(Tetrazol-5-yl)hexadecanoylamino]ethoxy}ethoxy)acetyl-Gly-Ser-Gln-Ser-Lys(BCMA)-Nle-c [Glu-Hyp-D-Phe-Arg-Trp-Lys]-NH₂ 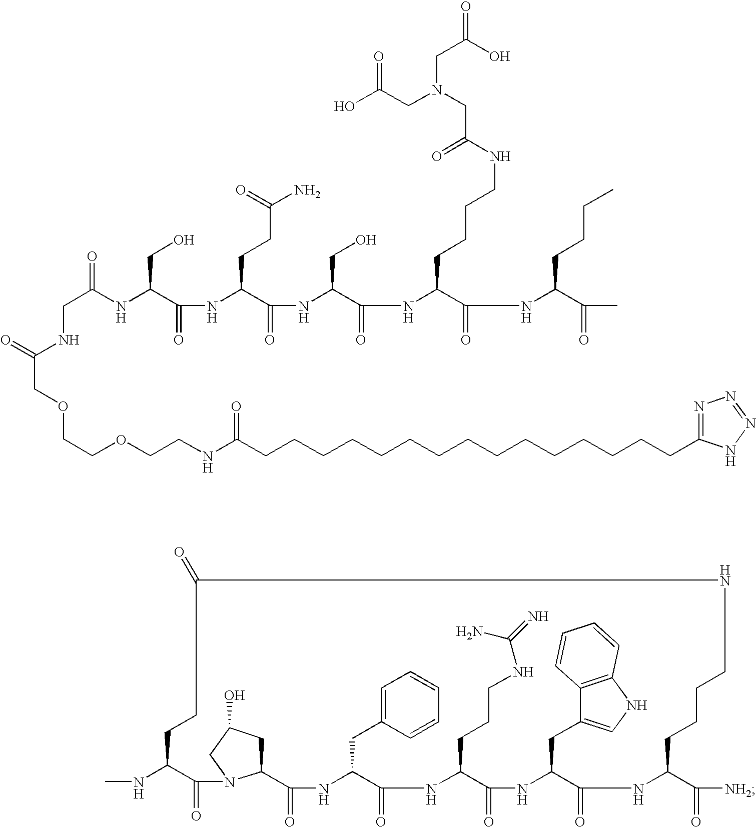
(2-{2-[2-(2-{2-[2-(2-{2-[2-(2-{2-[16-(Tetrazol-5-yl)hexadecanoylamino]ethoxy}ethoxy)acetylamino]ethoxy}ethoxy)acetylamino]ethoxy}ethoxy)acetylamino]ethoxy}ethoxy)acetyl-Gly-Ser-Gln-His-Dap(BCMA)-Nle-c[Glu-Hyp-D-Phe-Arg-Trp-Lys]-NH₂ 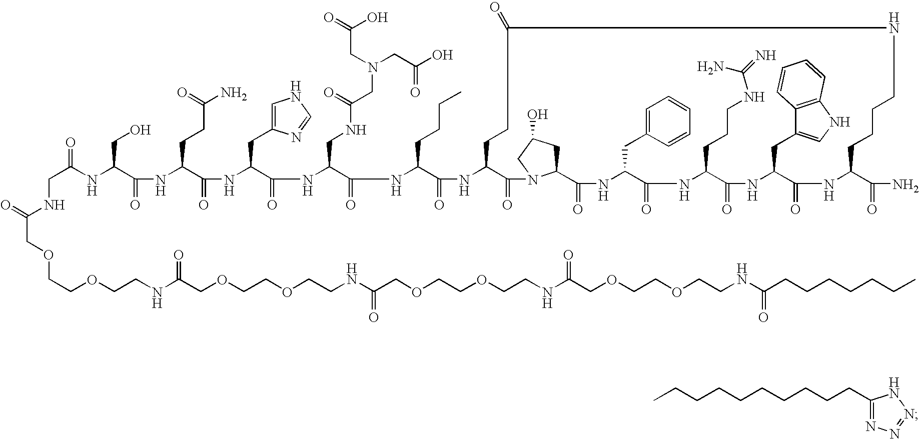
(2-{2-[16-(Tetrazol-5-yl)hexadecanoylamino]ethoxy}ethoxy)acetyl-Glu-Ser-Gln-His-Dap(BCMA)-Nle-c[Glu-Hyp-D-Phe-Arg-Trp-Lys]-NH₂ 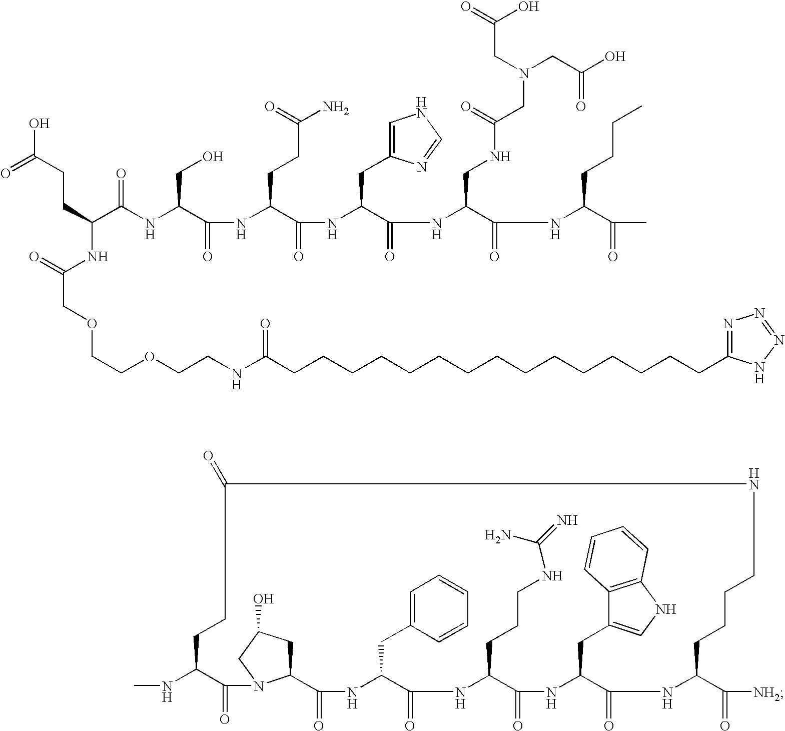
(2-{2-[2-(2-{2-[(S)-4-Carboxy-4-(17-carboxyheptadecanoylamino)butanoylamino]ethoxy}ethoxy)acetylamino]ethoxy}ethoxy)acetyl-Gly-Ser-Gln-His-Dap(BCMA)-Nle-c[Glu-Hyp-D-Phe-Arg-Trp-Lys]-NH₂ 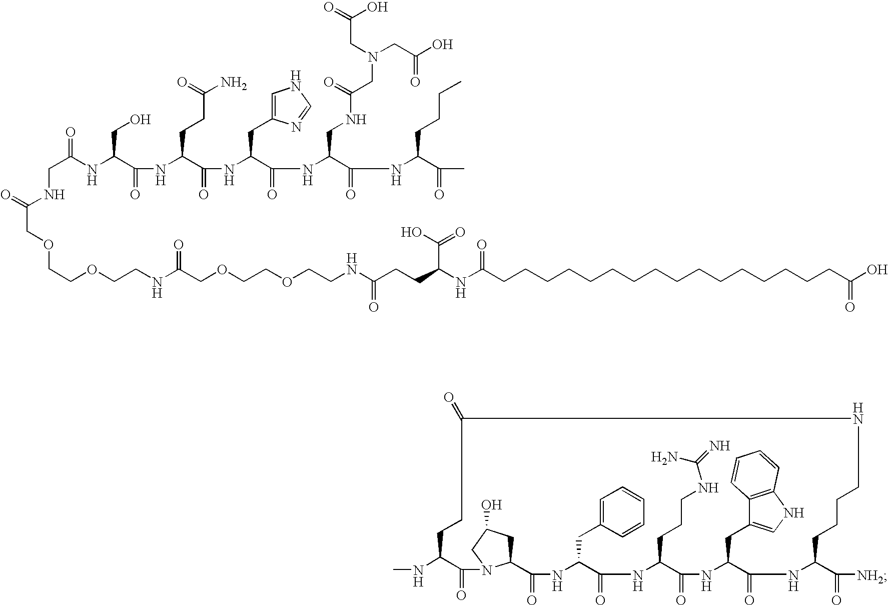
(2-{2-[16-(Tetrazol-5-yl)hexadecanoylamino]ethoxy}ethoxy)acetyl-Gly-Ser-Gln-Tyr-Dap(BCMA)-Nle-c[Glu-Hyp-D-Phe-Arg-Trp-Lys]-NH₂ 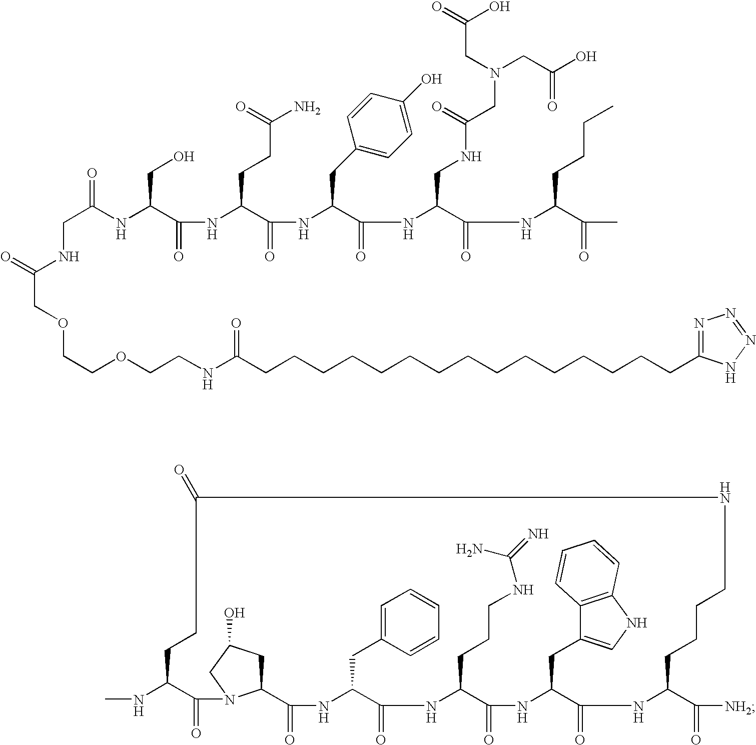
(2-{2-[2-(2-{2-[16-(Tetrazol-5-yl)hexadecanoylamino]ethoxy}ethoxy)acetylamino]ethoxy}ethoxy)acetyl-Gly-D-Ser-Gln-Ser-Ser-Gln-His-β-Ala-Lys(biscarboxymethyl)-Nle-c[Glu-H-D-Phe-Arg-Trp-Lys]-NH₂ 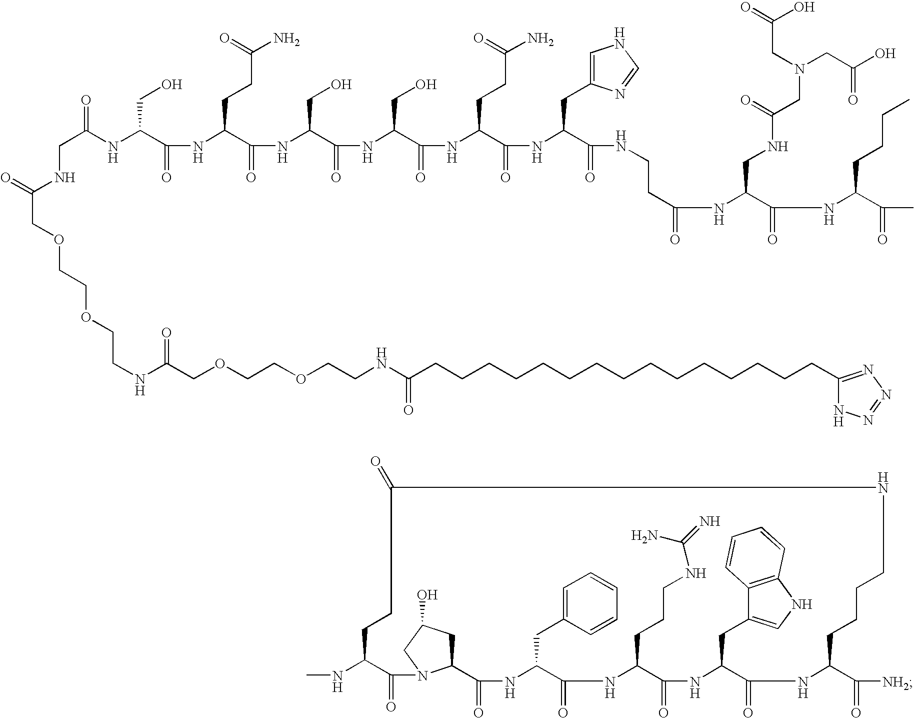
{2-[2-(15-Carboxypentadecanoylamino)ethoxy]ethoxy}acetyl-Gly-Ser-Gln-Ser-Lys(BCMA)-Nle-c[Glu-Hyp-D-Phe-Arg-Trp-Lys]-NH₂ 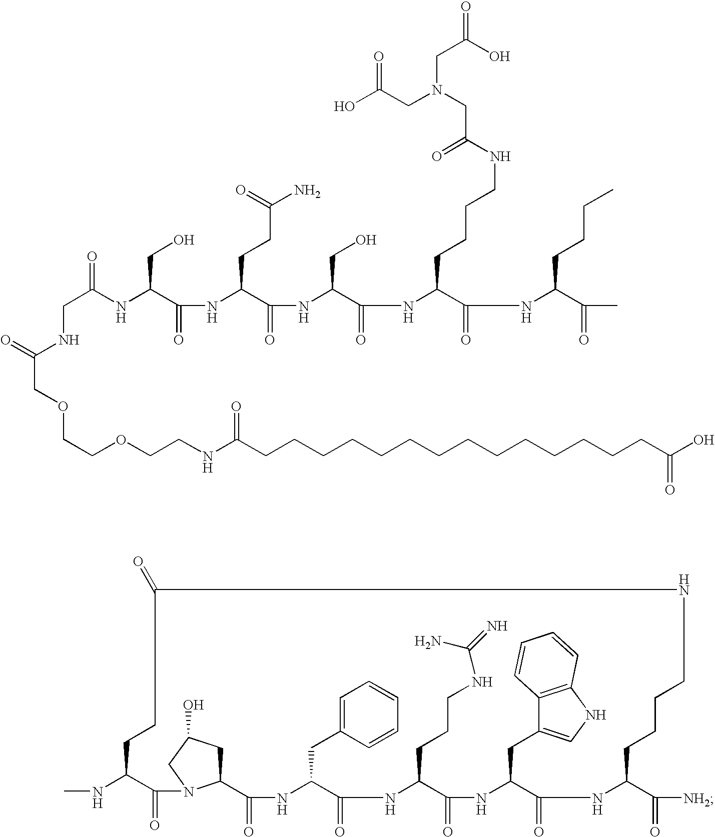
{2-[2-(2-{2-[2-(19-Carboxynonadecanoylamino)ethoxy]ethoxy}acetylamino)ethoxy]ethoxy}acetyl-Gly-D-Ser-Gln-Ser-Ser-Gln-His-Lys(biscarboxymethyl)-β-Ala-Nle-c[Glu-Hyp-D-Phe-Arg-Trp-Lys]-NH₂ 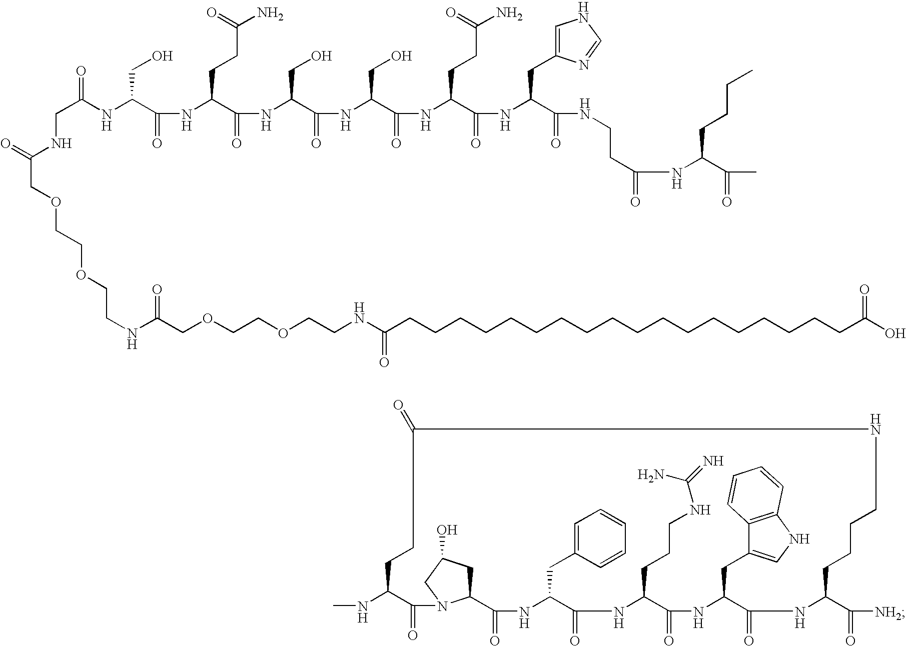
and
(2-{2-[16-(Tetrazol-5-yl)hexadecanoylamino]ethoxy}ethoxy)acetyl-Gly-Ser-Gln-Tyr-Dap(biscarboxymethyl)-Nle-c[Glu-Hyp-D-Phe-Arg-Trp-Lys]-NH₂

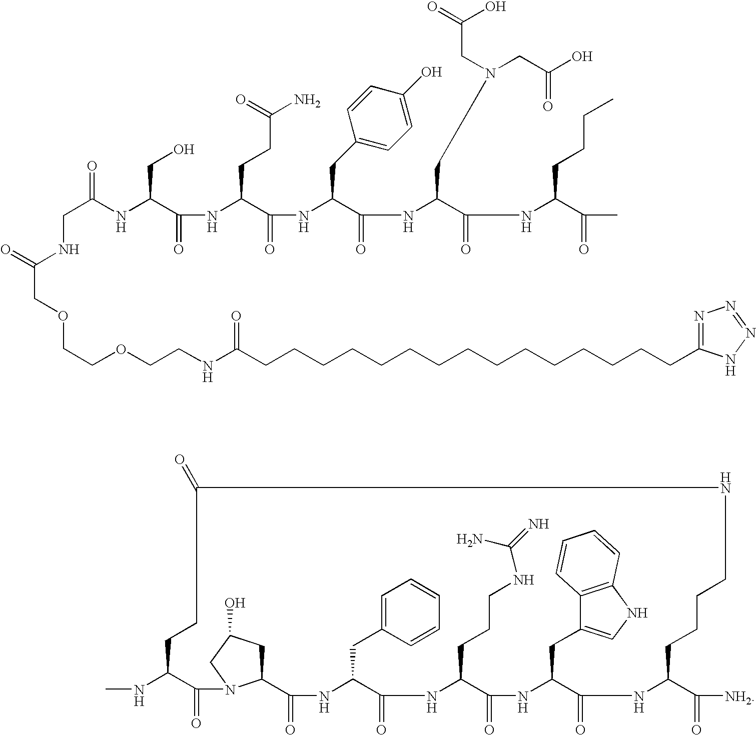

3. (canceled)

4. A method of delaying the progression from non-insulin-requiring type 2 diabetes to insulin-requiring type 2 diabetes, comprising administering to a patient in need thereof an effective amount of a compound according to claim 1, optionally in combination with one or more additional therapeutically active compounds.

5. A method of treating obesity or preventing overweight, comprising administering to a patient in need thereof an effective amount of a compound according to claim 1, optionally in combination with one or more additional therapeutically active compounds.

6. A method of regulating appetite, comprising administering to a patient in need thereof an effective amount of a compound according to claim 1, optionally in combination with one or more additional therapeutically active compounds.

7. A method of inducing satiety, comprising administering to a patient in need thereof an effective amount of a compound according to claim 1, optionally in combination with one or more additional therapeutically active compounds.

8. A method of preventing weight gain after successfully having lost weight, comprising administering to a patient in need thereof an effective amount of a compound according to claim 1, optionally in combination with one or more additional therapeutically active compounds.

9. A method of treating a disease or state related to overweight or obesity, comprising administering to a patient in need thereof an effective amount of a compound according to claim 1, optionally in combination with one or more additional therapeutically active compounds.

10. A method of treating bulimia, comprising administering to a patient in need thereof an effective amount of a compound according to claim 1, optionally in combination with one or more additional therapeutically active compounds.

11. A method of treating a disease or state selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and risk of premature death, comprising administering to a patient in need thereof an effective amount of a compound according to claim 1, optionally in combination with one or more additional therapeutically active compounds.

12. A method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death, comprising administering to an obese patient in need thereof an effective amount of a compound according to claim 1, optionally in combination with one or more additional therapeutically active compounds.

13. (canceled)

14. A pharmaceutical composition comprising a compound according to claim 1 and one or more excipients.

15. (canceled)

16. (canceled)

17. A method according to claim 4, wherein said additional therapeutically active compound is selected from antidiabetic agents, antihyperlipidemic agents, antiobesity agents, antihypertensive agents and agents for the treatment of complications resulting from, or associated with, diabetes.

18. A method according to claim 5, wherein said additional therapeutically active compound is selected from antidiabetic agents, antihyperlipidemic agents, antiobesity agents, antihypertensive agents and agents for the treatment of complications resulting from, or associated with, diabetes.

19. A method according to claim 6, wherein said additional therapeutically active compound is selected from antidiabetic agents, antihyperlipidemic agents, antiobesity agents, antihypertensive agents and agents for the treatment of complications resulting from, or associated with, diabetes.

20. A method according to claim 7, wherein said additional therapeutically active compound is selected from antidiabetic agents, antihyperlipidemic agents, antiobesity agents, antihypertensive agents and agents for the treatment of complications resulting from, or associated with, diabetes.

21. A method according to claim 8, wherein said additional therapeutically active compound is selected from antidiabetic agents, antihyperlipidemic agents, antiobesity agents, antihypertensive agents and agents for the treatment of complications resulting from, or associated with, diabetes.

22. A method according to claim 9, wherein said additional therapeutically active compound is selected from antidiabetic agents, antihyperlipidemic agents, antiobesity agents, antihypertensive agents and agents for the treatment of complications resulting from, or associated with, diabetes.

23. A method according to claim 10, wherein said additional therapeutically active compound is selected from antidiabetic agents, antihyperlipidemic agents, antiobesity agents, antihypertensive agents and agents for the treatment of complications resulting from, or associated with, diabetes.

24. A method according to claim 11, wherein said additional therapeutically active compound is selected from antidiabetic agents, antihyperlipidemic agents, antiobesity agents, antihypertensive agents and agents for the treatment of complications resulting from, or associated with, diabetes.

25. A method according to claim 12, wherein said additional therapeutically active compound is selected from antidiabetic agents, antihyperlipidemic agents, antiobesity agents, antihypertensive agents and agents for the treatment of complications resulting from, or associated with, diabetes.

26. A method of delaying the progression from non-insulin-requiring type 2 diabetes to insulin-requiring type 2 diabetes, comprising administering to a patient in need thereof an effective amount of a compound according to claim 2, optionally in combination with one or more additional therapeutically active compounds.

27. A method of treating obesity or preventing overweight, comprising administering to a patient in need thereof an effective amount of a compound according to claim 2, optionally in combination with one or more additional therapeutically active compounds.

28. A method of regulating appetite, comprising administering to a patient in need thereof an effective amount of a compound according to claim 2, optionally in combination with one or more additional therapeutically active compounds.

29. A method of inducing satiety, comprising administering to a patient in need thereof an effective amount of a compound according to claim 2, optionally in combination with one or more additional therapeutically active compounds.

30. A method of preventing weight gain after successfully having lost weight, comprising administering to a patient in need thereof an effective amount of a compound according to claim 2, optionally in combination with one or more additional therapeutically active compounds.

31. A method of treating a disease or state related to overweight or obesity, comprising administering to a patient in need thereof an effective amount of a compound according to claim 2, optionally in combination with one or more additional therapeutically active compounds.

32. A method of treating bulimia, comprising administering to a patient in need thereof an effective amount of a compound according to claim 2, optionally in combination with one or more additional therapeutically active compounds.

33. A method of treating a disease or state selected from atherosclerosis, hypertension, diabetes, type 2 diabetes, impaired glucose tolerance (IGT), dyslipidemia, coronary heart dis-ease, gallbladder disease, gall stone, osteoarthritis, cancer, sexual dysfunction and risk of premature death, comprising administering to a patient in need thereof an effective amount of a compound according to claim 2, optionally in combination with one or more additional therapeutically active compounds.

34. A method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death, comprising administering to an obese patient in need thereof an effective amount of a compound according to claim 2, optionally in combination with one or more additional therapeutically active compounds.

35. A method according to claim 26, wherein said additional therapeutically active compound is selected from antidiabetic agents, antihyperlipidemic agents, antiobesity agents, antihypertensive agents and agents for the treatment of complications resulting from, or associated with, diabetes.

36. A pharmaceutical composition comprising a compound according to claim 2 and one or more excipients.

37. A method according to claim 27, wherein said additional therapeutically active compound is selected from antidiabetic agents, antihyperlipidemic agents, antiobesity agents, antihypertensive agents and agents for the treatment of complications resulting from, or associated with, diabetes.

38. A method according to claim 28, wherein said additional therapeutically active compound is selected from antidiabetic agents, antihyperlipidemic agents, antiobesity agents, antihypertensive agents and agents for the treatment of complications resulting from, or associated with, diabetes.

39. A method according to claim 29, wherein said additional therapeutically active compound is selected from antidiabetic agents, antihyperlipidemic agents, antiobesity agents, antihypertensive agents and agents for the treatment of complications resulting from, or associated with, diabetes.

40. A method according to claim 30, wherein said additional therapeutically active compound is selected from antidiabetic agents, antihyperlipidemic agents, antiobesity agents, antihypertensive agents and agents for the treatment of complications resulting from, or associated with, diabetes.

41. A method according to claim 31, wherein said additional therapeutically active compound is selected from antidiabetic agents, antihyperlipidemic agents, antiobesity agents, antihypertensive agents and agents for the treatment of complications resulting from, or associated with, diabetes.

42. A method according to claim 32, wherein said additional therapeutically active compound is selected from antidiabetic agents, antihyperlipidemic agents, antiobesity agents, antihypertensive agents and agents for the treatment of complications resulting from, or associated with, diabetes.

43. A method according to claim 33, wherein said additional therapeutically active compound is selected from antidiabetic agents, antihyperlipidemic agents, antiobesity agents, antihypertensive agents and agents for the treatment of complications resulting from, or associated with, diabetes.

44. A method according to claim 34, wherein said additional therapeutically active compound is selected from antidiabetic agents, antihyperlipidemic agents, antiobesity agents, antihypertensive agents and agents for the treatment of complications resulting from, or associated with, diabetes.

45. A method according to claim 30, wherein said additional therapeutically active compound is selected from antidiabetic agents, antihyperlipidemic agents, antiobesity agents, antihypertensive agents and agents for the treatment of complications resulting from, or associated with, diabetes.
